(19) United States
(12) Patent Application Publication
(10) Pub. No.: US 2022/0336058 A1
(43) Pub. Date: Oct. 20, 2022

(54) ANALYSIS METHODS FOR MULTIPLEX TISSUE IMAGING INCLUDING IMAGING MASS CYTOMETRY DATA

(71) Applicants: CEDARS-SINAI MEDICAL CENTER, Los Angeles, CA (US); UNIVERSITY OF SOUTHERN CALIFORNIA, Los Angeles, CA (US)

(72) Inventors: Akil Merchant, Los Angeles, CA (US); Anthony Colombo, Los Angeles, CA (US); Monirath Hav, Los Angeles, CA (US)

(73) Assignees: CEDARS-SINAI MEDICAL CENTER, Los Angeles, CA (US); UNIVERSITY OF SOUTHERN CALIFORNIA, Los Angeles, CA (US)

(21) Appl. No.: 17/642,524

(22) PCT Filed: Sep. 25, 2020
(86) PCT No.: PCT/US20/52879

§ 371 Date: Mar. 11, 2022

Related U.S. Application Data
(60) Provisional application No. 62/905,980, filed on Sep. 25, 2019.

(51) Int. Cl. G16B 40/30; G16B 20/00; G16B 40/10; G01N 33/533

(52) U.S. Cl. CPC G16B 40/30; G16B 20/00; G16B 40/10; G01N 33/533

(57) ABSTRACT

The invention relates to methods for multiplex tissue imaging by methods such as imaging mass cytometry (IMC) and methods for analysis of imaging mass cytometry data. In various embodiments, methods are provided of cell-of-origin analysis and mutational analysis, coupled with spatial parameters derived from tumor clusters in the tumor microenvironment; which reveals signature marker profiles and therapeutic targets for treating cancers including diffuse large B cell lymphoma.

### CROSS-REFERENCE TO RELATED APPLICATIONS

This application includes a claim of priority under 35 U.S.C. § 119(e) to U.S. provisional patent application No. 62/905,980, filed Sep. 25, 2019, the entirety of which is hereby incorporated by reference.

### FIELD OF THE INVENTION

The invention relates to methods for multiplex tissue imaging by methods such as imaging mass cytometry (IMC) and methods for analysis of imaging mass cytometry data.

### BACKGROUND

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Diffuse large B-cell lymphoma (DLBCL) is the most common subtype of non-Hodgkin lymphoma. Although many patients are cured with standard chemo-immunotherapy, up to 40% of DLBCL patients have refractory disease or develop relapse following R-CHOP, or similar regimens, warranting the development of novel, more effective therapeutic strategies for these patients. The composition of the tumor microenvironment (TME) has emerged as an important predictor of DLBCL outcome in gene expression profiling studies. Previous studies have reported that higher proportion of CD4 T cells, dendritic cells, and myofibroblasts predicted better outcome in DLBCL treated with R-CHOP. Other studies reported that increased infiltration of CD8 T cells was associated with better outcome in DLBCL. The role of immune cells such as macrophages and regulatory T cells (T_{REG}) is less clear, with contradictory reports in the literature. Despite recent methodological advances to study the TME in cancer, highly multiplexed immunophenotypic characterization of the tumor immune architecture in DLBCL has not been reported.

The two major molecular sub-types of DLBCL based on cell of origin (COO) are germinal center B-cell (GCB), originating from B-cells in the light-zone, and activated B-cell (ABC), derived from B-cells which have migrated out of the germinal center and committed to differentiation. Generally, GCB sub-type tends to have better overall survival compared to ABC. The COO classifications were originally performed using gene expression and have found widespread clinical application through immunohistochemistry using the HANS algorithm, which identifies GCB and non-GCB subtypes. Other aggressive sub-types include double-expressor, which overexpress MYC and BCL2, and double-hit lymphoma, which have chromosomal rearrangements of MYC and BCL2, or seldomly BCL6. DLBCL have been further subset using somatic copy number alterations and structural variants which sub-stratified COO prognostic signatures. However, integration of COO and mutational analysis with functional and spatial parameters derived from DLBCL TME analyses has not yet been reported.

Programmed cell death receptor 1 ligand (PD-L1) is a member of the B7 family that is expressed on tumor cells and has been reported as a predictor of poor survival in multiple epithelial and hematologic malignancies including DLBCL. Blockade of PD-1/PD-L1 signaling with monoclonal antibodies such as nivolumab and pembrolizumab in patients with relapsed/refractory Hodgkin lymphoma has resulted in high and durable clinical response rates. Unlike in Hodgkin lymphoma, the response rate to PD-1/PD-L1 blockade in relapsed/refractory DLBCL (R/R DLBCL) has been disappointingly low (≤10%), even though PD-L1 is expressed in a significant subset of DLBCL and is associated with poor prognosis.

New technologies allowing for highly multiplexed immune profiling of tissues have not been reported to be applied to the analysis of the tumor microenvironment (TME) in B cell lymphoma. The high cellular density and the co-expression of markers on tumor and immune cells makes the study of these types of tumors particularly challenging. As such, there remains a need for better understanding cancer biology, especially B cell lymphoma biology, to guide the development of new cancer treatments and therapies.

### SUMMARY OF THE INVENTION

The following embodiments and aspects thereof are described and illustrated in conjunction with methods which are meant to be exemplary and illustrative, not limiting in scope.

In various embodiments, the present invention provides a method of performing complex spatial analysis of a tissue sample, comprising: identifying two or more different phenotypic clusters of cells in a tissue sample or an image of the tissue sample, wherein each phenotypic cluster of cells is identified based on two or more closest neighboring cells of a same phenotype to an index cell; determining an average marker intensity of each phenotypic cluster of cells; and comparing the average marker intensity of each phenotypic cluster of cells to an average marker intensity of another phenotypic cluster of cells.

In some embodiments, the method further comprises first using imaging mass cytometry to generate an image of the tissue sample. In some embodiments, the tissue sample is first subjected to mass spectrometry imaging to generate an image of the tissue sample. In some embodiments, the method further comprises calculating a centroid location of each phenotypic cluster of cells by averaging the X,Y coordinates of the two or more closest neighboring cells of the same phenotype to the index cell. In some embodiments, the method further comprises standardizing the distances to the centroids by dividing each centroid distance by the total number of cells of the corresponding phenotype. In some embodiments, the method further comprises scaling the standardized distances by the cohort average abundance of the corresponding phenotype. In some embodiments, the method further comprises mean centering and scaling the standardized distances by their standard deviation to derive Z-scores. In some embodiments, the method further comprises clustering the Z-scores of the distances. In some embodiments, the method further comprises meta-clustering by using the centroid distances per individual case phenotypic clusters. In some embodiments, the method further comprises filtering out distant interactions. In some embodiments, the two or more closest neighboring cells is 5 neighboring cells.

In some embodiments, the methods further comprise identifying a first, tumor-core-immune-desert zone which comprises a phenotypic cluster whose centroid distance to the index cell is no less than a first threshold or the farthest in all clusters, or within which immune activity is lowest in all clusters as characterized by substantially absent of proliferative CD8+ T cells, macrophages or T_{REG} cells; identifying a second, tumor-dispersed-immune-activation zone which comprises a phenotypic cluster whose centroid distance to the index cell is no greater than a second threshold or the shortest in all clusters, or within which immune activity is activated as characterized by substantial presence of proliferative CD8+ T cells, macrophages and T_{REG} cells; and/or identifying a third, tumor-immune-interface zone which comprises a phenotypic cluster whose centroid distance to the index cell is between the first threshold and the second threshold or between the first zone and the second zone, or within which immune activity is suppressed as characterized by substantial presence of exhausted CD8+ T cells and suppressive T_{REG} cells.

In some embodiments, different types of immune cells and/or macrophages and their marker expression profile are measured in one or more of the identified zones.

In some embodiments, the two or more closest neighboring cells is 3, 4, 5, 6, 7, 8, 9 or 10 neighboring cells. In some embodiments, the two or more closest neighboring cells is 5-10, 11-15, 16-20, 21-25, or 26-30 neighboring cells. In some embodiments, the method comprises identifying 3, 4, 5, 6, 7, 8, 9, or 10 different phenotypic clusters of cells. In some embodiments, the method comprises identifying 5-10, 11-15, 16-20, 21-25, 26-30, 31-35, 36-40, 41-45, or 46-50 different phenotypic clusters of cells. In some embodiments, cells are labeled with a label. In some embodiments, the label is selected from the group consisting of antibody label, isotope label, fluorescent label, fluorochrome label, a fluorophore label, and combinations thereof.

In various embodiments, the present invention provides a method for profiling a tumor microenvironment, comprising: performing imaging mass cytometry (IMC) to obtain imaging mass cytometry data of a tumor sample from a subject in need thereof; and performing a method of the present invention to obtain information about the cells in the tumors sample. In some embodiments, the method further comprises using the information to provide a prognosis for survival of the subject. In some embodiments, the method further comprises using the information to determine a treatment for the subject. In some embodiments, the method further comprises administering a treatment to the subject. In some embodiments, the treatment is a combination therapy. In some embodiments, the subject received a treatment for a cancer. In some embodiments, the method further comprises using the information to predict an outcome of a cancer treatment. In some embodiments, the subject has a cancer selected from the group consisting of diffuse large B cell lymphoma (DLBCL), Hodgkin's lymphoma, other non-Hodgkin lymphoma, breast cancer, ovarian cancer, prostate cancer, melanoma, and combinations thereof.

In various embodiments, methods of providing prognosis and/or selecting treatment therapy for a subject with a cancer (e.g., malignant B-cell related disease or cancer) are provided, wherein the marker expression profile in phenotypic clusters of the cancer cells and the different phenotypes of immune cells, macrophages and endothelial cells in various proximity to of the cancer cells are indicative of the subject's responsiveness (or unresponsiveness) to a therapy.

Treatment methods are also provided, which in some embodiments are based on the prognosis outcome from the analysis of the tumor tissue.

In various embodiments, the present invention provides a method for calculating an intensity-weighted abundance score of a marker in a tissue sample, comprising: determining a normalized marker intensity across all cells in the tissue sample or an image of the tissue sample; dividing the tissue sample or the image of the tissue sample into a plurality of blocks based on the quantiles of the total dynamic range of the tissue sample or the image of the tissue sample; assigning an average intensity of cells in each block of the plurality of blocks; counting the number of cells in each block of the plurality of blocks; and determining the intensity-weighted abundance score of the marker by a linear combination of the number of cells in each block and the average intensity of the respective block. In some embodiments, the method further comprises first using imaging mass cytometry to generate an image of the tissue sample. In some embodiments, the tissue sample is first subjected to mass spectrometry imaging to generate an image of the tissue sample. In some embodiments, the tissue sample is first subjected to mass spectrometry imaging to generate an image of the tissue sample. In some embodiments, the plurality of blocks is 10 blocks. In some embodiments, the plurality of blocks is 2-5, 6-10, 10-15, 16-20 blocks. In some embodiments, an intensity-weighted abundance score is calculated for more than one marker in the tissue sample. In some embodiments, an intensity-weighted abundance score is calculated for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 markers in the tissue sample. In some embodiments, an intensity-weighted abundance score is calculated for up to 34 markers in the tissue sample. In some embodiments, an intensity-weighted abundance score is calculated for 36-40, 41-45, 46-50, 51-55, 56-60, 61-65, 66-70, or 71-75 markers in the tissue sample. In some embodiments, a combination of the markers covering T cells, B cells, and Macrophages according to Table 2 is measured in the methods. In some embodiments, cells are labeled with a label. In some embodiments, the label is selected from the group consisting of antibody label, isotope label, fluorescent label, fluorochrome label, a fluorophore label, and combinations thereof.

In various embodiments, the present invention provides a method for determining a patient level summary, comprising: performing a method of the present invention; and taking a weighted sum of the average intensity for each block and the number of cells within each block.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1A-FIG. 1G depict in accordance with various embodiments of the invention that IMC analysis of DLBCL TME identifies marked heterogeneity of immune infiltration and cellular subtypes. FIG. 1A heatmap and tSNE of tumor, CD4, CD8, T_{REG}, macrophages and endothelial meta-clusters, generated by re-phenographing the centroids of each subpopulation. The complete single-cell t-SNE is depicted in FIG. 1H. Statistical testing for marker enrichment denoted with *(p<0.001, ANOVA). FIG. 1B Of the 39.5% of cells comprising the TME, the composition was dominated by CD4, CD8, and macrophages, which make up 92.3% of the immune microenvironment. FIG. 1C Negative correlation between the proportion of CD4 T cells and that of macrophages. FIG. 1D (Left) initial pathologist review revealed various degrees of immune infiltrate in DLBCL. Pseudo-colored images representative of cases with low (top), medium (middle) and high (bottom) degree of immune infiltrate. FIG. 1D (Right) cases ranked in order of absolute proportion of immune cells (9.42% to 90.14%). FIG. 1E analysis of the TME composition showed marked heterogeneity in the distribution of CD4, CD8, T_{REG} and macrophages across cases. FIG. 1F The proportion of CD4 increases with the increasing proportion of immune infiltrate. FIG. 1G The proportion of macrophages decreases with the increasing proportion of immune infiltrate.

FIG. 1H depicts proportions of the meta-clusters in each case, which exhibits overall well distributed cluster, without any case specific meta-cluster. The clustering was performed across each region of interest (ROI), and the centroid (median) expression was used to pool clusters into the meta-cluster level. t-Stochastic neighborhood embedding (tSNE) plotting of the full cohort with major lineage normalized expression maker (CD4, CD8, CD68, FOXP3, CD20, CD31) intensities, which identified major cell components at the cohort level which indicates cluster homogeneity for primary lineage markers at the cohort level.

FIG. 1I depicts the PCA of the phenotype proportions of replicates (top) and the computed Kullback-Leibler (KL) divergence scores (bottom), where the x-axis denotes the case number. Case 26 replicate ROIs had the highest entropy suggesting that in this case the duplicates are heterogeneous. In this study we averaged the replicates at the case level using a mixed-effects linear model.

FIG. 2A-2C depict association between genetic mutations, cell of origin and abundance of sub-cellular phenotypes in DLBCL TME. FIG. 2A shows that sub-phenotypes were created by re-clustering cells using all markers. Heatmap depicts heterogeneity of clinical predictors, genetic mutations and protein expression across all tumor and immune sub-phenotypes. The cluster sizes, strength of association with GCB (χ2 OR>1, p<0.01), non-GCB (χ2 OR<0.9, p<0.01), and the cluster proportion of patient ‘high’ IPI scores greater than 3 portrayed as a cluster percentage. Clusters significantly co-expressing cMYC/BCL2 or cMYC/BCL6 (p<0.01; mixed-effects ANOVA) were determined as ‘double expressor’ based on average normalized cellular intensity. Genetic mutations relevant to DLBCL were included as a cluster proportion (%) depicted on the right. FIG. 2B depicts Replication of significant association between non-GBC COO and genetic mutation statuses. EZH2 (log-odds=−6.08 95% CI: [−6.19, −5.98]), BCL2 (log-odds=−5.57, 95% CI: (−5.67, −5.46)) and SGK1 (log-odds=−4.44, 95% CI: [−4.55, −4.35]) mutations are strongly associated with GCB, whereas MYD88L265P (log-odds=4.19, 95% CI: [4.09, 4.29]), CD79b (log-odds=2.15, 95% CI: [2.13, 2.17]) and NOTCH1/2 mutations are significantly associated with non-GCB. FIG. 2C depicts Correlation between the changes in TME proportion of annotated immune sub-phenotypes and genetic (co)mutation(s). The x-axis depicts the log fold-change of phenotype proportions (%), and the BH q-values are depicted as tagged labels for each point. The y-axis depicts the immune annotation, and the parentheses depict the corresponding sub-cluster. The proportion of memory CD4 phenotype is 20.2% higher in cases with BCL6+CD79b+co-mutations compared to cases without these co-mutations (BH adjusted p=0.116).

FIG. 2D depicts the macrophage CD206 and TIM-3 normalized intensity (y-axis) across macrophage phenotype subsets (x-axis).

FIG. 3A-3D depict spatial arrangement and enrichment of tumor subsets in the context of chemotherapy response status. FIG. 3A shows that the annotated phenotype expression profile is depicted using the mean normalized intensity (z-score). (Left) Box-plot depicts univariate Cox proportional hazard estimates (95% confidence intervals) which identified highly suppressive T_{REG} as significantly hazardous (Cox proportional (log) hazard estimate=0.89, p=0.017) and PD-L1⁺M2-Macrophage as marginally hazardous (p<0.1) at the global TME level. B. FIG. 3B shows that the (left) UMAP projects the structure of each cluster, and adjacent (right) UMAP depicts the enrichment of phenotypes in treatment response. The enriched refractory tumor phenotypes are Ki67-(a), and Ki67+CCR4+ B cell tumor (c), whereas the B cell tumor phenotype enriched in complete responders are denoted as (b). FIG. 3C shows that the global ANOVA model comparing LAG-3 expression (z-score) on each immune subset comparing refractory (REF) subjects to complete responders (CR). The average intensity differences are depicted on the y-axis with 95% confidence interval. The CD45RA⁻ exhausted CD4 LAG-3 average intensity was higher in REF compared to CR (p=0.10), and similarly in highly suppressive T_{REG} (p=9.2e-03). Above the dashed line indicates higher normalized expression in refractory subjects, and below indicates increase in responders. FIG. 3D depicts in order to understand chemo-refractoriness at the hyper-local TME level, spatial interactions of immune phenotypes within 2-3 cell diameters (15 μm) of the tumor cells were computed using 1,000 permutations (p<0.025). The relative proportions (%) of significant spatial interactions of immune phenotypes within the tumor neighborhoods are depicted on the y-axis. Within refractory tumor neighborhoods, PD-L1⁺M2 macrophages had increased co-localization compared to tumor neighborhoods in complete responders and CD4 subsets were co-localized with CR.

FIGS. 3E and 3F depict DLBCL tumor phenotype subsets most enriched across treatment response. FIG. 3E shows tumor and immune phenotypes enriched in refractory subjects. FIG. 3F shows the TME phenotypes related to FIG. 3C, which show that CD45RA⁻ exhausted CD4 and highly suppressive T_{REG} are proportional across treatment response status but have LAG-3 differentially expressed.

FIG. 4A-4D depict that cross-cohort analysis shows differences in proportion and functional states of immune sub-phenotypes between DLBCL and Hodgkin's lymphoma (HL). FIG. 4A depicts the annotated heatmap depicting immune normalized expression of selected sub-phenotypes in both DLBCL and HL TME. Using the relative case proportions per sub-phenotype, the log-odds ratio (left) depicts the strength of association between the sub-phenotypes and DLBCL/HL (p<0.01). Complete heatmap of all sub-phenotypes is shown in FIG. 4G. FIG. 4B depicts after batch normalization, the PCA visually confirms the immune sub-phenotypes identified are well distributed across the two cohorts, indicating no cohort bias. Visual inspection confirmed using k-nearest neighbor batch effect test (kBET), as shown in FIGS. 4E and 4F. FIG. 4C depicts that comparing relative immune proportions between DLBCL and HL shows that CD4 T cells (% difference=−10.2, 95% CI: (−16.2, −4.2)) and T_{REG} (% difference=−8.5, 95% CI: (−11.9, −5.02)) are significantly enriched in HL, while macrophages (% difference=22.1, 95% CI: (14.9, 29.2)) are significantly enriched in DLBCL. CD8 were proportional across disease types (% difference=−3.4 95% CI: (−9.1, 2.4)). FIG. 4D depicts that analyses of cell-state protein expression on each immune subset across the two cohorts show differences in functional states of immune subsets in DLBCL compared to HL. PD-L1 expression on macrophages is significantly higher in HL compared to DLBCL, whereas PD-1 expression is higher on CD8 and T_{REG} in DLBCL compared to HL. TIM-3 expression is significantly higher in DLBCL and is predominantly on CD4 and CD8 T cells.

FIGS. 4E and 4F depict k-nearest neighbor batch correction test (k-BET) scores comparing the TME in DLBCL and Hodgkin's lymphoma. FIG. 4E shows using k-BET, the rejection rates and significance level of the Pearson's Chi-square test are computed. Under the null, the two experiments have no batch effects. The x-axis denotes the TME phenotypes in the 2 experiments, and the y-axis denotes the p-value from the Chi-square test. All the TME phenotypes failed to reject the null hypothesis (CD4 p=0.31, CD8 p=0.49, MAC p=0.43, T_{REG} p-value=0.08). The significance region is depicted in grey. note that we performed the test separately for the 4 phenotypes, but the p-values were not corrected for multiple test corrections. FIG. 4F shows the two-way ANOVA regressing marker expression onto the TME phenotypes and the experiment explanatory categorical variables. We can see that the experiment/disease type feature generally in the upper dots, (which had 2 levels: HL or DLBCL), had very low mean-square errors (MSE). Whereas the TME categorical variable generally in the lower dots (which had 4 levels: CD4, CD8, MAC, T_{REG}) had very high MSE. Hence the expression is more associated with the TME variability and not the disease type.

FIG. 4G depicts ROI protein expression across the TME in DLBCL and Hodgkin's lymphoma. For each ROI, the standardized and batch normalized expression (Z-score) across the TME in DLBCL was compared with that across the TME in Hodgkin lymphoma, in terms of CD4, CD8a, CD68, FOXP3, CD206, and DNA1. By visual inspection, we observed similar dynamic ranges with moderate skewness in CD206 and CD68. The PCA analysis in FIGS. 4A-4D was a patient level average of average markers (TIM-3, CD4, CD68, CD8, FOXP3, PD-1, PD-L1, CD206). This PCA and UMAP at the single cell level visually indicate that the batch correction had minimum cohort/experimental bias using the same markers. FIG. 4G specifically depicts the joint immune phenotype clusters of the DLBCL and HL TME. This was used in supervised annotation of FIG. 4A. Positive “+” calls used 0.49-0.5 cut-offs at the cluster level.

FIG. 5A-5F depict that spatial clustering reveals differences in tumor topology that associate with COO and TME abundance. FIG. 5A shows that neighborhood analysis of cells describes the local arrangement of cells in the tumor. The metric is calculated for each cell by locating the 5 nearest cells belonging to the immune TME, locating the centroid of those nearest neighbors, and measuring the distance from the centroid to the original cell. A smaller distance metric indicates that a cell is embedded within the immune TME, a longer distance denotes exclusion of tumor cells from immune cells. FIG. 5B is a histogram showing the ordered average distance from each tumor topology class to its nearest immune cells. Tumor topology classes were ordered by their distance/proximity to the TME. FIG. 5C shows estimates from a multivariate logistic regression model adjusted for IPI, the log-odds of NGCB vs. GCB for all tumor topology class abundances are depicted. Holding all the other terms constant, tumor_e (log-odds=1.498, p=0.026) is significantly enriched in NGCB, where tumor_c (log-odds=0.46, p=0.08) and tumor_h (log-odds=−0.4, p=0.057) had marginal associations with NGCB and GCB respectively. FIG. 5D shows nine classes of tumor topology were identified and were well distributed across cases, ordered from immune-cold to immune-hot. Intra-tumor spatial heterogeneity is depicted in a representative annotated image from case 26. The first inset shows tumor classes that are more intermixed with the immune cells. The second inset shows tumor spatial arrangement structures similar to the geological topography, with tumor_d situated at the “core”, tumor fat the inner “mantle”, tumor_i at the outer “mantle”, and tumor_c at the “crust” of tumor clusters. FIG. 5E shows that Clark-Evans aggregation index quantifies the level of spatial regularity (index>1), or clustering (index<1), and was applied to the B-cell topology classes in reactive lymph node (RLN) and DLBCL (GCB and NGCB). The Clark-Evans index (box plots) indicated the B-cell topology classes in non-GCB cases were significantly more clustered compared to RLN (Tukey p=0.03), whereas GCB had spatial regularity similar to RLN (Tukey p=0.47). There was marginal difference comparing GCB to NGCB (Tukey p=0.058). This indicates that the spatial distribution of the malignant B cells in GCB tumors more closely resembles the B-cell architecture of normal follicles, while malignant B cell in ABC tumors were more dispersed. FIG. 5F shows that, from the tumor zone reference, the total significant attractions/repulsions of each major cell component were summed in each tumor topology (with 95% confidence interval). Positive values indicate attraction while negative values indicate significant repulsions. The CD4 were enriched within tumor core and mantle neighborhoods, while CD8 were depleted in the core and mantle regions but enriched in the crust and dispersed regions. Macrophages were found in the dispersed, crust and mantle areas but were depleted from the core. The x-axis denotes the major TME, the y-axis denotes the total sum of the significant signed interactions (p<0.05). The tumor zones are ordered by distance to TME from closest (tumor b) to furthest (tumor d).

FIGS. 5G and 5H depict exemplary synthetic cell patterns and spatial sub-classification. Synthetic spatial arrangement showing how tumor topology model was constructed. We generated 4 synthetic patterns with multiple synthetic cell types. FIG. 5G, Image1 and Image2 sub-classified object 1 based on distance to other types. FIG. 5H, Image3 and Image4 identified sub-types for object A based on distance. Image 2 identified the sub-class “1_2” as the “interface” to other object types, whereas class “1_1” was the non-interface class which was determined by their proximity to the other object patterns. Similarly, Image3 identified “A_2” as the interface to other point patterns. Lastly, Image4 identified an ordered distance with the furthest pattern within “A” class to “B” class was “A_1”, and the closest was “A_6”. Image4 demonstrates that the distance classification can order a pattern by distance to the other patterns.

FIG. 5I depicts B-cell topology sub-classification. Using 5-NN centroid to sub-classify B-cell major phenotype in the lymph node identified 7 B-cell topography classes based on proximity to other immune cells. Importantly, by clustering in terms of distance to the nearest immune cells (unsupervised), we classified B-cells which identified heterogeneity in the light and dark zones. The image rendering of the lymph node showing only the major cell phenotypes annotated. The raw ablated images for CD20, Ki67, and PD-1. B-cell sub-classification using the 5-NN centroid was able to identify the heterogeneity of the B-cells in the light (Cluster 3) and dark (Cluster 4) follicular zones. We classified cells based on nearest immune cell.

FIG. 5J depicts the spatial interaction using 1,000 permutations (d=15 microns). We identified that T-follicular helper cells spatially co-localized significantly with B-cell topography classes (4, 3 and 5) from FIG. 5I.

FIG. 5K-5M depict tumor topography classes analysis details. FIG. 5K shows that the logistic regression utilized above was compared against a random null model which permuted the topography labels 250 times, and re-performed the logistic regression stratified on the tumor topological cluster labels. The AIC scores were compared between the null model and the observed AIC model to determine robustness. In FIG. 5L, each tumor phenotypic sub-cluster HLA-DR expression (Z-score) is depicted on the x-axis. The spatial interactions (1,000 permutations, interaction distance=15 microns) from the CD4 phenotypes to tumor phenotypic sub-clusters was computed (y-axis). We did not see a linear correspondence between the tumor sub-clusters HLA-DR expression and spatial interactions with CD4. This is expected because we do not incorporate immune interactions into clustering. Instead of the tumor phenotypic sub-clusters, we measured the spatial interactions from CD4 to the tumor spatial zones. This analysis indicated that by ordering the tumors by distance to the TME, we see a significant linear correspondence between HLA-DR expressed on the tumor and the number of CD4 interactions. In FIG. 5M, each ROI used 1,000 permutations to determine the spatial interaction (interaction distance=15 microns) from the TME to tumor spatial topology classes. The (y-axis) depicts the total cases with significant interactions (p=0.025) divided by total cases averaged across the tumor topology classes (x-axis). The x-axis depicts the TME based on interactions with dispersed tumors (a, b, e, g, h), semi-penetrating TME by interactions with periphery tumors (c, f, I), and penetrating by interactions with tumor core (d).

FIG. 6A-6D depict DLBCL Comprehensive spatial interactions between TME and tumor topology, functional state profile, mutation, and patient clinical association. FIG. 6A shows a heatmap of spatial interactions between immune sub-phenotypes and tumor topology classes using 2-3 cell diameters (15 μm) radius for interaction distance, and significance determined from 1,000 permutations of phenotype labels (p=0.025). Also illustrated here are their multivariate association (IPI adjusted) with genetic mutations and Hans COO. Tumor_c, which was significantly associated with CD79b mutation and TP53/CD79b co-mutation, shows interactions with highly suppressive T_{REG} and TIM-3⁺PD-1⁺LAG-3⁺ exhausted CD8 T cells. Tumor_e, which is enriched in NGCB subtype, interacts with activated and exhausted CD8 T cells, and exhausted CD4 T cells. In FIG. 6B, the interaction between the tumor core and CD4 sub-phenotypes with increasing expression of CXCR3 shows positive linear association. The x-axis is the mean normalized intensity of CXCR3, and the y-axis is the number of ROI with statistically significant interactions between the tumor core and CD4 phenotypes (1,000 permutations, p=0.025). In FIG. 6C, the summary description of the DLBCL tumor topology, which depicts the order from dispersed tumor domains (left) to the tumor core regions (right), and its associations with Hans cell-of-origin, CD79b mutations, and TME phenotypes which are embedded within each tumor neighborhood topology. Each zone is associated with an immune status: immune active, immune suppressed, and immune desert based on the phenotypes and abundance of immune cells in each zone. Ordering of tumor spatial clusters allows for the association of CXCR3 expression with tumor penetrating immune phenotypes. In FIG. 6D, the nine tumor spatial clusters were grouped into three zones: dispersed, mantle/crust, and core and the TME phenotypes surrounding (15 μm) the tumor cells in each zone were examined. Significant interactions (p=0.025) are displayed with positive values indicating enrichment and negative values depletion. Immune cell around tumors in the dispersed area were enriched for highly proliferative and activated phenotypes. Cells associated with immune suppression such as exhausted CD8 T cells and highly suppressive T_{REG} were enriched around tumor cells in the mantle/crust area. Most immune phenotypes, except the highly suppressive T_{REG} were depleted around tumor cells in the core region. The x-axis depicts the tumor topology, and the y-axis indicates the significant interaction of the TME as a proportion (%).

FIG. 6E depicts Ki67 expression spatial heterogeneity on B-cell topology. A global mixed effects linear model across all labeled sub-clusters on Ki67 expression was performed. The depicted plot here shows the estimates of the tumor topology estimated from the global model which identified that the tumor core was moderately proliferative compared to the global Ki67 expression (estimate=0.43, p<0.001), the mantle (estimate=0.38, p<0.001), transition zone (estimate=0.41, p<0.001), crust (estimate=0.39, p<0.001), and dispersed tumors (estimate=0.26, p<0.001). we observed a decreasing intensity of Ki67 as the tumor zones in the context of TME distance classification. The tumor topologies were mildly proliferative overall, and the tumor topologies were mostly different compared to the dispersed tumor domain.

FIG. 7A-FIG. 7H depict in accordance with various embodiments of the invention, survival analysis using M-score shows complex tumor phenotype co-expressing PD-L1/TIM-3/CCR4 to be an independent predictor of poor OS in DLBCL. See also FIG. 13A-FIG. 13E and FIG. 14A-FIG. 14G. FIG. 7A the first two representative histograms show bimodal distribution of normalized intensity of phenotypic markers CD3 and CD8, which allows separation between positive and negative calls at 0.5 cut point. The last two histograms depict a challenge in determining an optimal cut point for inducible markers such as PD-1 and CXCR3 as their intensity distribution either falls below or above the chosen 0.5 cut point. FIG. 7B the bar graphs show reasonable distribution of the positive calls at 0.5 cut point for phenotypic markers CD3, CD8, FoxP3 and CD68 on CD4, CD8, T_{REG} and macrophage populations previously identified by meta-clustering. FIG. 7C PD-L1 intensity was tuned using IHC and RNAscope data. At a cut point of 0.34, the linear model which regressed the PD-L1+ relative abundance onto IHC and RNAscope scores has the highest average F-values. FIG. 7D the bar plot shows distribution of complex immune phenotypes in DLBC1'S TME. PD-L1/TIM-3/CCR4 triple positive CD8 and CD4 represent the two most dominant T cell subsets in the TME and account for 17.88% and 14.01% of all CD8 and CD4 T cells, respectively. PD-1+ T cells are rare. FIG. 7E donut plot shows that 77.38% of the PD-L1/TIM-3/CCR4 triple positive tumor cells express BCL2. This complex tumor phenotype makes up 9.96% of all tumor cells and is significantly associated with refractory disease (chi-squared p<0.0001). FIG. 7F and FIG. 7G survival analyses using the optimal cut points and M-score show the same level of significance (p=0.003) for association between OS and tumor co-expressing PD-L1/TIM-3/CCR4, yet slightly improved C-index was observed in the model using M-score (C=0.85) as compared to the model using optimal cut points (C=0.80).

FIG. 711 higher M-score for complex tumor phenotyping co-expressing PD-L1/TIM-3/CCR4 is an independent predictor of poor OS in multivariate model after adjusting for IPI score (P=0.003, C=0.83).

FIG. 8A-FIG. 8D depict in accordance with various embodiments of the invention, incorporation of CD8 spatial neighborhood feature into phenotypic profiling improved survival prediction in DLBCL. FIG. 8A and FIG. 8C univariate survival analysis show a trend for association between poor OS and higher M-score for PD-L1-expressing tumor (FIG. 8A) and endothelium (FIG. 8C). FIG. 8B and FIG. 8D the survival prediction for the above phenotypes improves when controlling for their spatial proximity to CD8 T cells.

FIG. 9A-FIG. 9G depict in accordance with various embodiments of the invention, spatial analysis reveals distinct types of CD8 neighborhoods associated with clinical outcome in DLBCL. FIG. 9A schematic drawing showing neighborhood-based spatial model for CD8 T cell interaction. The model is based on average distances from CD8 to the centroids of 5 nearest neighboring CD4, T_{REG}, macrophages, endothelial, and tumor cells. FIG. 9B UMAP (left) and heatmap (right) showing 11 spatial meta-clusters and 1 unclassified CD8 cluster identified by unsupervised clustering of the average distances between CD8 T cells and the centroids of their 5 nearest neighboring CD4, T_{REG}, macrophages, endothelial, and tumor cells. FIG. 9C box plot showing the average distances (um) from CD8 to the centroids of the 5 neighboring CD4, T_{REG}, tumor, macrophages and endothelial cells. Each CD8 spatial interaction pattern is distinctive, reflected by different average distances from CD8 to the centroids of each phenotype. FIG. 9D bar graph showing heterogeneity of CD8 spatial cluster/neighborhood distribution across cases (top). Visual inspection of images from case 27 (bottom left) validates the CD8 spatial interaction patterns in clusters 10, 6 and 1 (insets, bottom right). FIG. 9E dot plot showing the odds ratio of finding a particular spatial cluster in refractory cases over cases with complete remission. These spatial neighborhoods are clinically relevant. The unclassified CD8 clusters and clusters 1, 2, 4 and 7 (“hazardous” spatial neighborhoods) had at least 3 times higher odds of being identified in refractory cases compared to clusters 6, 9, 10 and 11 (“protective” spatial neighborhoods). FIG. 9F shows a table illustrating the different patterns of CD8 T cell interaction in the hazardous versus protective spatial neighborhoods. In the hazardous neighborhoods, CD8 T cells generally interact with macrophages, whereas in the protective neighborhoods, CD8 T cells tend to interact with CD4. FIG. 9G representative snapshots from cases 33 and 26 showing interaction between CD8 and CD4 T cells in the protective neighborhood (left) and between CD8 and macrophages in the hazardous neighborhood (right).

FIG. 10A and FIG. 10B depict in accordance with various embodiments of the invention, CD8 functional heterogeneity is determined by their spatial interaction with other immune cells. FIG. 10A when ordered by hazards ratio for REF/CR, Ki-67 and granzyme-B expressions on CD8 tend to covary, demonstrating a coherent pattern of initial activation followed by suppression. Ki-67 and Granzyme-B average expressions on CD8 in “protective” cluster C11 are higher than their average expressions on CD8 in “hazardous” cluster C2. FIG. 10B the trend diagrams show that PD-1 seems to decrease from protective to hazardous neighborhoods, while TIM-3 expression shows the opposite pattern.

FIG. 11A-FIG. 11C depict in accordance with various embodiments of the invention, analysis of inducible marker expression on CD8 neighboring phenotypes yields insights into the functional statuses of immune cells in the TME. FIG. 11A schematic of analyses of average protein expression on CD8's 5 nearest neighbors (5NN) in the observed model (upper left) versus average protein expression on any random 5 cells in the null model (upper right). To calculate Z-score, the observed average protein intensity on the 5NN of a particular neighboring phenotype is compared to the average protein intensity on any random 5 cells of that same phenotype. High Z-score means the average protein intensity in the observed model is higher than that in the random/null model, and vice versa (bottom graphs). FIG. 11B heatmap showing average expressions of key inducible markers Ki-67, Granzyme-B, PD-L1, TIM-3, FoxP3, CXCR4, CD206, CCR4, CXCR3, and Tbet on CD8 neighboring phenotypes across all spatial meta-clusters. FIG. 11C schema illustrating immune suppressive micro-region in “hazardous” cluster C2 and immune activating micro-region in “protective” custer C11. In C2, dysfunctional CD8 with low Ki-67 and Granzyme-B, M2-like macrophages with high CD206, and highly suppressive TREG with high CCR4 experssion are observed. In C11, the micro-region is composed of activated CD8 cells, Th1-like CD4 cells, and less suppressive TREG phenotypes, characterized by high Ki-67 and granzyme-B on CD8, high CXCR3*Tbet joint expression on CD4, and low CCR4 on TREG. The average joint expression of PD-L1/TIM-3/CCR4 on tumor cells is higher in C2 compared to C11.

FIG. 12A and FIG. 12B depict in accordance with various embodiments of the invention, analysis of inducible marker expression in CD8 spatial neighborhoods yields insights into the functional statuses of immune cells in the TME. FIG. 12A box plots showing the variation in average (joint) intensity of key inducible markers Ki-67, Granzyme-B, PD-L1, TIM-3, ICOS, FoxP3, and CXCR3*Tbet on different cell populations across all spatial meta-clusters. FIG. 12B schema illustrating the different functional statuses of immune cells in “protective” versus “hazardous” spatial neighborhoods. In the “protective” spatial neighborhoods, we observed the presence of activated CD8, Th1-like CD4, and less suppressive T_{REG} phenotypes, characterized by high Ki-67 and granzyme-B on CD8, high CXCR3*Tbet joint expression on CD4, and low FoxP3, TIM-3, PD-L1 and ICOS on T_{REG}. The opposite was observed in the “hazardous” spatial neighborhoods. TIM-3 is upregulated both on T cells and tumor cells in the “hazardous neighborhood”.

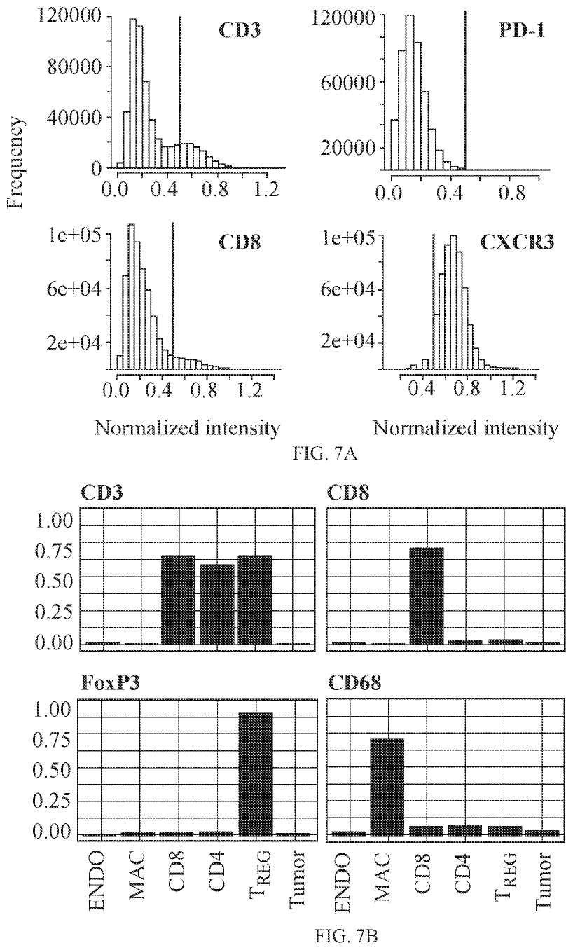

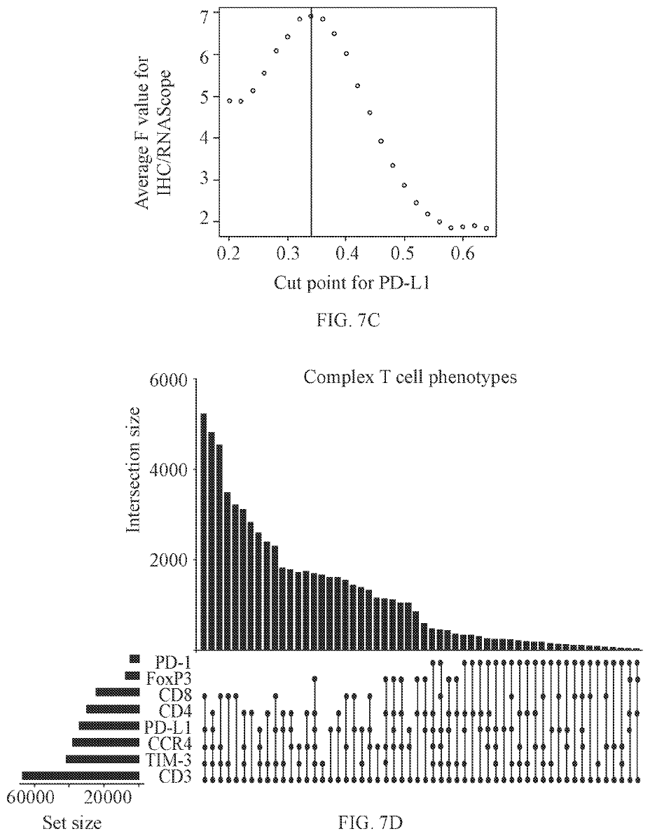

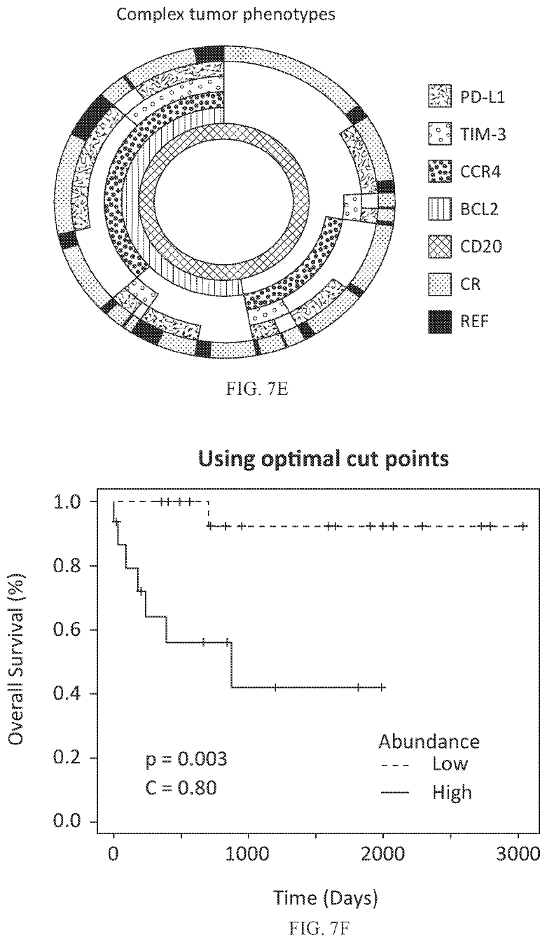

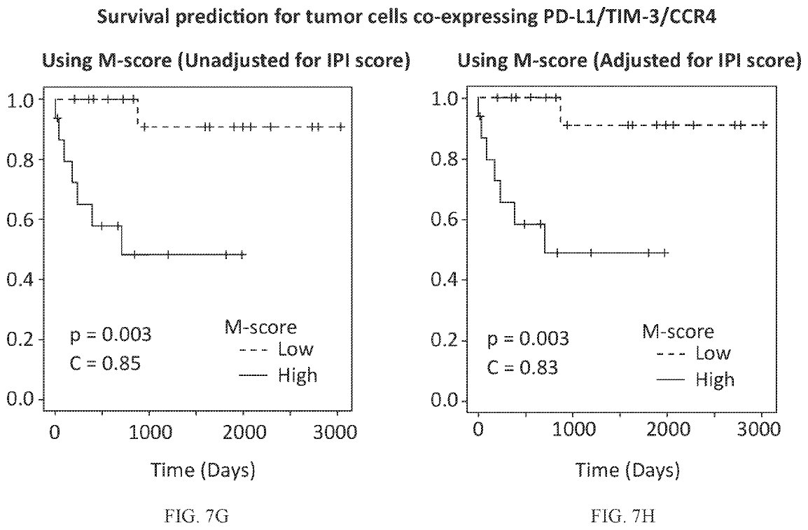

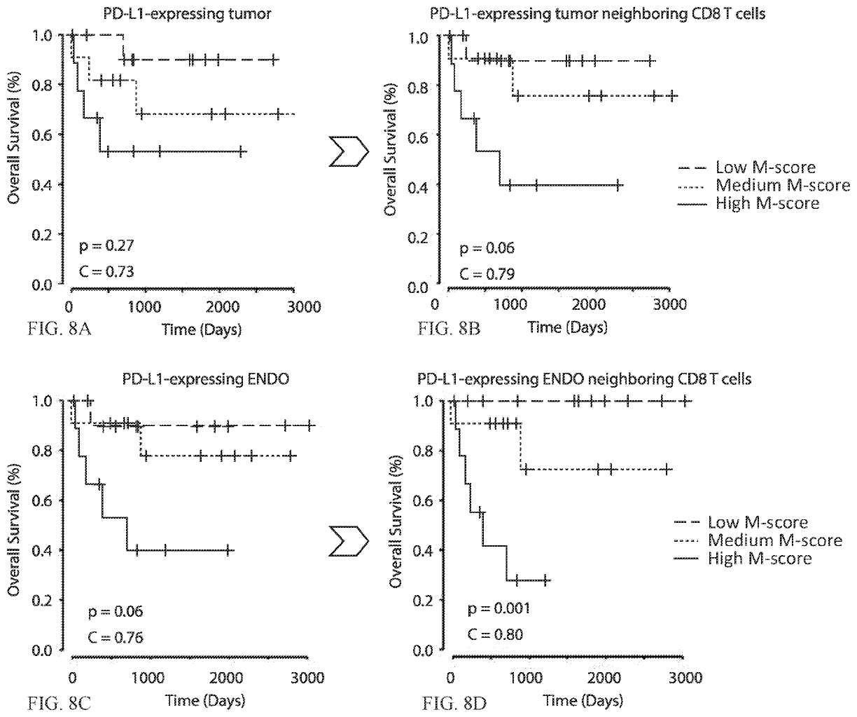

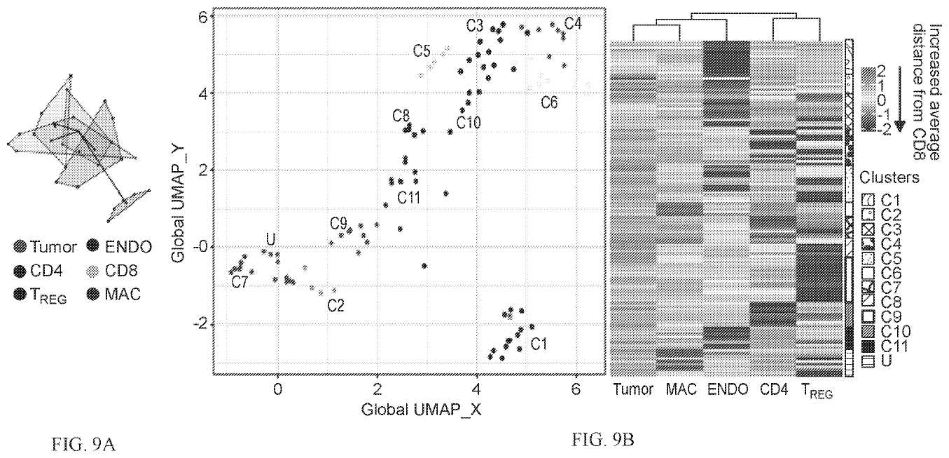

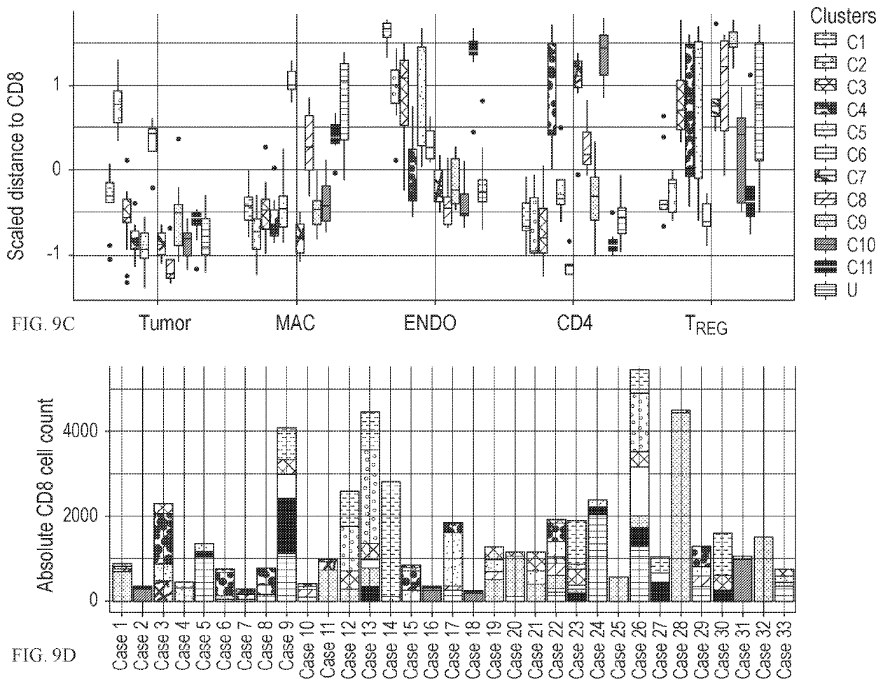

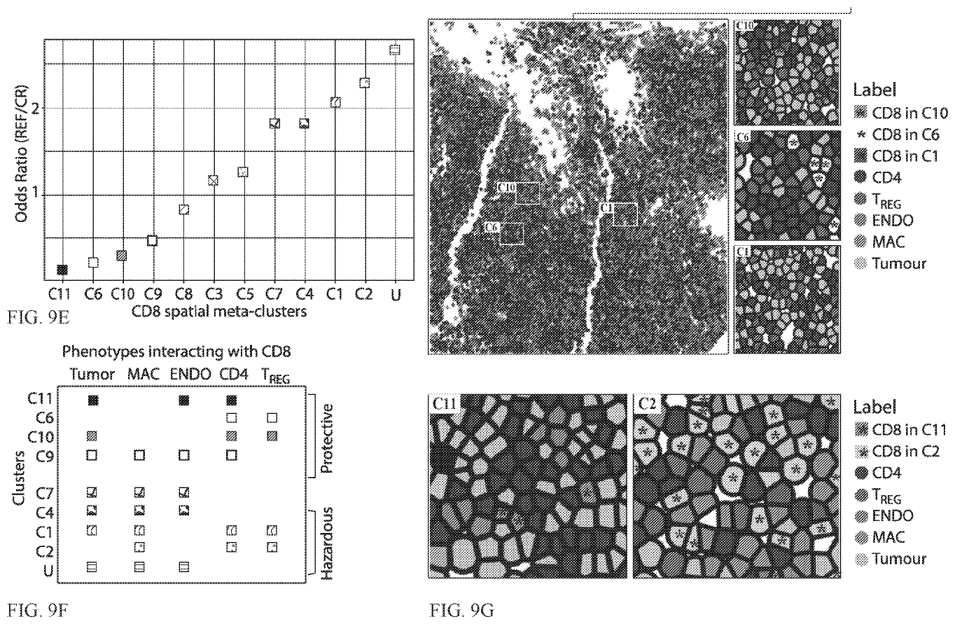

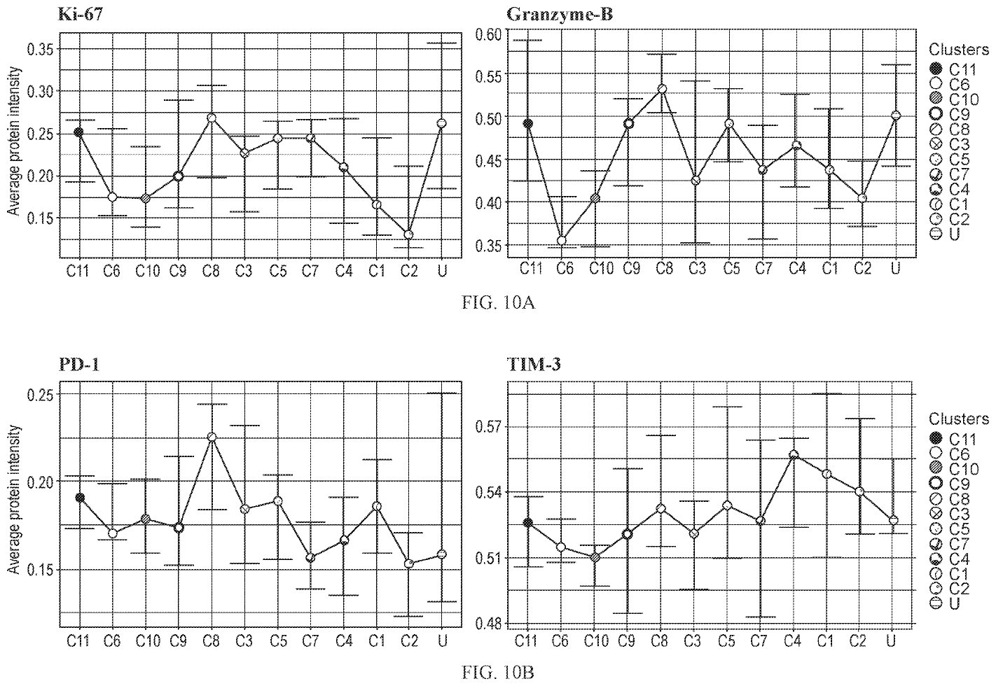

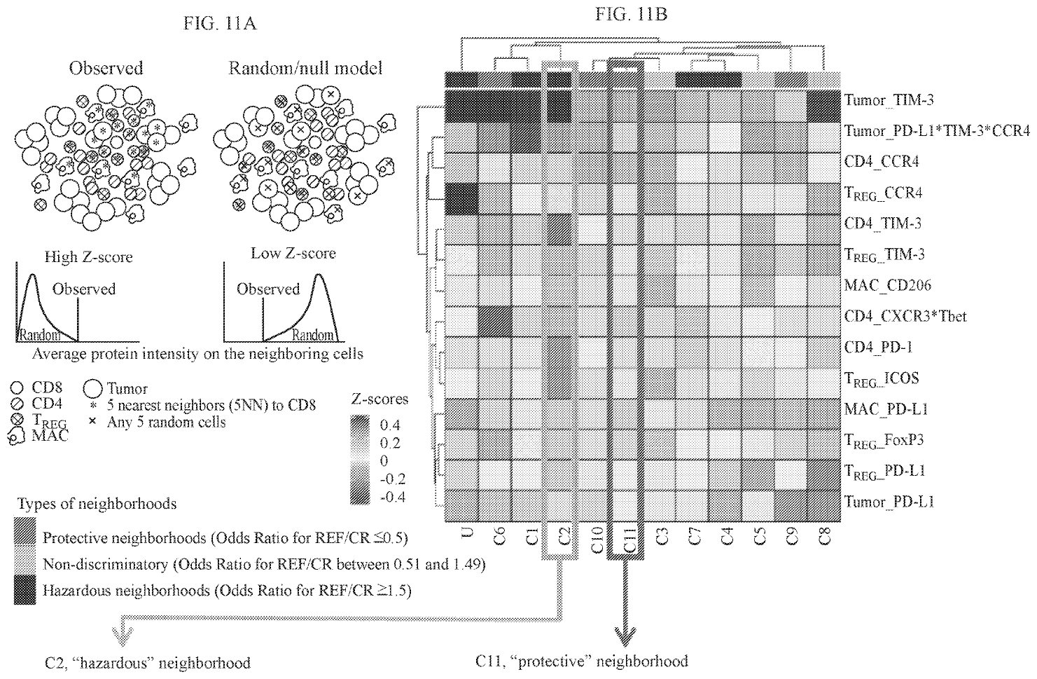

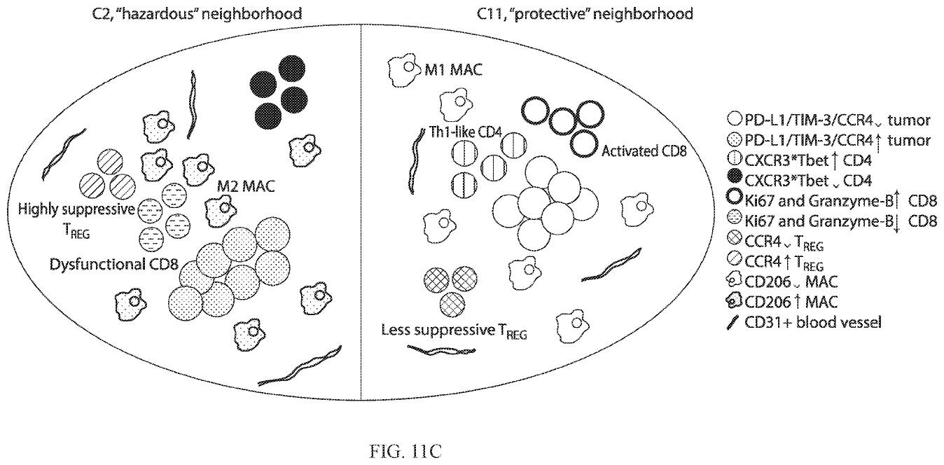

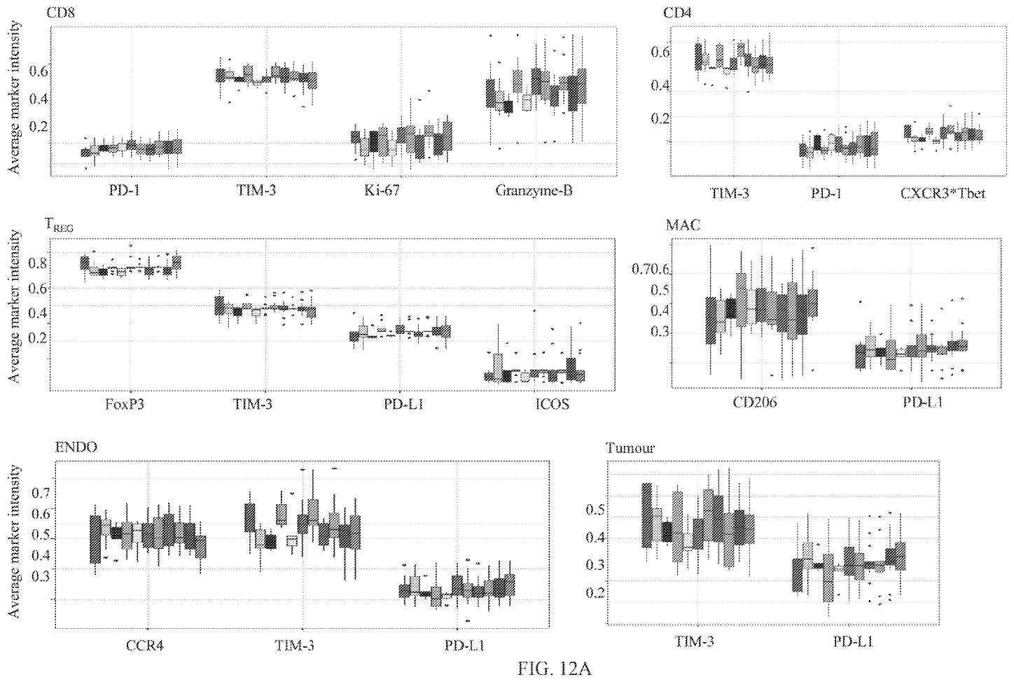

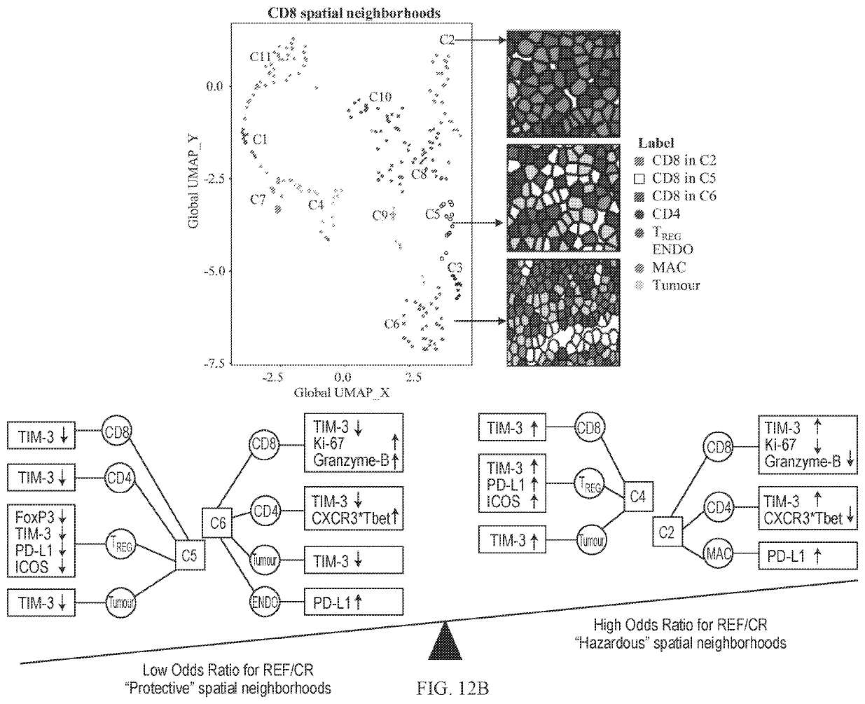

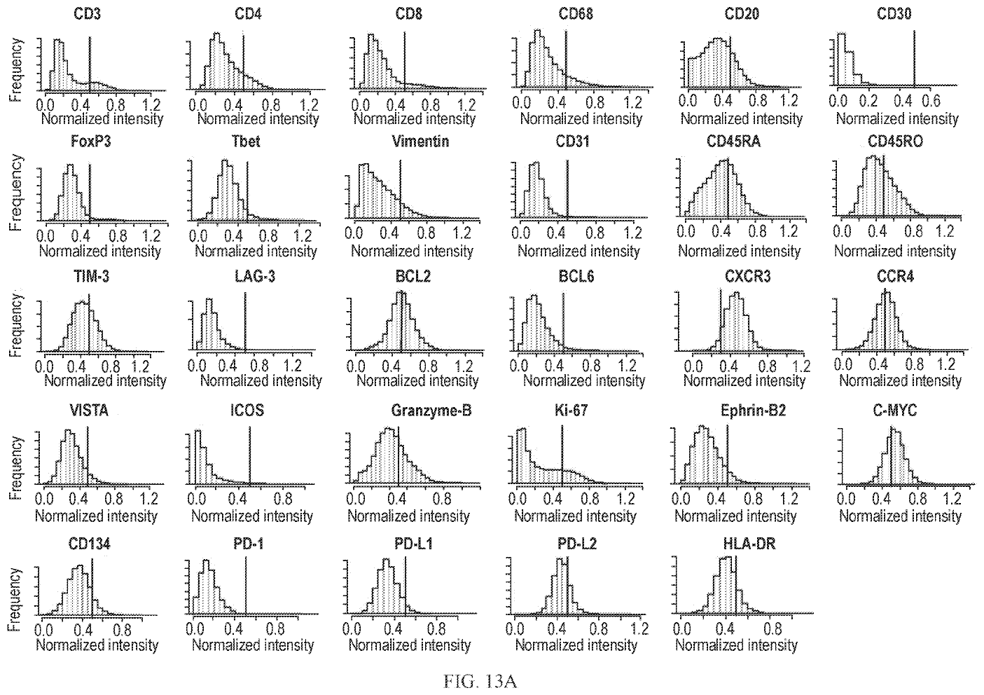

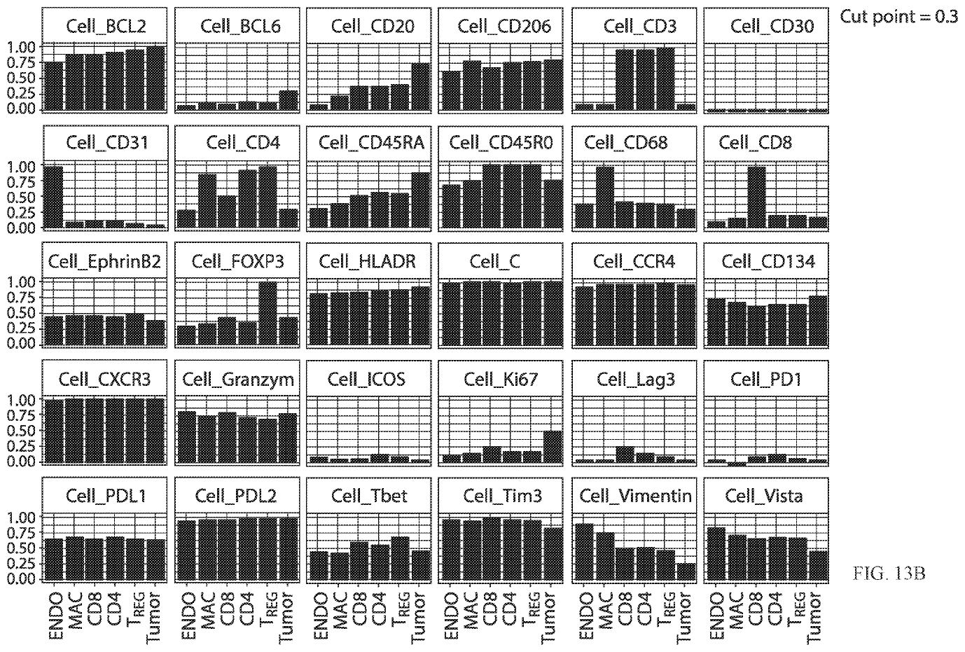

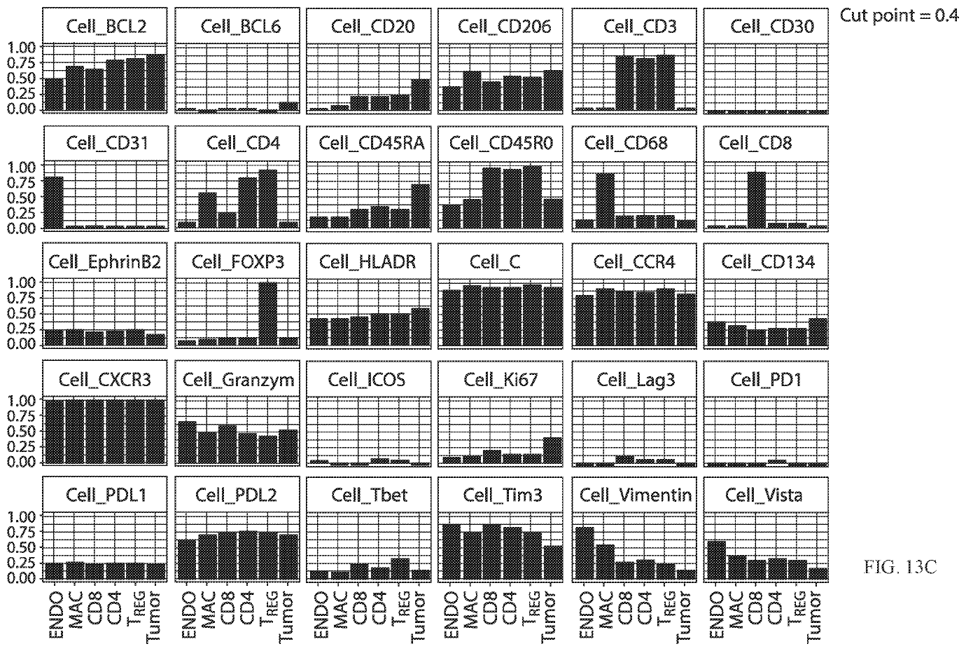

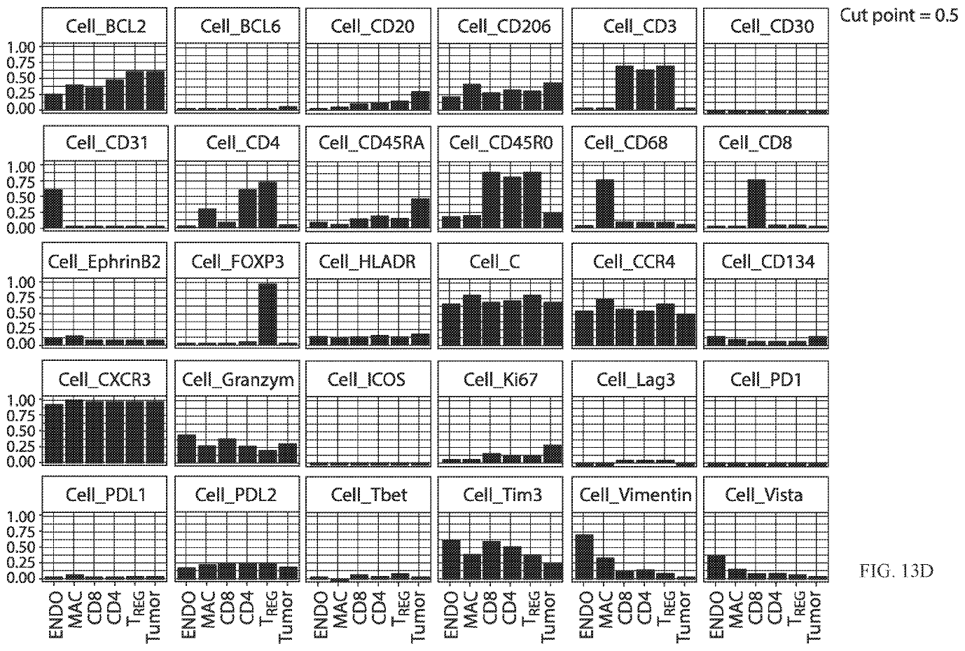

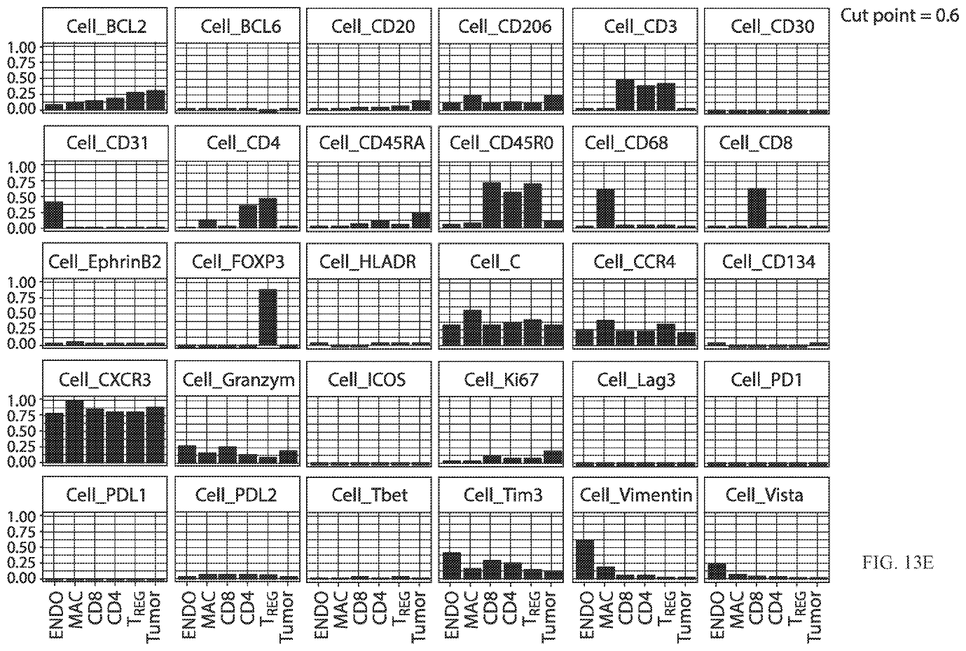

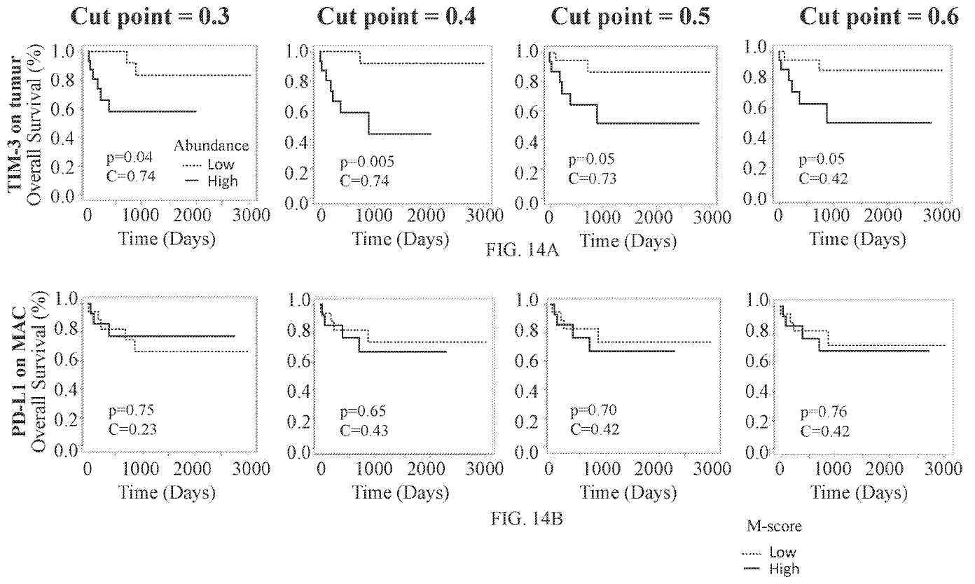

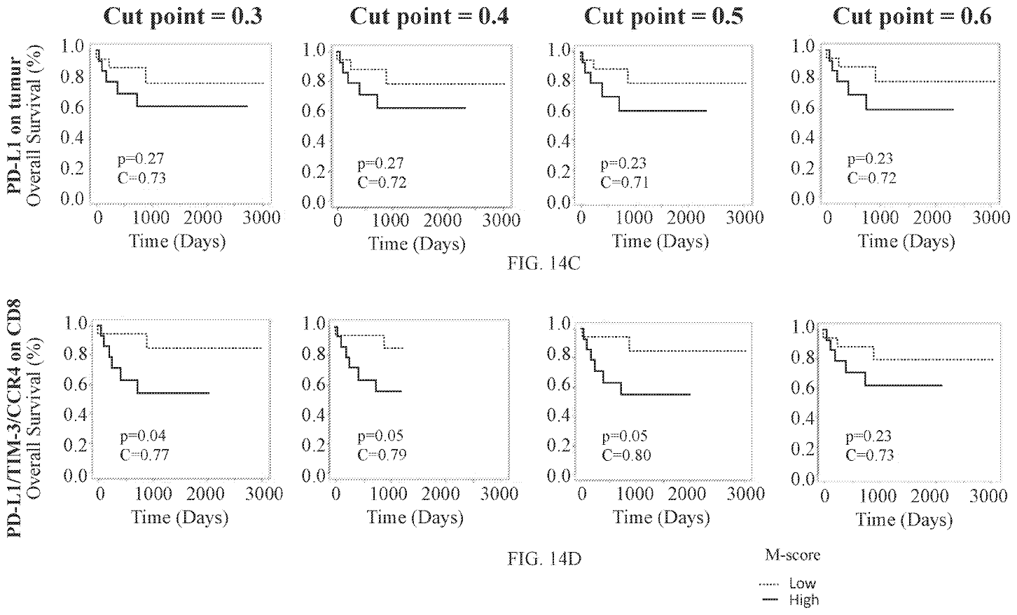

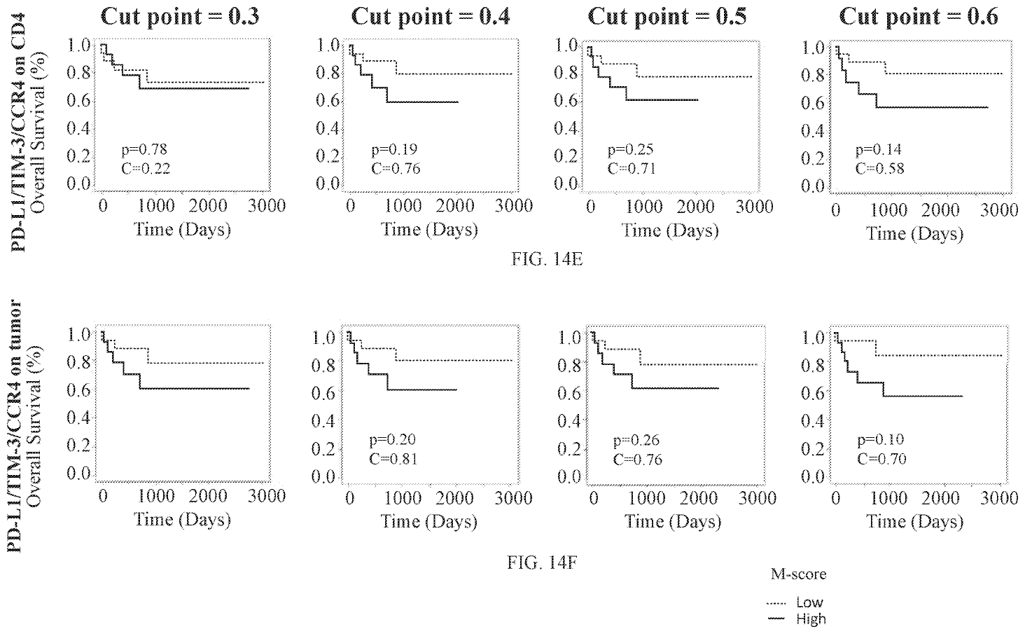

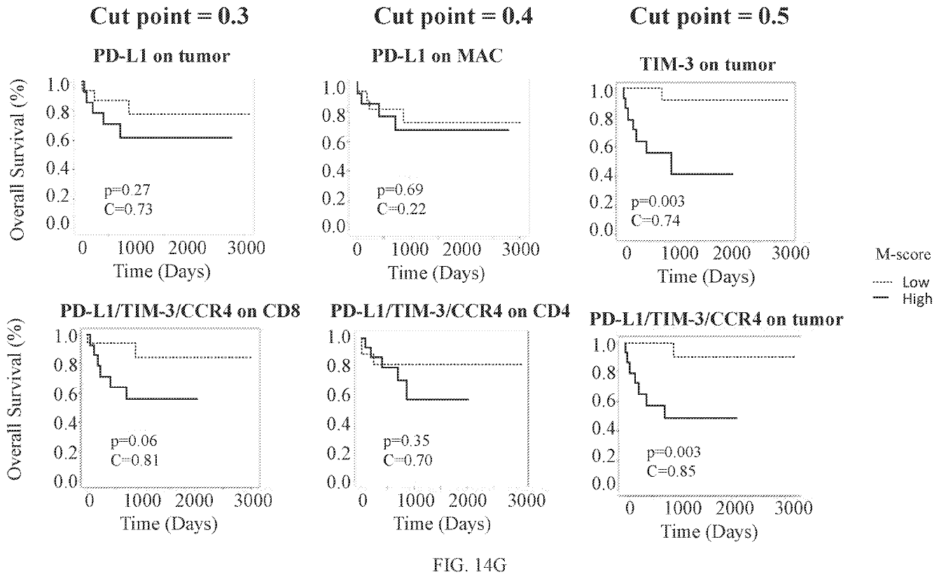

FIG. 13A-FIG. 13E depict in accordance with various embodiments of the invention, distribution of marker intensities and phenotypes at different cut points. Related to FIG. 7A and FIG. 7B. FIG. 13A Distribution of normalized intensity of each marker. Red vertical line indicates 0.5 cut point. FIG. 13B-13E distribution of previously meta-clustered CD4, CD8, T_{REG}, MAC, ENDO and tumor phenotypes expressing each marker at different cut points of 0.3 (FIG. 13B), 0.4 (FIG. 13C), 0.5 (FIG. 13D), and 0.6 (FIG. 13E).

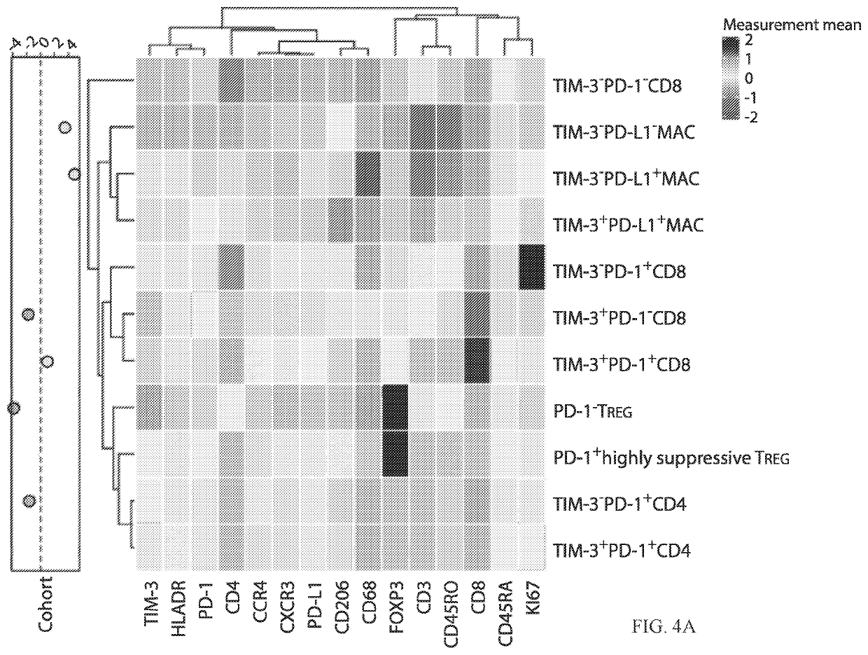

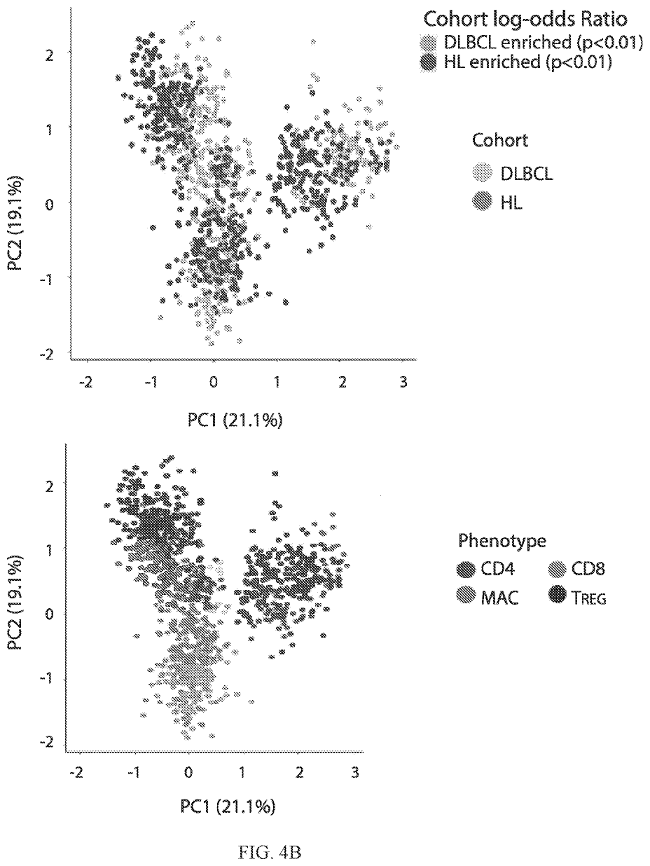

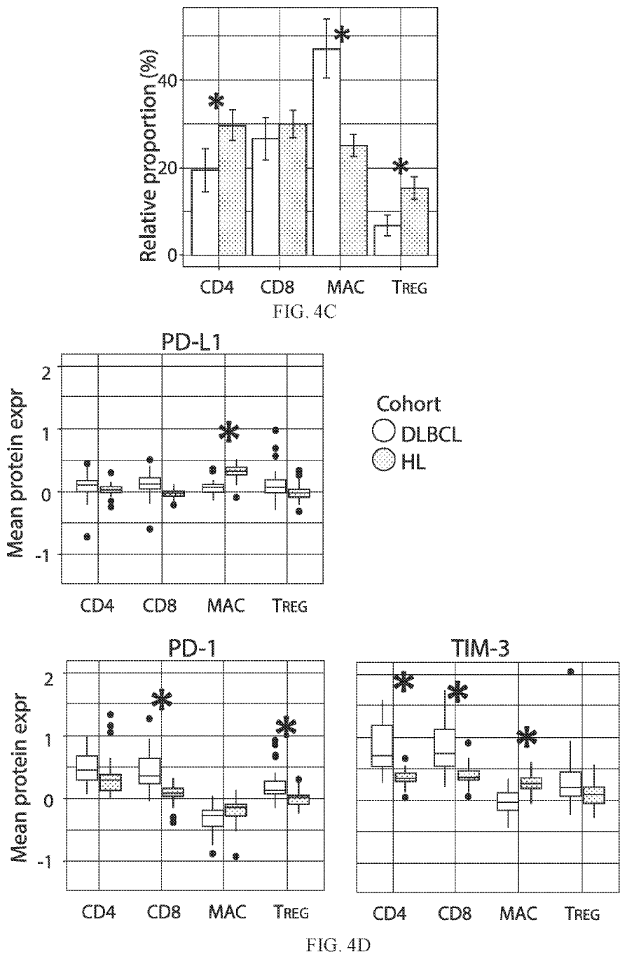

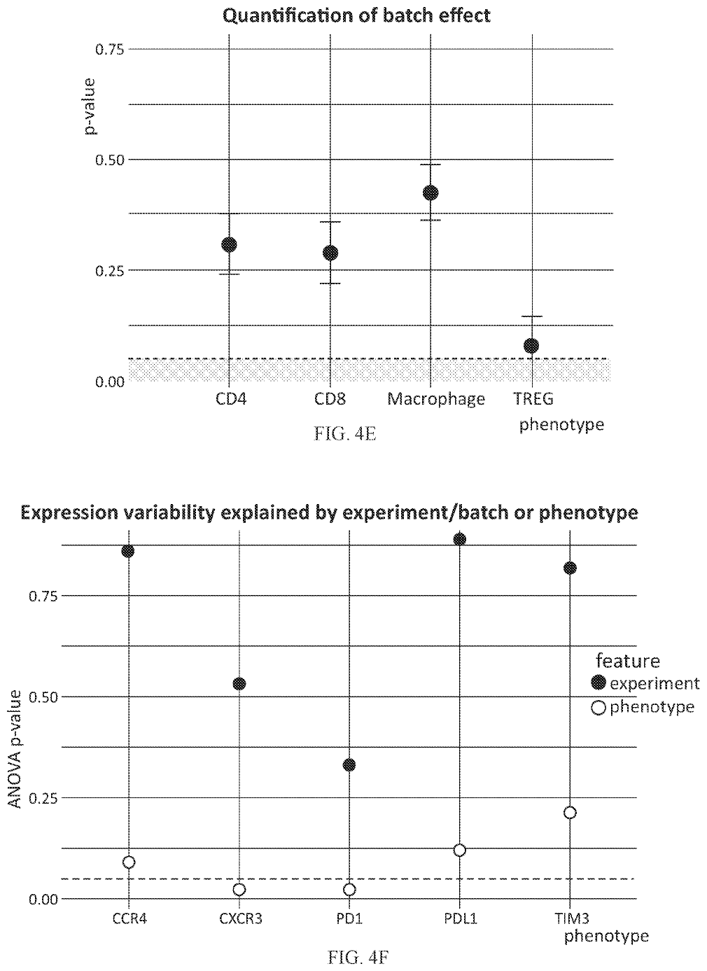

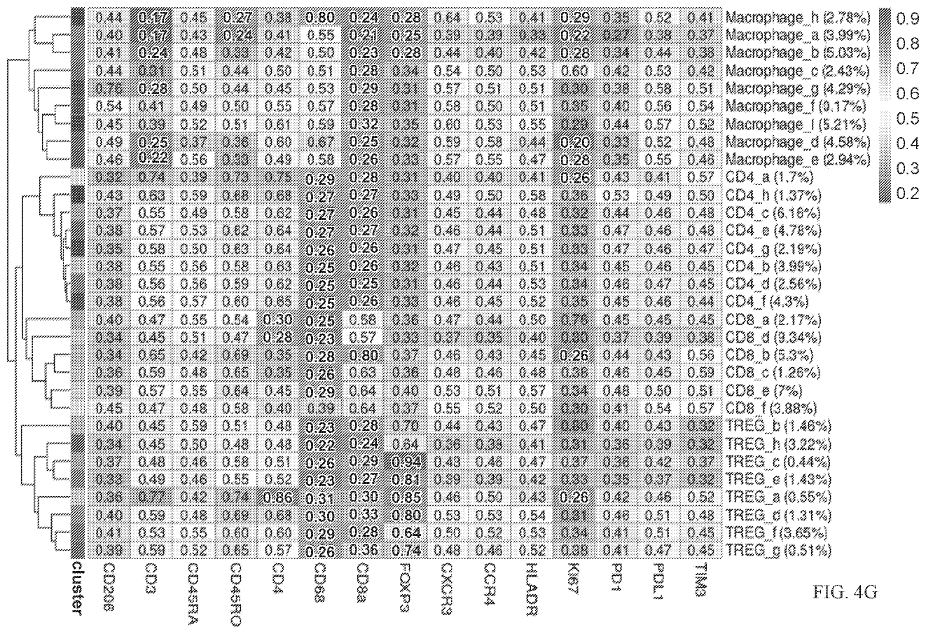

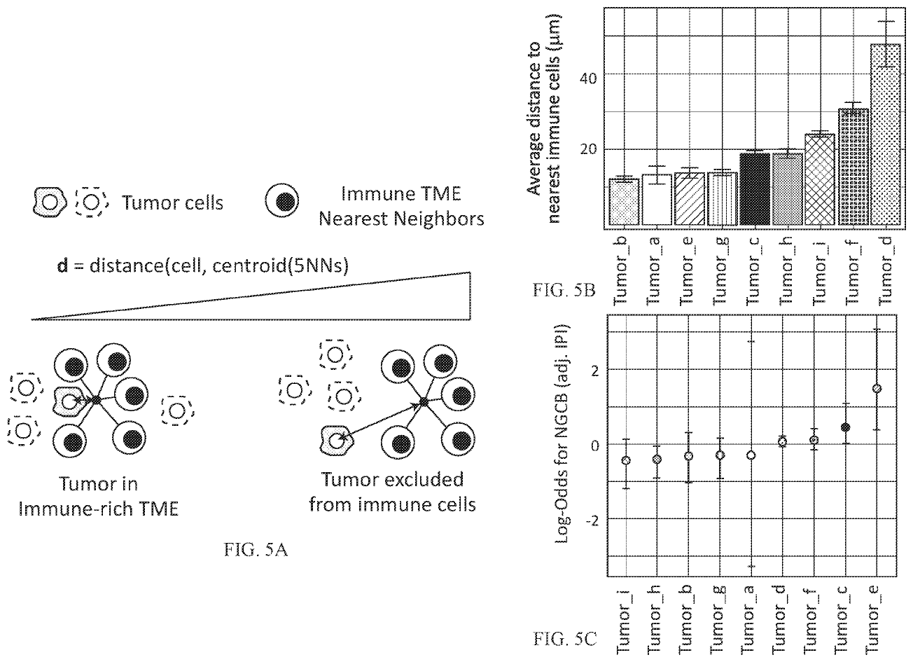

FIG. 14A-FIG. 14G depict in accordance with various embodiments of the invention, survival analyses based on cut points and M-score for phenotypes expressing PD-L1, TIM-3 and/or CCR4. Related to FIG. 7G. FIG. 14A-FIG. 14C the associations between overall survival (OS) and phenotypic abundance follow similar trends across cut points for phenotypes that express single inducible marker: TIM-3 on tumor (FIG. 14A), PD-L1 on MAC (FIG. 14B), PD-L1 on tumor (FIG. 14C). FIG. 14D-FIG. 14F both the LRT p-value and C-index for survival prediction of complex tumor and T cell phenotypes co-expressing PD-L 1/TIM-3/CCR4 fluctuated at different positivity cut points: PD-L1 on tumor (FIG. 14C), PD-L 1/TIM-3/CCR4 on CD8 T cell (FIG. 4D), PD-L1/TIM-3/CCR4 on CD4 T cell (FIG. 4E), and PD-L1/TIM-3/CCR4 on tumor (FIG. 4F). FIG. 14G Survival analyses using M-score show similar association trends for all phenotypes as in cut-point-based analyses. Considering the predictive power of Harrell's C-index from the Cox proportional hazards regression models, the models using M-score show better survival prediction for PD-L1-expressing and PD-L1/TIM-3/CCR4 co-expressing tumor phenotypes and PD-L1/TIM-3/CCR4 co-expressing CD8 phenotype, reflected by higher C-index.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 Jul. 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, systems, articles of manufacture, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

In some embodiments, the numbers expressing quantities of ingredients, properties such as amounts, concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

“Optional” or “optionally” means that the subsequently described circumstance may or may not occur, so that the description includes instances where the circumstance occurs and instances where it does not.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

“Sample” is used herein in its broadest sense. The term “biological sample” as used herein denotes a sample taken or isolated from a biological organism (e.g., a subject). In some embodiments, the sample is a biological sample.

In some embodiments, the sample or biological sample is a tissue sample. Non-limiting examples of tissue samples include diffuse large B cell lymphoma (DLBCL) tissue, Hodgkin's lymphoma tissue, other non-Hodgkin lymphoma tissue, breast cancer tissue, ovarian cancer tissue, prostate cancer tissue, melanoma tissue, and combinations thereof.

In some embodiments, the sample or biological sample is a tumor sample. Non-limiting examples of tumor samples include diffuse large B cell lymphoma (DLBCL) tumor, Hodgkin's lymphoma tumor, other non-Hodgkin lymphoma tumor, breast cancer tumor, ovarian cancer tumor, prostate cancer tumor, melanoma tumor, and combinations thereof.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. In some embodiments, the subject is a human.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age. Thus, adult and newborn subjects, as well as fetuses, are intended to be included within the scope of this term.

The term “cancer” as used herein is not limited to a particular type of cancer. Non-limiting examples of cancer include diffuse large B cell lymphoma (DLBCL), Hodgkin's lymphoma, other non-Hodgkin lymphoma, breast cancer, ovarian cancer, prostate cancer, melanoma and combinations thereof. In some embodiments, the cancer is not a solid cancer; in some embodiments, the cancer refers to a circulating tumor; and in other embodiments, the cancer is a solid cancer. Non-Hodgkin lymphoma includes but is not limited to diffuse large B-cell lymphoma, anaplastic large-cell lymphoma, Burkitt Lymphoma, lymphoblastic lymphoma, mantle cell lymphoma, and peripheral T-cell lymphoma.

The term “cell” or “cells” or “cell type” as used herein is not limited to a particular type of cell or cells. Non-limiting examples of cells include CD8 T cells (T_{CYTO} cells), CD4 T cells (T_{HELPER} cells), regulatory T cells (T_{REG}), tumor cells, macrophages (MAC), endothelial cells (ENDO), and combinations thereof.

The terms “marker” or “biomarker” are used interchangeably herein, and in the context of the present invention includes but is not limited to one or more proteins (including but not limited to hormones, antibodies, enzymes, soluble proteins, cell surface proteins, secretory proteins), gene products, protein fragments, peptides, nucleic acids (including but not limited to DNA, RNA, microRNA, siRNA, shRNA), or lipids.

Non-limiting examples of proteins, or markers or genes encoding these proteins, include BCL-2, BCL-6, CD134, CD183, CD194, CD20, CD206, CD3, CD4, CD30, CD31, CD34, CD4, CD45RA, CD45RO, CD68, CD8a (CD8), c-Myc p67 (C-MYC), CCR4, CXCR3, Ephrin B2, FoxP3, Granzyme B, Histone 3, HLA-DR, ICOS, Ki-67, LAG-3, PD-1, PD-L1, PD-L2, pStat3, Tbet, TIM-3, Vimentin, Vista, and combinations thereof.

Non-limiting examples of markers include phenotypic markers, inducible markers, and combinations thereof. Phenotypic markers are markers that define cell types or lineages. Inducible markers are markers that define cell states or are inducible.

In some embodiments, the number of markers (e.g., number of markers in the sample) is up to 34. In some embodiments, the number of markers is 1-34, 1-33, 1-32, 1-31, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, 1-2, or 1. In some embodiments, the number of markers is up to 34, up to 33, up to 32, up to 31, up to 30, up to 29, up to 28, up to 27, up to 26, up to 25, up to 24, up to 23, up to 22, up to 21, up to 20, up to 19, up to 18, up to 17, up to 16, up to 15, up to 14, up to 13, up to 12, up to 10, up to 9, up to 8, up to 7, up to 6, up to 5, up to 4, up to 3, up to 2, or up to 1. In some embodiments the number of markers is 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1. In some embodiments, the markers include those listed in Table 2.

In some embodiments, the number of markers (e.g., number of markers in the sample) that are measured or detected, (e.g., in one measurement) is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 markers. In some embodiments, the number of markers is 1-75. In some embodiments, the methods include simultaneously measuring or detecting up to 34 markers. In some embodiments, the methods include simultaneously measuring or detecting 14, 15, 16, 17, 18, 19, 20, 21, 22, 23, 24, 25, 26, 27, 28, 29, 30, 31, 32, 33 or 34 markers.

The term “index cell” as used herein means a cell that is used as a reference in which to establish a relation (e.g., a distance) between one or more other cells.

The term “double-hit lymphoma” generally refers to the presence of both the MYC and BCL2 rearrangements, which is a phenotype that is very proliferative, drug-resistant, and often associated with a poor prognosis. In t(8;14) translocation, MYC is rearranged; and in t(14;18) translocation, BCL2 is rearranged. Another variant of double-hit lymphoma is co-rearrangement of MYC and the BCL6 gene. When all 3 genes—BCL2, MYC, and BCL6-are simultaneously rearranged, the phenotype is termed “triple-hit lymphoma.” Hence in various embodiments, performing gene expression profiling to identify double- or triple-hit lymphoma includes sequencing at least BCL2, MYC, and/or BCL6 genes to identify any rearrangement.

The term “double-expressor” refers to a phenotype with overexpression of MYC and BCL2 genes at a protein level, without the genetic rearrangements. Dual-expresser protein, or double protein, refers to immunohistochemical detection of MYC and BCL2 overexpression. This profile was referred to as the “double-expressor” phenotype in DLBCL in the revised World Health Organization (WHO) classification of lymphoid neoplasms, which was published in 2016 in Blood.

The identification of the immune cells comprises or consists of one or more of identifying of one or more immune cell types that boost an immune reaction (e.g. macrophages, cytotoxic T-cells, memory cells, B cells, and T-helper cells), identifying immune cells of an immune cell type that suppresses or downregulates an immune reaction (e.g. regulatory T-cells), as well as ignoring (“filtering out”) one or more types of immune cells.

Here we report our efforts to characterize the TME in DLBCL, including the cell types, frequency, functional state and spatial topology, using imaging mass cytometry (IMC). Key differences are identified between the Hodgkin lymphoma and DLBCL TME components that may explain their differential responses to immune check point inhibitor (ICI) therapy. We further performed spatial analysis of the DLBCL tumor architecture to reveal immunologic sub-regions of tumor immune interaction at single cell resolution.

Non-limiting examples of labels include antibody label, isotope label, fluorescent label, fluorochrome label, a fluorophore label, and combinations thereof.

Non-limiting examples of isotope labels include metal isotopes. Non-limiting metal isotopes include 142Nd, 143Nd, 144Nd, 145Nd, 146Nd, 147Sm, 148Nd, 149Sm, 150Nd, 151Eu, 152Sm, 153Eu, 154Sm, 155Gd, 156Gd, 158Gd, 159Tb, 160Gd, 161Gd, 162Dy, 163Dy, 164Dy, 166Er, 167Er, 168Er, 169Tm, 170Er, 172Yb, 173Yb, 174Yb, 175Lu, 176Yb and combinations thereof.

In various embodiments, non-limiting examples of phenotypes are selected from BCL-2, BCL-6, CD134, CD183, CD194, CD20, CD206, CD3, CD4, CD30, CD31, CD34, CD4, CD45RA, CD45RO, CD68, CD8a (CD8), c-Myc p67 (C-MYC), CCR4, CXCR3, Ephrin B2, FoxP3, Granzyme B, Histone 3, HLA-DR, ICOS, Ki-67, LAG-3, PD-1, PD-L1, PD-L2, pStat3, Tbet, TIM-3, Vimentin, Vista, and combinations thereof. In various embodiments, methods of performing complex spatial analysis of a tissue sample are provided, comprising: identifying two or more different phenotypic clusters of cells in a tissue sample or an image of the tissue sample, wherein each phenotypic cluster of cells is identified based on two or more closest neighboring cells of a same phenotype to an index cell; and determining an average marker intensity of each phenotypic cluster of cells.

In various embodiments, the methods further comprises comparing the average marker intensity of each phenotypic cluster of cells to an average marker intensity of another phenotypic cluster of cells. In some embodiments, the comparison is between a phenotypic cluster that is associated with responsiveness or a high likelihood of responsiveness to a therapy and a phenotypic cluster that is associated with unresponsiveness or a high likelihood of unresponsiveness to the therapy.

In some embodiments, identifying phenotypes (or phenotypic clusters) or classifying subtypes for a cancer/tumor includes one or more of detecting the RNA expression levels of a plurality of genes in cancer tissue samples to generate a (first) gene expression profile, detecting cDNA expression levels of the genes in cancer tissue samples after performing reverse-transcriptase polymerase chain reaction to generate a (second) gene expression profile, comparing the gene expression profiles with one another (e.g., comparing that of a known/reference cancer sample to that of a test tissue sample) or against known or deposited gene expression data in database, and constructing centroids and/or calculating distance utilizing a nearest centroid algorithm, thereby assigning the test sample to one phenotype or subtype. Further description of classifying a cancer subtype suitable for identifying tumor phenotypes is seen in U.S. Pat. No. 9,631,239, which is herein incorporated by reference.

In some embodiments, the method further comprises first using imaging mass cytometry to generate an image of the tissue sample. In some embodiments, the tissue sample is first subjected to mass spectrometry imaging to generate an image of the tissue sample. In various embodiments, the tissue sample is imaged with imaging mass cytometry, which allows for single-cell measurements and multiplexed quantitative detection of molecular targets or antigen expression in single cells within tissue samples. In some embodiments, the tissue samples are imaged with other techniques such as immunofluorescence microscopy and laser ablation-inductively coupled plasma-mass spectrometry (LA-ICP-MS). Generally immunofluorescence microscopy are useful for imaging down to nanometer resolutions, but is limited in practice to measuring seven or fewer targets simultaneously; whereas LA-ICP-MS offers highly multiplexed quantitative analysis in single cells, but it typically lacks the resolution necessary for the imaging of single cells within tissue samples. In Various embodiments of the methods, an imaging step of the tissue sample includes, or the tissue sample is imaged in a process including, (a) labeling a plurality of different target molecules in the tissue sample with a plurality of different labeling agents (or molecular tags), to provide a labeled tissue sample, (b) subjecting multiple cells of the labeled tissue sample to laser ablation, typically with a subcellular resolution, to form a plurality of plumes or where molecular tags are atomized and/or ionized (thereby releasing or partially releasing a reporter molecule), and (c) subjecting plumes (or released reporter molecules) to inductively coupled plasma mass spectrometry, whereby detection of labeling atoms (or reporter molecules) in the plumes permits construction of an image of the tissue sample. Imaging mass cytometry is further described in WO2015128490 and US20160139141, which are incorporated herein by reference.

In some embodiments, the methods further include identification of malignant B cells, identification of endothelial cells, and identification of other cells belonging to the tumor microenvironment (TME) in the tissue sample. In further embodiments, the methods include selecting the cells belong to TME and/or the malignant B-cells for performing complex spatial analysis.

In some embodiments, the method further comprises calculating a centroid location of each phenotypic cluster of cells by averaging the X,Y coordinates of the two or more neighboring cells of the same phenotype. In some embodiments, the centroid location is identified based on a phenotypic cluster that is nearest or closest to the index cell.

In further embodiments, the distance is computed between the centroid location of a phenotypic cluster and an index cell. In some embodiments, the distance from the index cell to the centroid is less than 200 μm, 150 μm, 100 μm or 50 μm. In some embodiments, the distance from the index cell to the centroid is about 30 μm, 25 μm, 20 μm, or 15 μm, i.e., a hyper-local microenvironment within 2-3 cell diameters of a tumor cell or a phenotypic cluster of tumor cells.

In some embodiments, the index cell is a tumor cell, and the marker comprises one or more immune cell markers. In other embodiments, the index cell is an immune cell, and the marker comprises one or more tumor cell markers. In some embodiments, the distance or proximity between the tumor cells and the immune cells indicates whether a sufficiently large number of immune-promoting immune cells such as cytotoxic T cells, B cells, memory cells, T-helper cells and/or macrophages are within an immunologically effective distance from the tumor cells. In several instances, an “immunologically effective distance” is a distance between an immune cell and its nearest tumor cell which is sufficiently small to allow for the killing of said tumor cell by said immune cell.

Further embodiments of the methods comprise identifying a first, tumor-core-immune-desert zone which comprises a phenotypic cluster whose centroid distance to the index cell is no less than a first threshold or the farthest in all clusters, or within which immune activity is lowest in all clusters as characterized by substantially absent of proliferative CD8+ T cells, macrophages or T_{REG} cells; identifying a second, tumor-dispersed-immune-activation zone which comprises a phenotypic cluster whose centroid distance to the index cell is no greater than a second threshold or the shortest in all clusters, or within which immune activity is activated as characterized by substantial presence of proliferative CD8+ T cells, macrophages and T_{REG} cells; and/or identifying a third, tumor-immune-interface zone which comprises a phenotypic cluster whose centroid distance to the index cell is between the first threshold and the second threshold or between the first zone and the second zone, or within which immune activity is suppressed as characterized by substantial presence of exhausted CD8+ T cells and suppressive T_{REG} cells.

In further embodiments, the first threshold is 40 μm, 41 μm, 42 μm, 43 μm, 44 μm, 45 μm, 46 μm or 47 μm, and the second threshold is 12 μm, 13 μm, 14 μm, 15 μm, 16 μm, 17 μm, 18 μm, 19 μm, or 20 μm; or the tumor-core-immune-desert zone includes a volume that is within a radius of 12 μm, 13 μm, 14 μm, 15 μm, 16 μm, 17 μm, 18 μm, 19 μm, or 20 μm from the core of tumor, and the tumor-immune interface ends approximately 40 μm, 41 μm, 42 μm, 43 μm, 44 μm, 45 μm, 46 μm or 47 μm from the core of the tumor.

In various embodiments, the methods include classifying immune cells (e.g., CD4 T cells; CD8 T cells; macrophages) into one(s) within the tumor-core-immune-desert zone, one(s) within the tumor-dispersed-immune-activation zone, or one(s) within the tumor-immune-interface zone.

In some embodiments, the method further comprises standardizing the distances to the centroids by dividing each centroid distance by the total number of cells of the corresponding phenotype. In some embodiments, the method further comprises scaling the standardized distances by the cohort average abundance of the corresponding phenotype. In some embodiments, the method further comprises mean centering and scaling the standardized distances by their standard deviation to derive Z-scores. In some embodiments, the method further comprises clustering the Z-scores of the distances. In some embodiments, the method further comprises meta-clustering by using the centroid distances per individual case phenotypic clusters. In some embodiments, the method further comprises filtering out distant interactions.

In some embodiments, the two or more closest neighboring cells is 5 neighboring cells. In some embodiments, the two or more closest neighboring cells is 3, 4, 5, 6, 7, 8, 9 or 10 neighboring cells. In some embodiments, the two or more closest neighboring cells is 5-10, 11-15, 16-20, 21-25, or 26-30 neighboring cells. In some embodiments, the method comprises identifying 3, 4, 5, 6, 7, 8, 9, or 10 different phenotypic clusters of cells. In some embodiments, the method comprises identifying 5-10, 11-15, 16-20, 21-25, 26-30, 31-35, 36-40, 41-45, or 46-50 different phenotypic clusters of cells. In some embodiments, cells are labeled with a label. In some embodiments, the label is selected from the group consisting of antibody label, isotope label, fluorescent label, fluorochrome label, a fluorophore label, and combinations thereof.

In various embodiments, the present invention provides a method for profiling a tumor microenvironment, comprising: performing imaging mass cytometry (IMC) to obtain imaging mass cytometry data of a tumor sample from a subject in need thereof; and performing a method of any of the present invention to obtain information about the cells in the tumor sample. In some embodiments, a panel of the 32 markers according to Table 2 are measured or detected to generate information about the cells in the tumor sample. In some embodiments, a panel of markers indicating of exhaustion state (e.g., TIM-3, LAG-3, PD-1) and markers indicating proliferation (e.g., Ki67) are measured.

Further embodiments of the methods provide performing cell-of-origin analysis and/or mutation screen on the tissue sample, which in some instances is in addition to performing the IMC.

M-Score Analysis

To describe the expression of proteins, we created a novel scoring metric, an intensity weighted abundance score, or M-score, for identifying complex cellular phenotypes associated with survival. Using the M-score, we identify a CCR4+Tim3+PDL1+ tumor phenotype that predicts outcomes better than the gold standard clinical predictor used for lymphoma, the international prognostic index (IPI). To characterize the patterns of spatial interaction in the TME, we developed a novel unsupervised multivariate model to construct spatial meta-clusters based on average distances from CD8 to the centroids of 5 nearest neighbors. Spatial analysis revealed CD8 T cell neighborhoods, or “micro-regions,” that were associated with responses to therapy. Spatial clusters termed “hazardous” had almost 3 times higher odds of being identified in refractory cases compared to clusters termed “protective”. In the “protective” spatial neighborhoods, we observed the presence of activated CD8, Th1-like CD4, and less suppressive T_{REG} phenotypes, with the opposite in “hazardous” areas, allowing for functional annotation of cells within each cluster.

Previous efforts to create a multiplexed understanding of the tumor microenvironment revealed a non-random structure or architecture in the immune microenvironment, however we believe this is the first paper to create functional clusters of immune response based solely on their spatial organization, to organize these clusters based on clinical response to treatment and then use information about differentially expressed markers to gain insights in tumor immunology. Told from the point of view of the CD8 T cell, we can understand the dialectical relationships between immune cells and tumor cells in their micro-environments, where each cell modulates the phenotype of other cells, and is simultaneously shaped by its environment, thus creating the structures of immune organization we observe.

When considered in the context of previous studies of the TME in lymphoma, our results can shed new insights into those observations. For example, Ansell et al. reported that in DLBCL patients treated with CHOP, the presence of >20% of CD4 T cells in pre-treatment biopsies was significantly associated with longer recurrence-free and overall survival. Similarly, Keane and colleagues found that 23% or more of CD4 T cells in diagnostic lymphoma samples independently predicted better event-free and overall survival in a cohort of 122 DLBCL patients treated with R-CHOP. Consistent with these observations, our results demonstrate that favorable CD8 neighborhoods tended to have closer interactions with CD4 cells, and that in the most favorable clusters, CD4 cells showed increased Th1 polarization.

Given the clinical failure of PD1/PDL1 inhibitors in DLBCL, there is much interest in identifying novel IO targets for DLBCL. In this study, we identified a subset of tumor cells co-expressing PD-L1/TIM-3/CCR4 to be significantly enriched in refractory cases. Moreover, this complex tumor phenotype predicted overall survival better than IPI, indicating that R/R DLBCL patients could potentially benefit from combination therapy targeting PD-L1, TIM-3 and CCR4.

While we only examine a relatively small part of the tumor area (approximately lmm²), within that small area, we are area able to demonstrate tremendous spatial and functional heterogeneity and describe a complex tissue architecture of immune response and suppression that corresponds to clinical outcomes. Optimal sample size will also depend on the specific tumor type and will need to be determined empirically. In our data, seven cases had at least one duplicate core on the TMA. Preliminary analysis by PCA indicated that most duplicates clustered together (FIG. 11C. This would indicate that small sample areas may be adequate to understand the complexity of tumor immune responses. This is important because on-treatment and progression biopsies, which are critical to understand the kinetics of immune response and failure to immune-oncology agents are often obtained as core needle biopsies, which are much small than the diagnostic tissue samples.

Our approach and spatial analysis framework can be applied to patients treated with newer immuno-oncology agents, and could be tremendously helpful in understanding why single agent PD-1/PD-L1 inhibitors have such poor responses in DLBCL. To that point we noted with interest that PD-1 expression both on CD8 and on CD4 T cells tended to be lower in the more hazardous spatial clusters. This could indicate that targeting PD-1 would shift the TME from a more protective to a more hazardous phenotype, which may explain the poor results with single agent PD-1/PD-L1 inhibitors in DLBCL. Most importantly, the multiplex analysis provided by IMC could help guide the next generation of combination therapies with newer agents targeting CCR4, TIM-3 and others. Recently, IMC has been successfully used to study patients treated with chimeric antigen receptor (CAR)-T cells (unpublished observations) and could be used to understand mechanism of resistance and clinical failure for cellular therapies. In summary, IMC analysis of lymphoma reveals phenotypic and spatial structure in the TME that gives new insights to tumor immunology.

In various embodiments, the present invention provides a method for calculating an intensity-weighted abundance score of a marker in a tissue sample, comprising: determining a normalized marker intensity across all cells in the tissue sample or an image of the tissue sample; dividing the tissue sample or the image of the tissue sample into a plurality of blocks based on the quantiles of the total dynamic range of the tissue sample or the image of the tissue sample; assigning an average intensity of cells in each block of the plurality of blocks; counting the number of cells in each block of the plurality of blocks; and determining the intensity-weighted abundance score of the marker by a linear combination of the number of cells in each block and the average intensity of the respective block. In some embodiments, the method further comprises first using imaging mass cytometry to generate an image of the tissue sample. In some embodiments, the tissue sample is first subjected to mass spectrometry imaging to generate an image of the tissue sample. In some embodiments, the tissue sample is first subjected to mass spectrometry imaging to generate an image of the tissue sample. In some embodiments, the plurality of blocks is 10 blocks. In some embodiments, the plurality of blocks is 2-5, 6-10, 10-15, 16-20 blocks. In some embodiments, an intensity-weighted abundance score is calculated for more than one marker in the tissue sample. In some embodiments, an intensity-weighted abundance score is calculated for 2, 3, 4, 5, 6, 7, 8, 9, 10, 11, 12, 13, 14, 15, 16, 17, 18, 19, 20, 21, 22, 23, 24, 25, 26, 2, 7 28, 29, 30, 31, 32, 33, 34, 35, 36, 37, 38, 39 or 40 markers in the tissue sample. In some embodiments, an intensity-weighted abundance score is calculated for up to 34 markers in the tissue sample. In some embodiments, an intensity-weighted abundance score is calculated for 36-40, 41-45, 46-50, 51-55, 56-60, 61-65, 66-70, or 71-75 markers in the tissue sample. In some embodiments, cells are labeled with a label. In some embodiments, the label is selected from the group consisting of antibody label, isotope label, fluorescent label, fluorochrome label, a fluorophore label, and combinations thereof.

In various embodiments, the present invention provides a method for determining a patient level summary, comprising: performing a method of the present invention; and taking a weighted sum of the average intensity for each block and the number of cells within each block.

In some embodiments, the cut point is 0.05-0.85. In some embodiments, the cut point is 0.05-0.1, 0.1-0.2, 0.2-0.3, 0.3-0.4, 0.4-0.5, 0.5-0.6, 0.6-0.7, or 0.7-0.85. In some embodiments, the cut point is 0.3. In some embodiments, the cut point is 0.4. In some embodiments, the cut point is 0.5. In some embodiments, the cut point is 0.6. In some embodiments, the cut point is 0.7. In some embodiments, the cut point is 0.8.

M-Score Computation
1. Input: X: Identify marker to summarize, expression matrix (rows cells, columns are proteins), the number of blocks used for summary.
2. Cut: uniformly cut the dynamic range of the intensity values into the blocks:
a. Using Hmisc::cut2 function, we assign each object into 1 block and identify the average value per block.
b. For each subject (i)
i. For each unique block(j) identified compute the total objects corresponding to a given block.
1. Total<−0
2. If object_i belongs to block_j, Total<−Total+1.
3. Abundance(I,j)<−Total/total cell objects for subject (absolute)
4. Return absolute abundance of cell objects in a given block relative to subject.
c. For each subject,
i. For each block k; initialize subject(j) Mscore.
ii. Subject(j,k) M score=abundance block(j,k)*average block(j,k)
iii. END multiplication over block
iv. Subject(j) M-score=sum k M-score weights for subject(j,k) M-score.
v. Return subject(j) M-score.

Spatial Analysis algorithm (RICO):
1. Input: data matrix, rows as objects and columns include proteins, subject ID and X, Y positional information. Functional classification of each cell is also helpful, but not required. Index/reference functional class is required. Also need a list of protein(p) of interest to summarize in the ecosystem.
2. For each subject(j)
a. For each object functional classification(k)
i. Compute the distance matrix from index cell(i) to object classification(k).
ii. For each index cell(i), identify the indices to the closet N objects from cell(i) to classification(k). (store indices into object ID).
1. Find the centroid by averaging the coordinates of the N objects classification(k; ID).
2. Compute the distance from index cell(i) coordinates and the centroid of the object classification(k, ID)
3. Identify the average protein(p) intensity of objects classification(k, ID)
4. Store the data into subject(j) matrix of cell(j,I, k:p) and column representing average protein intensity k(p) of object classification(k, ID).
iii. Return a data matrix with rows equal to total cells of index type(i), across all patients, columns corresponding to each k classifications per protein of interest (p), and distance from cell(i) to centroid of object (k; ID).
b. Compute Phenograph clusters using data matrix centroid vectors. The rows are all the index types for cell(i), and the columns are the centroid distances for each classification type(k).
i. Normalization: let_i be the reference index cell, and let k be the functional classification class. We normalize the distances from cell(i) to the class type k to control for the abundances of the secondary type.
1. For each functional class K:
2. Normalized distance from cell(I,k): average total abundance of object k across all subject*(distance from cell(i) to centroid of object(k))/total abundance of object(k)).
3. Return: a normalized vector of scaled distances for all cell(i).
ii. Standardize the distance by row scaling.
iii. Run Phenograph algorithm on the row scaled normalized distances.
iv. Adjoin the distance network classification to the data matrix in 2.

Spatial Analysis Clusters to Compute Odds Risk and Survival Data:
1. Using the data matrix from the Spatial Analysis (RICO) algorithm, we have patient information such as treatment response, or other clinical variables. The data matrix from the RICO algorithm also contains the network assignment for each cell(j,i) corresponding to subject(j) and index cell(i).
a. Odds risk: tabulate a nXm frequency table for each patient clinical variable and the number of cells in a given network.
b. OR: Group(A)/total(A)/group(B)/total(B)

UMAP Algorithm on the Distances:
2. tSNE and uMAP algorithms can be applied to the distances to summarize the distance clustering for all patients.

Marginal Distances for Intra-Adjacent-Distal Interactions:
3. For univariate distance interactions, look at one column from the RICO data matrix which has information for all cell(i) and distances to centroids(k); for fixed k.
a. Split the distances into tertiles, for each tertile assign classification (intra/adjacent/distal) for a categorical analysis.

### Prognosis

Various embodiments of provide a method of predicting response to an antitumor therapy in a subject having a cancer, which comprises performing a complex spatial analysis on a tissue sample of the subject, wherein the marker intensity of at least one of the phenotypic cluster in indicative of response to the antitumor therapy.

In some embodiment, the antitumor therapy includes an immune checkpoint inhibitor therapy. In some embodiments, the antitumor therapy includes a standard-of-care treatment.

In some embodiments, the method further comprises using the information to provide a prognosis for survival of the subject.

In some embodiments, the method further comprises using the information to predict or monitor an outcome of a cancer treatment. In some embodiments, the complex spatial analysis is performed from a tissue sample collected from a subject after initiation of an antitumor therapy. In some embodiments, the prognosis is based on performing the complex spatial analysis on a tissue sample collected from a subject having received a therapy (e.g., standard-of-care treatment, R-CHOP), or 1 week, 2 weeks, 3 weeks, 1 month, 2 months, or 3 months after initiation of a therapy. In some embodiments, the complex spatial analysis is performed from a tissue sample collected from a subject before initiation of a therapy.

In some embodiments, a method of providing prognosis or predicting response to a therapy in a DLBCL-inflicted patient comprises measuring the expression level, or detecting the presence, of a panel of biomarkers including PD-L1, TIM-3 and CCR4 with the tumor tissue sample of the patient, wherein the presence or expression of the combination of PD-L1, TIM-3 and CCR4 indicates an ineffective response to the therapy or poor survival prognosis. In further embodiments, the absence of the combination of PD-L1, TIM-3 and CCR4 indicates an effective response to the therapy or acceptable survival prognosis.

In some embodiments, identification of presence of suppressive T_{REG} population in the tumor microenvironment in the tumor tissue sample is predicative of a high likelihood of unresponsiveness or ineffective response of the subject to chemotherapy.

In some embodiments of the methods, a largest tumor cell cluster (e.g., more than 50%, 40%, or 60% in cell number in the cluster, or having the highest number of cells in the all clusters) which lacks PD-L1 expression is predicative of a high likelihood of responsiveness or effective response to chemotherapy. In some embodiments of the methods, a largest tumor cell cluster which has high PD-L1 expression or which has Ki67 expression and CCR4 expression and low PD-L1 expression is indicative of a high likelihood of unresponsiveness or ineffective response or refractory to chemotherapy.

In some embodiments of the methods, the presence of PD-L1⁺M2 macrophages, or an increased presence of PD-L1⁺M2 macrophages (relative to presence of T_{REG}), in the tumor microenvironment (e.g., within 30 μm, 20 μm, 40 μm, 50 μm or 60 μm of the tumor cell or a cluster of tumor cells) in indicative of unresponsiveness, ineffective response or refractory to chemotherapy, or a high likelihood thereof.

In some embodiments of the methods, an increased presence of CD4+ T cells within the tumor microenvironment is indicative of responsiveness, effective response or complete responder to chemotherapy.

In some embodiments of the methods, an increased presence of TIM-3⁻PD-L1⁺ macrophages and/or an increased presence of TIM-3′T cells within the tumor microenvironment (to malignant B-cell tumor), is indicative of unresponsiveness, ineffective response or refractory to an immunotherapy drug that targets PD-1, or a high likelihood thereof.

### Treatment

In some embodiments, the method further comprises using the information to determine a treatment/therapy for the subject. In some embodiments, the method further comprises administering a treatment/therapy to the subject. In some embodiments, the treatment is a combination therapy. In some embodiments, the subject received a treatment for a cancer. In some embodiments, the subject has a cancer selected from the group consisting of diffuse large B cell lymphoma (DLBCL), Hodgkin's lymphoma, breast cancer, ovarian cancer, prostate cancer, melanoma, and combinations thereof.

Non-limiting examples of a treatment (e.g., a treatment for cancer) include surgery, chemotherapy, radiation therapy, thermotherapy, immunotherapy, hormone therapy, laser therapy, biotherapy, anti-angiogenic therapy, photodynamic therapy, and combinations thereof.

Non-limiting examples of immune checkpoint inhibitors as a therapy include one or more immunotherapy drugs that work by blocking checkpoint proteins from binding with their partner proteins, for example an immunotherapy drug that targets PD-1 (e.g., an anti-PD1 antibody, pembrolizumab, nivolumab, cemiplimab), a drug that targets PD-L1 (e.g., an anti-PDL1 antibody, atezolizumab, avelumab, durvalumab), a drug that targets TIM-3 (e.g., an anti-TIM-3 antibody, LY3321367), a drug that targets CTLA-4 (e.g., ipilimumab), and, and a drug that targets LAG-3 (e.g., REGN3767, BMS-986016). In some embodiments, a therapy includes a drug that targets CCR4 (e.g., an anti-CCR4 antibody, mogamulizumab). In some embodiments, a therapy includes a drug that targets TIM-3 in combination with a drug that targets CCR4. In some embodiments, the subject is directed to a therapy that involves an anti-TIM-3 antibody, following complex spatial analysis of the tumor tissue sample of the subject. In some embodiments, the subject is directed to a therapy that involves an anti-TIM-3 antibody in combination (sequentially administered, concurrently administered, or pre-mixed) with a drug that targets PD-1 or PD-L1. In some embodiments, the subject is directed to a therapy that involves an anti-TIM-3 antibody, but excludes the use of a drug that targets PD-1 or PD-L1.

Various embodiments provide selecting a subject with tumors indicated to be unresponsive or refractory to a drug that targets PD-1 (as described in the prognosis methods above), and directing the subject to receive (or administering to the subject) an effective amount of a drug that targets TIM-3.

### System for Complex Spatial Analysis of Images

Systems are provided in at least one embodiment which relates to an image analysis system for performing any of the image analysis methods described above for tumor cluster identification, constructing a centroid, and/or calculating centroid distance.

In various embodiments, the system is configured for receiving at least one digital image of a tissue sample, typically based on imaging mass cytometry; analyzing the at least one received image for identifying immune cells and tumor cells in the image; identifying phenotypic clusters; constructing a centroid for each phenotypic cluster; for each identified phenotypic cluster, determining the distance of the cluster centroid to the nearest index cell (e.g., immune cell, or CD8+ T cells, or CD4+ T cells, or PD-1+ immune cells); computing a proximity profile of the tumor clusters as a function of the determined distance; classifying or zoning the identified tumor cells into tumor cells at an immune desert (with substantially no interaction with immune cells), dispersed tumor cells in immune activity zone (having relatively high interaction between tumor cells and nearby immune cells), or tumor cells at the interface with suppressive immune function; and storing the classification/zoning result on a storage medium and/or displaying the classification/zoning result on a display device.

### EXAMPLES

The following examples are provided to better illustrate the claimed invention and are not to be interpreted as limiting the scope of the invention.

### Example 1

The experience with PD1-PDL1 indicates that it may be relevant to understand which specific cell subsets in DLBCL express PD-L1 and whether other immunoregulatory proteins might be co-expressed on these PD-L1-positive cells that might alter the cell functions, making them non-responsive to PD-1/PD-L1 inhibitors yet possibly opening doors for other TO targets under investigation such as CCR4 and TIM-3 inhibitors.

CCR4 is a chemokine receptor for thymus and activation-regulated chemokine (TARC/CCL17) and macrophage-derived chemokine (MDC/CCL22), which is preferentially expressed on TH2 and T_{REG} cells. CCR4 is expressed in over 80% of adult T-cell leukemia/lymphoma (ATLL), and its expression on the tumor cells has been shown to correlate with unfavorable clinical outcome. Clinical trials with Mogamulizumab, a monoclonal antibody directed against CCR4, have shown tolerability and efficacy in R/R aggressive T-cell lymphoma. However, little is known about CCR4 in DLBCL.

T cell immunoglobulin and mucin domain-3 (TIM-3) is an immune-suppressive protein shown to enhance tolerance and inhibit TH1-mediated anti-tumor immune response when binding with its ligand galectin-9. TIM-3 has been highlighted as a prognostic marker and a promising target for immunotherapy in solid tumors. A number of ongoing phase-1 clinical trials investigating the safety of anti-TIM-3 as monotherapy or in combination with PD-1/PD-L1 inhibitors have shown promising safety profiles in patients with advanced solid cancers and R/R lymphomas.

DLBCL's immunophenotypic heterogeneity is driven in part by its TME that is composed with diverse cell types interacting with each other at the cellular level, making single-cell spatial interrogation crucial in unmasking the cellular and protein crosstalk in the TME. Other similar studies in this area have been limited by technical challenges—the conventional highly multiplexed single-cell techniques such as CyTOF or single-cell-RNAseq require tissue disruptions that lose all spatial information, while those that do retain tissue architecture can only examine a handful of markers simultaneously.

In the present study, we characterized the various components of DLBCL's TME, including their types, frequency, complexity and spatial interaction using imaging mass cytometry (IMC) that allows simultaneous detection of up to 34 markers and enables high-dimensional, single-cell analysis with spatial information of the different cell types at sub-cellular resolution. Using a panel of 32 antibodies, IMC was performed on tissue micro-array (TMA) slides from a retrospective cohort of 33 DLBCL patients treated with R-CHOP (rituximab plus cyclophosphamide, doxorubicin, vincristine, and prednisone, which is a standard-of-care treatment for DLBCL patients). In this study, we characterized the heterogeneity of immune infiltration and immune subset distribution in the TME across cases. Using multiplex imaging we identified combinations of biomarkers, such as PD-L1/TIM-3/CCR4, on tumor cells that predicted worse clinical outcomes, indicating that DLBCL patients could potentially benefit from TIM-3 and CCR4 inhibitors currently under clinical investigation. Furthermore, we developed a novel method of analyzing the complex interactions among CD8 T cells, tumor and other immune cells in the TME. Our spatial analyses identified distinct clusters, or “neighborhoods”, within the TME that correlated with treatment response/resistance. This spatial clustering method yielded insights into the functional statuses of immune cells in the TME. Finally, we were able to incorporate spatial information into our analysis to improve the predictive power of our biomarkers.

Taken together, this study highlights the power of multiplex phenotypic and spatial characterization of the TME for understanding tumor biology, and as a source of biomarkers to guide the development of new immune-oncology therapies for DLBCL.

Results. IMC Analysis of DLBCL's TME Identifies Marked Heterogeneity of Immune Infiltration and Cellular Subtypes.

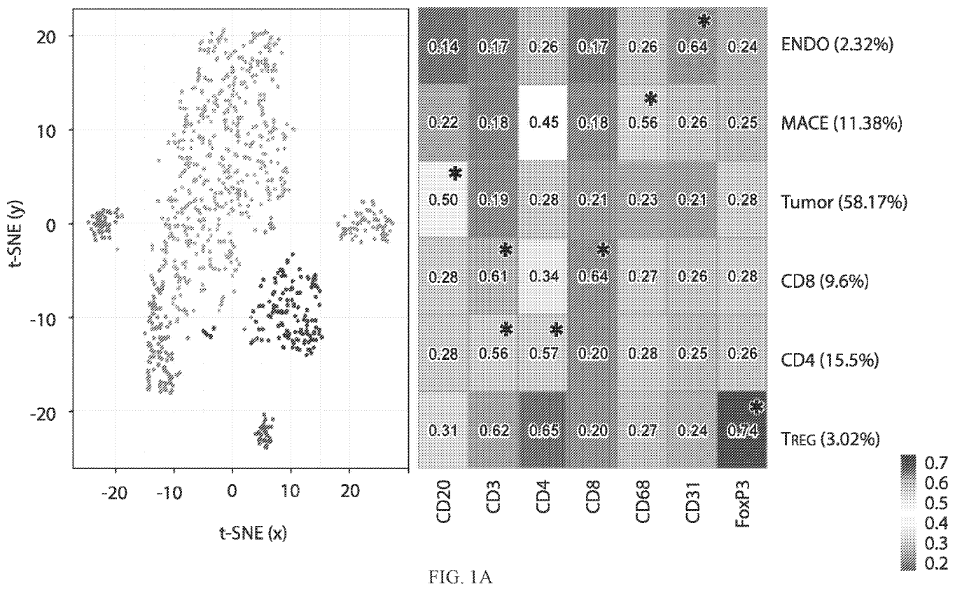

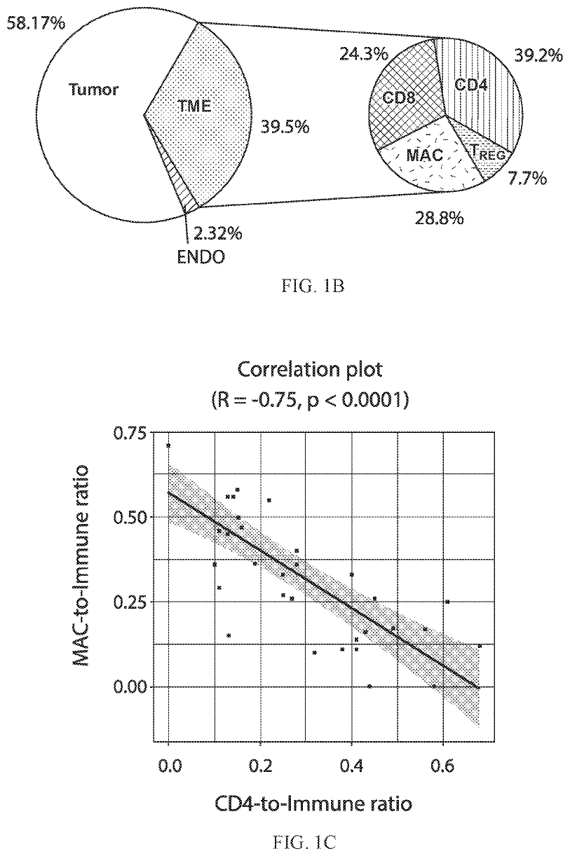

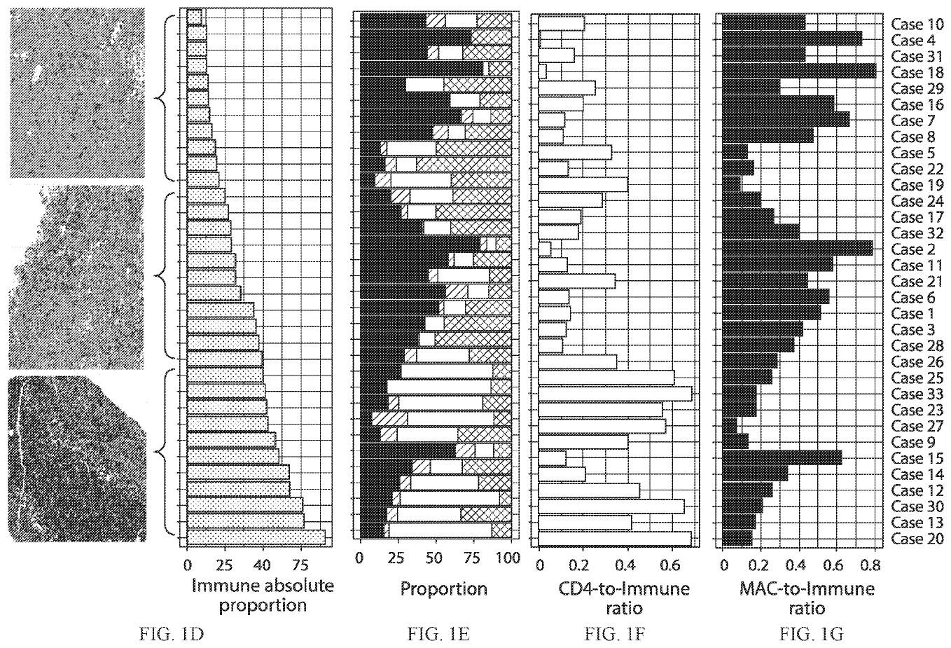

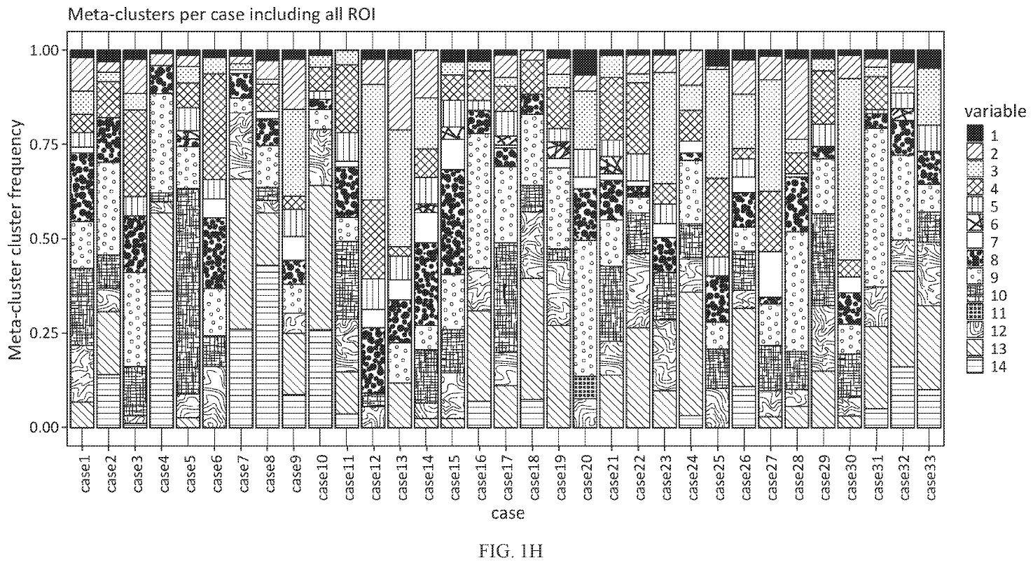

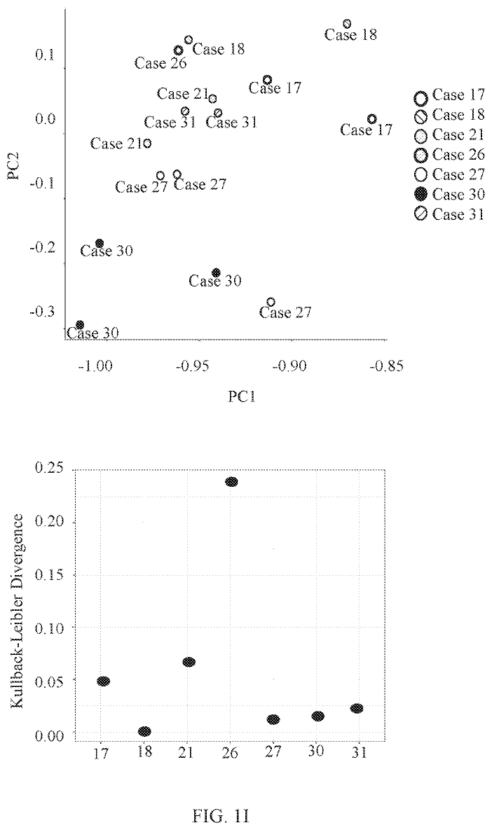

We designed an IMC panel to broadly cover T cells, B cells, Macrophages and structural markers (Table 2) and used it on 41 representative tumor samples to profile the TME of 33 de novo DLBCL patients (“33 cases”), selected from a previously described cohort (described in Am J Clin Pathol 2014; 141:593-604) based on adequacy of residual tissue for analysis: based on H&E staining images of sections from each patient were included in the analysis; PCA (principle component analysis) plot shows that the duplicates cluster together, indicating that small sample areas may be adequate to understand the complexity of tumor immune responses; the PCA of the phenotype proportions of replicates was plotted, and the Kullback-Leibler (KL) divergence scores were computed where the x-axis denotes the case number (FIG. 1I). Case 26 replicate ROIs had the highest entropy indicating that in this case the duplicates are heterogeneous. In this study we averaged the replicates at the case level using a mixed-effects linear model.

Our panel of 32 antibodies quantified exhaustion (TIM-3, LAG-3, PD-1) and proliferation (Ki67) states and included markers of immune and endothelial cell lineages. These findings were correlated with clinical features such as cell of origin (COO) by IHC HANS criteria, presence of mutations, and responses to chemotherapy (Table 1). A tonsil section was stained with the 32-antibody panel at the same titers used to stain the DLBCL TMAs; PD-L1 positivity was validated using HDLM-2 cell line; and PC-3 cell line was used as PD-L1 negative control; and the 32-antibody panel included those targeting CD20, CD3, CD4, CD8, CD45RA, CD45RO, Ki-67, PD-1, TIM-3, LAG-3, OX40, VISTA, Tbet, FoxP3, Granzyme-B, CCR4, CXCR3, ICOS, BCL2, BCL6, C-MYC, CD68, CD206, Vimentin, CD31, CD34, HLA-DR, pSTAT3, PD-L2, and Ephrin-B2. In the cohort, 22 (67%) of the patients achieved a sustained complete response (CR) to rituximab based chemoimmunotherapy, while 7 (21%) were primary refractory or relapsed within one year. Four patients did not receive chemotherapy and were excluded from response to therapy analysis.

TABLE 1
Clinico-pathological characteristics of the cohort.

| Parameters | n (%) |
|---|---|
| Age, mean ± SD | 49 ± 15 |
| (range) | (20-82) |
| Sex | |
| F | 9 (27%) |
| M | 24 (73%) |
| Ethnicity | |
| Black | 1 (3%) |
| Asian | 5 (15%) |
| Caucasian | 2 (6%) |
| Hispanic | 25 (76%) |
| LDH | |
| Not elevated | 16 (49%) |
| Elevated | 17 (51%) |
| IPI score | |
| 0-2 | 23 (70%) |
| 3-5 | 10 (30%) |
| EBER | |
| Positive | 6 (18%) |
| Negative | 27 (82%) |
| Hans COO | |
| GCB | 17 (51%) |
| Non-GCB | 16 (49%) |
| Mutation status | 22 (67%) |
| TP53 | 8 (24%) |
| BCL2 | 3 (9%) |
| BCL6 | 2 (6%) |
| CD79b | 6 (18%) |
| SGK1 | 1 (3%) |
| MYD88.L265P | 2 (6%) |
| NOTCH1 | 3 (9%) |
| NOTCH2 | 3 (9%) |
| EZH2 | 4 (12%) |
| 9p24 | 8 (24%) |
| chromosomal gain | |
| Stage | |
| I/II | 15 (45%) |
| III/IV | 18 (55%) |
| III/IV | 18 (55%) |
| Response status | |
| CR | 22 (67%) |
| REF | 7 (21%) |
| Unknown | 4 (12%) |
| Treatment | |
| Chemo- | 29 (88%) |
| immunotherapy | |
| Not done/LTF | 4 (12%) |

COO: cell of origin;
GCB: Germinal Center B Cell;
Non-GCB: Non-Germinal Center B Cell;
CR: Complete Response;
REF: Refractory to treatment;
LTF: Lost to follow up;
EBER: Epstein-Barr virus encocled small RNAs;
LDH: Lactate dehydrogenase serum levels. Double-hit tumors were excluded from this data set.

IMC produces images similar to immunohistochemistry or immunofluorescence with the added advantage of increased multiplex staining. The images were segmented using pixel classification training into single cells, which yielded on average 16,889 cells per TMA (tissue microarray) core ablated, i.e., on average 16,889 cells per region of interest (ROI). In one example, H&E images of representative cases 16 and 33, as well as respective IMC images of cases 16 and 33 showing corresponding/matching spatial distributions of tumor cells, CD4 T cells, CD8 T cells, T_{REG}, macrophages and endothelial cells, which further highlighted the accuracy of segmentation.

Cellular expression of cell lineage, inducible states, and spatial features were hierarchically clustered with Phenograph to identify major phenotype clusters such as endothelial, regulatory (T_{REG}), CD8 T cells, CD4 T cells, macrophages, and B cell tumors (FIG. 1A).

t-Stochastic neighborhood embedding (tSNE) graph of the full cohort identified major lineage normalized expression marker intensity (cell components at the cohort level), which indicates cluster homogeneity for primary lineage markers at the cohort level. Hierarchical meta-clustering across ROI first identified 14 meta-clusters which were well distributed across cases (FIG. 1H showing the case relative proportion of the meta-clusters). We observed overall well distributed cluster, without any case specific meta-cluster. The clustering was performed across each ROI, but the centroid (median) expression was used to pool clusters into the meta-cluster level. The initial meta-clusters were constructed and annotated based on lineage marker expression which identified the broadest categories of the TME phenotypes in DLBCL. We also performed quality control analysis to ensure that cluster annotations were appropriately defined. Specifically, we examined each major cell component (CD4, CD8, T_{REG}, B-Cell tumor, endothelial) using sub-clustering re-assignment to ensure that all sub-clusters held homogeneous expression of the lineage expression. We re-assigned any sub-cluster that did not fit the corresponding major component being analyzed. Each major cell component was sub-clustered to optimize homogeneity of lineage specific markers which corresponded with the canonical phenotype of each primary phenotype. CD4 (denoted Th) had primarily uniform expression of CD3 and CD4, with the exception of sub-clusters Th_9 (re-assigned to CD8), Th_12, and Th_21 (re-assigned to T_{REG}). Although Th_8 had dim CD4 expression, it did not have over-expression of an alternative lineage marker and was left in the CD4 component. We further performed quality control analysis on the tumor clusters by sub-clustering each major cell component in anticipation of uniform expression of the major component marker within each sub-cluster. A uniform expression of CD20 was identified in the sub-clusters within the “tumor” component. For the sub-clusters that had dim CD20, they were re-assigned to an appropriate alternative major cell component. The tumor sub-clusters allowed for the reassignment of ‘tumor’ major cell components with dim CD20, into other major components. The T_{REG} and CD8 primary phenotypes did not identify any re-assignments because they had uniform expression of their canonical protein (FOXP3 and CD8, respectively). Similarly macrophages and endothelial components did not require reassignments.

The major cell components across the 33 cases included 58.2% malignant B-cells, 39.5% TME, and 2.32% endothelial cells (FIG. 1B). Within the TME, we identified 7.70% T_{REG}, 24.3% CD8 T cells, 28.8% macrophages and 39.2% CD4 T cells. Macrophage proportions were 3.96% (p=0.069) higher in non-GCB compared to GCB cases. In tumors with low immune infiltration (FIGS. 1D, 1E), macrophages were the predominant immune cell type, whereas in cases with greater immune infiltrate CD4+ cells predominated (FIGS. 1F and 1G)—the 33 cases had different immune absolute proportions from 9.42% to 90.14%. This resulted in a significant negative association between the macrophage and CD4+ cell proportions (Pearson's R=−0.75, p<0.001) across the 33 cases (FIG. 1C) as was similarly reported for solid tumors.

### Association Between Cell of Origin and Abundance of Cell Phenotypes in DLBCL TME.

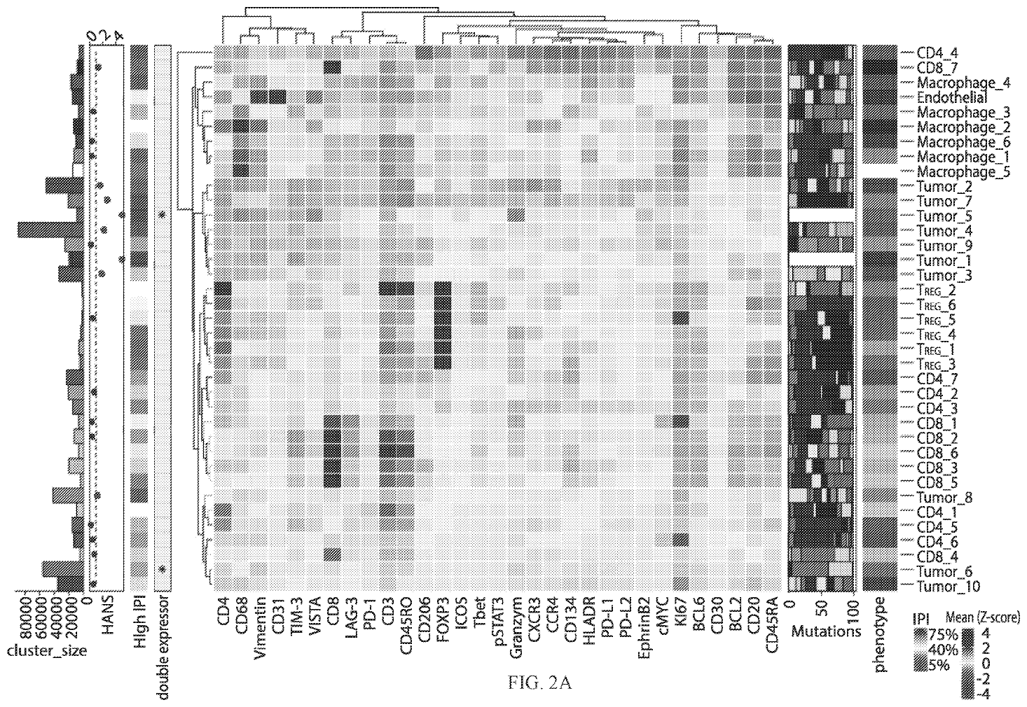

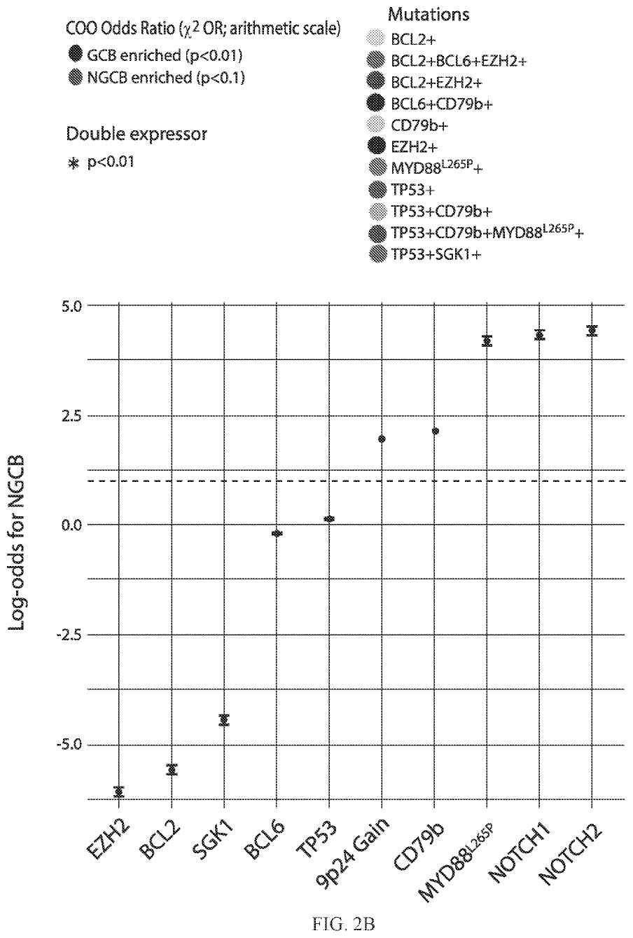

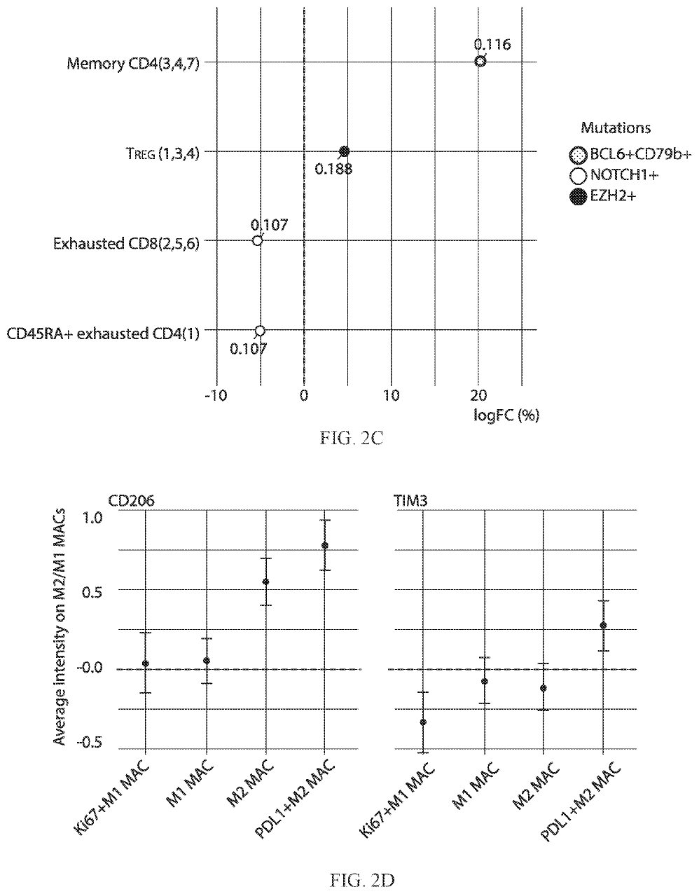

We next performed sub-classification of each major cell component by including inducible markers and morphological features, which identified the functional states of tumor and immune sub-phenotypes in the TME (FIG. 2A). We then associated these sub-clusters with COO, international prognostic index (IPI), and tumor mutations to gain insight into the biological significance of the observed tumor and immune heterogeneity.

The CD4 sub-cluster 1 (TME prevalence=3.7%), or CD4_1, had the strongest association with GCB (Chi-square OR_{GCB}=1.29, median-unbiased estimation p (mid-p)=0.013), and had the highest PD-1 mean normalized intensity (PD-1 estimate=0.56, 95% CI: (0.46, 0.66), p<0.001). To calculate standardized expression Z-score of each marker across ROI, the min/max normalized scores of all cells for each marker were standardized to a Z-distribution across ROIs (i.e., ‘mean normalized intensity’), and the markers included BCL2, BCL6, CD20, CD206, CD3, CD31, CD4, CD45RA, CD45RO, CD68, CD8, FOXP3, HLADR, CCR4, CXCR3, Granzym, ICOS, Ki67, Lag3, PD1, PDL1, Tim3, Vista, Vimentin, EphrinB2, cMYC, CD134, PDL2, and pSTAT3.

CD4_1 was labeled as the CD45RA⁺ exhausted phenotype because of its high levels of CD45RA, LAG-3 (LAG-3 estimate=0.41, 95% CI: (0.26, 0.55), p<0.001), and TIM-3 (TIM-3 estimate=0.55, 95% CI: (0.38, 0.71), p<0.001). Whereas CD4_5 (TME prevalence=6.03%) was significantly associated with non-GCB (OR_{GCB}=0.24, mid-p<0.001) and was defined as the CD45RA⁻ exhausted phenotype due to its low CD45RA intensity (CD45RA estimate=−0.97, 95% CI: (−1.17, −0.76), p<0.001), and increased expression of LAG-3 (LAG-3 estimate=0.59, 95% CI: (0.45, 0.73), p<0.001) and TIM-3 (TIM-3 estimate=0.54, 95% CI: (0.37, 0.79), p<0.001). CD4_3, CD4_4, CD4_7 sub-clusters had similar phenotype and were combined into a memory CD4 (TME prevalence=16.3%) group. This group had low levels of exhaustion markers, and was present in both COO types at similar levels. Across patients, CD4 sub-clusters had significant expression variability of CXCR3, CCR4, PD-1, PD-L1, ICOS, Ki67, and VISTA.

The M1 macrophage (Macrophage 3, Macrophage 4) phenotype family was not associated with either COO type, whereas the M2 (Macrophage 1, Macrophage 5, and Macrophage 6) family was enriched with NGCB (OR_{GCB}=0.39, mid-p<0.001). The M2 macrophages (TME prevalence=19.6%) had significantly increased CD206 expression levels whereas M1 macrophages (TME prevalence=9.3%) did not (FIG. 2D). Macrophage_2 (TME prevalence=4.9%) has a similar profile to M2 macrophages, but also had high PD-L1 (PD-L1 estimate=0.60, 95% CI: (0.5, 0.71), p<0.001), and TIM-3− moderate (TIM-3 estimate=0.27, 95% CI: (0.12, 0.43), p=0.012) was labeled as the PD-L1⁺M2 macrophage. The PD-L1⁺M2 immunosuppressive phenotype was weakly associated with GCB (OR_{GCB}=1.06 mid-p=0.44).

The T_{REG}_5 cluster (TME prevalence=0.71%) was labeled as the Ki67⁺ T_{REG} phenotype and had an activated proliferative phenotype with high Ki67 (Ki67 estimate=1.78, 95% CI: (1.63, 1.92), p<0.001), and was associated with non-GCB (OR_{GCB}=0.47, mid-p=2.1e-04). T_{REG}_2 and T_{REG}_6 were grouped together as “highly suppressive T_{REG}” (TME prevalence=1.2%) because of high expression of CCR4 (CCR4 estimate=0.52, 95% CI: (0.37, 0.66), p<0.001) and ICOS (ICOS estimate=0.20, 95% CI: (0.08, 0.32), p=0.02). Highly suppressive T_{REG} were marginally represented in NGCB subtypes (OR_{GCB}=0.76, mid-p=0.08). The remaining T_{REG} sub-clusters were defined broadly as “T_{REG}” (TME prevalence=5.71%) which included cluster such as T_{REG}_1, T_{REG}_3, and T_{REG}_4, which were not associated with either COO subtype.

The CD8 sub-clusters had significant variability of TIM-3, PD-1, and LAG-3. The Ki67⁺CD8 phenotype (TME prevalence=4.4%) included CD8_1 and CD8_4 and exhibited an activated phenotype (Ki67 estimate=1.35, 95% CI: (1.23, 1.49), p<0.001), high PD-1 expression (PD-1 estimate=0.34, 95% CI: (0.25, 0.44), p<0.001) and was highly associated with non-GCB (OR_{GCB}=0.48, p<0.001). Although this population was PD-1⁺, it had low expression of other exhaustion markers. In contrast, CD8_6 (TME prevalence=2.4%) was non-proliferative and had high TIM-3 (TIM-3 estimate=0.86, 95% CI: (0.69, 1.03), p<0.001), high PD-1 (PD-1 estimate=0.44, 95% CI: (0.34, 0.54), p<0.001), and high LAG-3 (LAG-3 estimate=1.1, 95% CI: (0.96, 1.25), p<0.001), and was classified into an exhausted phenotype. Combined with other sub-clusters of similar phenotype, the exhausted CD8 phenotype family (CD8_2, CD8_5, CD_6) represented 8.88% of the TME and was significantly enriched in non-GCB (OR_{GCB}=0.59, mid-p<0.001). Both activated and exhausted populations expressed PD-1, demonstrating the limitations in using PD-1 as a single marker for exhausted T cells in tissue sections. Interestingly both CD8 exhausted and proliferative phenotypes were more prevalent in NGCB type tumors, which typically have a poorer prognosis.

### Association Between Genetic Mutations and Abundance of Cell Phenotypes in DLBCL TME.

Twenty-two of our patients were subjected to mutation screening using a non-Hodgkin lymphoma-specific gene panel. We confirmed previously reported associations between COO, and specific mutations (FIG. 2B). EZH2, BCL2, and SGK1 mutations were associated with GCB, whereas CD79B, NOTCH1/2, and MYD88^{L265P} were associated with non-GCB.

We then established the association between mutation and cluster proportions identified by IMC. Using the case relative proportions, the memory CD4 phenotype was associated with co-mutations BCL6⁺CD79b⁺ (log FC=20.2%, Benjamin-Hochberg (BH) q-value=0.116) indicating that subjects with this co-mutation tended to have increased proportions of this memory CD4 phenotype (FIG. 2C). CD45RA⁺ exhausted CD4 phenotype (CD4_1) was enriched in GCB subjects, but this phenotype had decreased proportions in subjects with NOTCH1 mutation (CD4_1: log FC=−5.05%, BH q-value=0.106). Similarly, the exhausted CD8 phenotype had decreased abundance in patients with NOTCH1 mutations (log FC=−5.4%, BH q-value=0.10). The cell-of-origin classification in DLBCL identified heterogeneity from a landscape mutational profile, of which NOTCH1 and co-mutations of BCL6/CD79b also influence the immune microenvironment.

Each patient's chromosomal abnormalities were evaluated using comparative genomic hybridization (CGH), and we observed that 9p24 copy gains or losses were not associated with tumor abundances (p=0.58, ANOVA) and marginally associated With tumor PD-L1 expression (p=0.21, ANOVA), although this is likely due to the limited sample size as 9p24 copy gains have been previously associated with PD-L1 expression in this cohort. Although our IMC panel was primary focused on the TME and did not include the markers needed for COO classification by HANS, the immuno-morphological tumor clusters identified by IMC aligned well with mutational clusters and COO classification. Follow up work using a tumor focused panel of markers would likely identify additional tumor heterogeneity.

### Association Between Chemo-Responsiveness, Tumor Phenotypes, and TME.

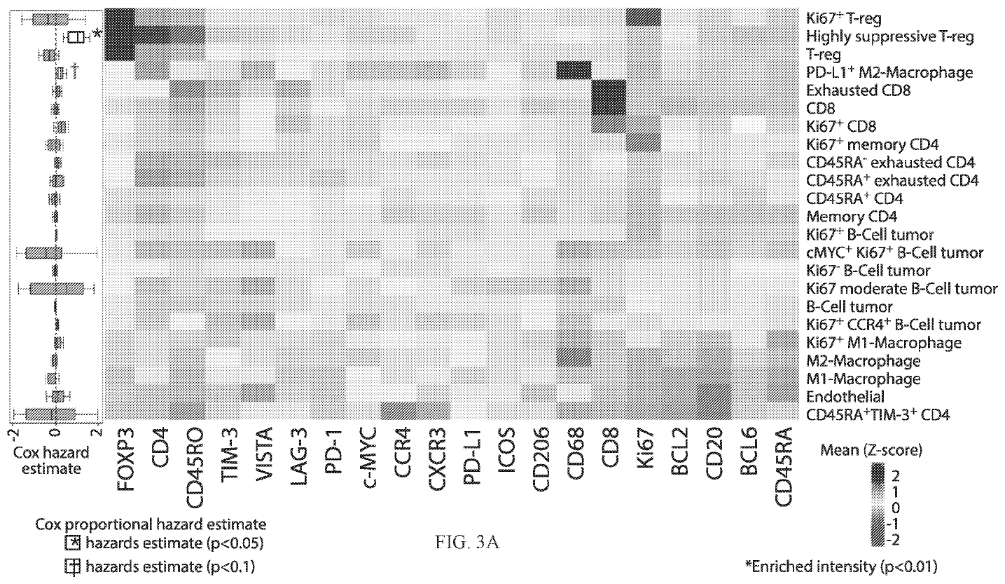

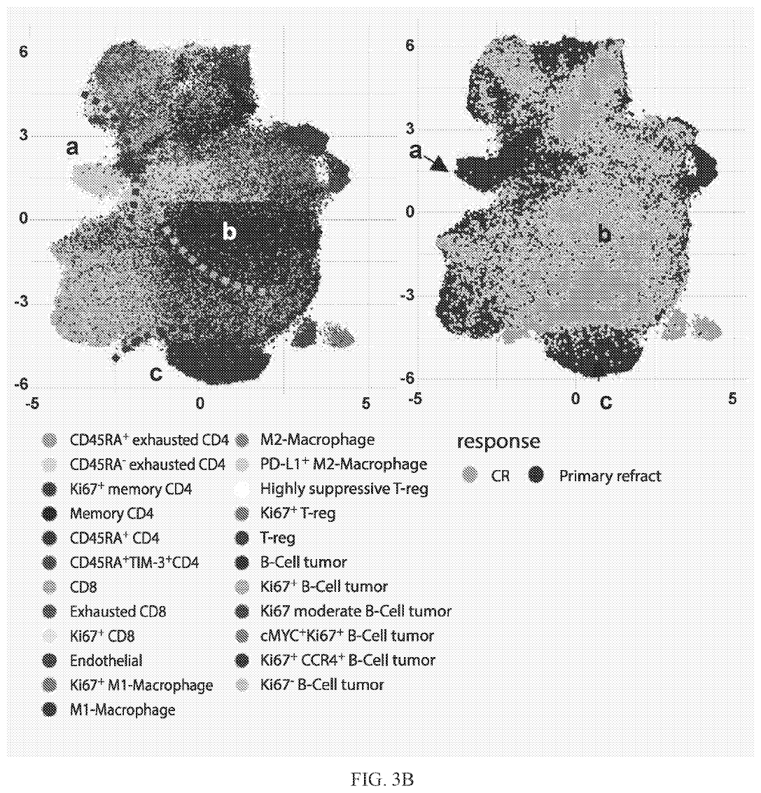

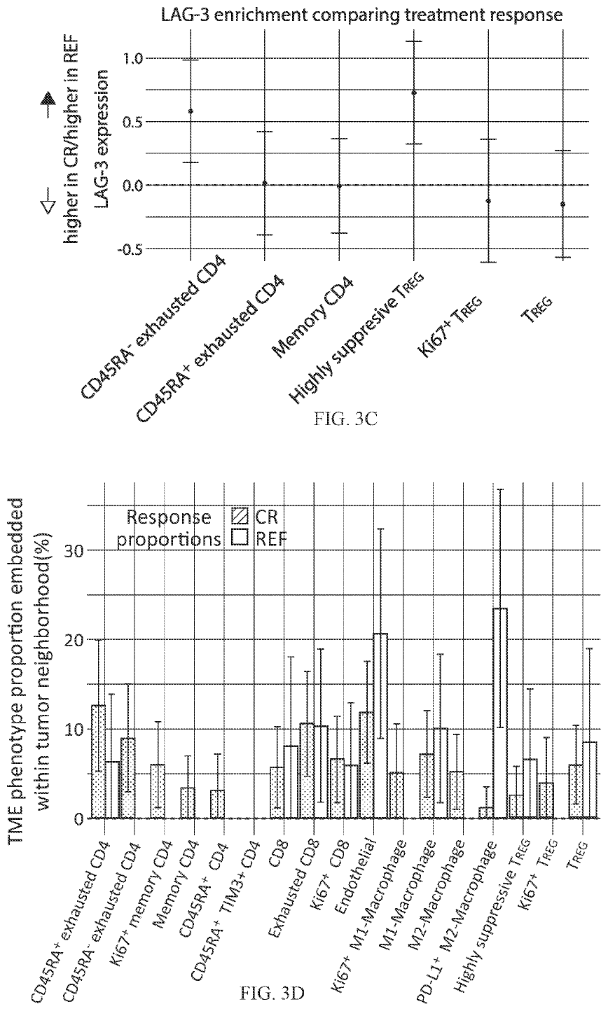

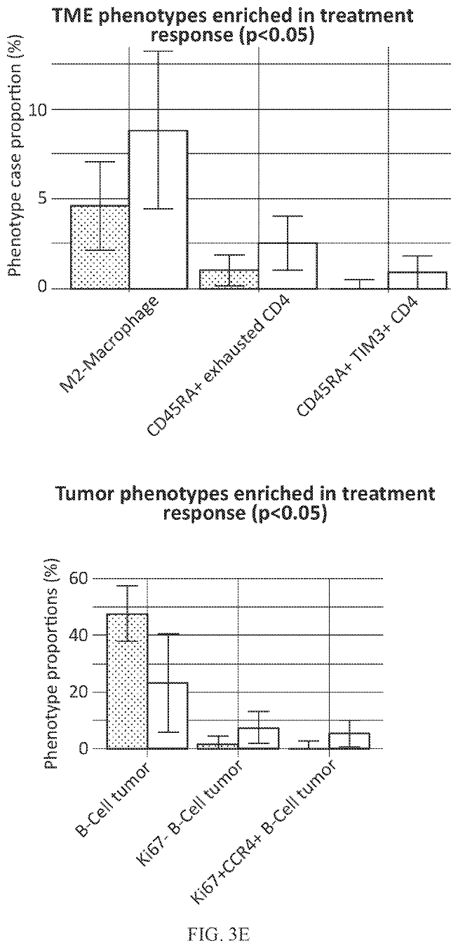

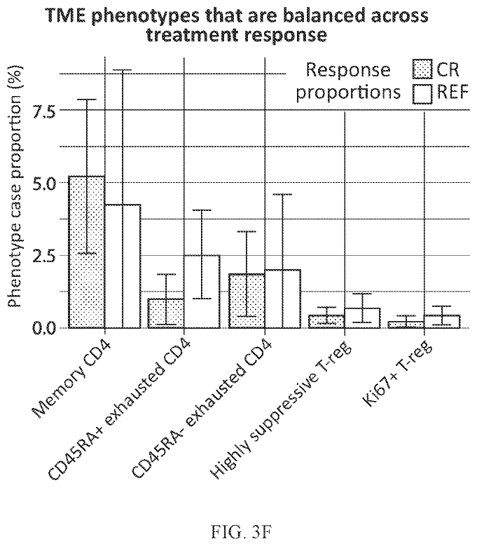

In order to understand how tumor and TME interactions contribute to chemo-responsiveness, we examined the abundance and spatial interactions of tumor and immune cell clusters in relation to clinical response to chemotherapy. To examine global differences associated with treatment response, we computed univariate Cox proportional hazard estimates using the abundances of each cell type and found a highly suppressive T_{REG} population to be the most significantly associated with being refractory to chemotherapy (Hazard ratio=2.44, 95% CI: (1.17, 5.11), p=0.017) (FIG. 3A). PD-L1⁺M2 macrophage relative abundance was marginally associated with poor response to chemotherapy (Hazard ratio=1.30, 95% CI: (0.99, 1.70), p=0.059).

We then performed Uniform Manifold Approximation and Projection (UMAP) of all samples to visualize distinct clusters of tumor and immune cells (FIGS. 3B, 3E). In particular, we were interested in understanding heterogeneity of PD-L1 expression on tumor cells as this has been previous associated with poor prognosis after chemotherapy. The largest cluster (67.9%) of tumor cell was labeled “B cell tumor” (region b) and was enriched in complete responders (“CR”) and did not express PD-L1. Tumor regions c and a were enriched in refractory patients. Cluster a was identified as Ki67⁻ tumor and had high PD-L1 expression (PD-L1 estimate=+0.33, 95% CI: (0.22, 0.43), p<0.001) while cluster c was identified as Ki67⁺CCR4⁺ tumor and had low PD-L1 expression (PD-L1 estimate=−0.21, 95% CI: (−0.32, −0.09), p<0.001). These data confirm that PD-L1 expression was associated with refractoriness to chemotherapy, but that its expression was limited to a certain sub-population of non-proliferating tumor cells.

This finding indicates the interesting possibility that chemoresistance is mediated by a non-proliferating, immune evading subpopulation of tumor cells.

We then examined the TME for markers that associated with chemotherapy response. Comparing refractory (REF) to complete responder average intensity, we observed that LAG-3 was increased in REF subjects, specifically in the CD45RA⁻ CD4 exhausted phenotype (LAG-3 difference estimate=0.58, 95% CI: (0.18, 0.98), p=0.10) and significantly increased in highly suppressive T_{REG} (LAG-3 difference estimate=0.72, 95% CI: (0.32, 1.13), p=9.1e-03) (FIG. 3C). Additionally, both phenotype case proportions did not differ between REF to CR, although LAG-3 expression was enriched in REF subjects (FIGS. 3C and 3F) and is consistent with a recent report on the LAG-3 expression in DLBCL.

Immune cells in the TME can mediate chemoresistance through direct interaction with tumor cells or indirectly through interactions with other immune cells. Therefore, we sought to characterize the hyper-local microenvironment within 2-3 cell diameters (15 μm) of REF enriched tumor cells (FIG. 3D). Comparing the tumor neighborhoods across treatment response, the REF tumor neighborhoods had significantly increased attractions with PD-L1⁺M2 macrophages (p=0.04831, t-test); however highly suppressive T_{REG}, which were highly associated with REF at the case level, showed only non-statistically significant enrichment in the hyper-local TME (p=0.74, t-test). These indicate the PD-L1⁺M2 macrophages mediate chemoresistance through direct interactions with tumor cells, while highly immunosuppressive T_{REG} exert their effects primarily through interactions with other immune cells. In contrast, the hyper-local tumor neighborhoods associated with CR had uniformly increased CD4 phenotypes within the tumor environment compared to REF (p<0.05, t-test). These results provide evidence that immune cells in the TME can have a direct effect on modulating chemoresistance of malignant B cells.

### Cross-Cohort Analysis Shows Differences in Proportion and Functional States of Immune Cell Subsets Between DLBCL and Hodgkin Lymphoma.

For patients who fail chemotherapy, treatment with antibodies against the PD-1/PDL-1 checkpoint have elicited clinical response rates close to 90% in patients with HL, while responses in patients with DLBCL are less than 10%. Therefore, we hypothesized that comparing the TME between HL and DLBCL might reveal the basis for the difference in clinical responses to checkpoint inhibitors. We previously reported our analysis of the TME in 22 cases of HL using a 36 marker IMC panel, of which 21 markers overlapped with the IMC DLBCL panel. Specifically, the Hodgkin's lymphoma meta-clustering analysis and annotation of major cell components was performed; and each meta-cluster expression was identified and annotated into major cell lineages—canonical phenotypes include B cell, CD4+ cell, CD8+ cell, dendritic cell (DC), endothelial, granulocytic myeloid-derived suppressor cell (MDSC), Hodgkin and Reed/Sternberg (HRS) cell, macrophage, and Treg, whereas lineage expression markers include CD11b, CD11c, CD14, CD15, CD16, CD206, CD20, CD30, CD34, CD3, CD45RA, CD45RO, CD4, CD68, CD8a, FOXP3, and EphrinB2. We integrated the TME analysis from both diseases after sub-setting each experiment to include only CD4, CD8, MAC, and T_{REG}, and identified the joint TME phenotypes (FIG. 4A).

After integrating both experiments, we carefully tested for the presence of batch effects (FIGS. 4E, 4F). By visual inspection of principal component analysis (FIGS. 4B, 4G), the TME expression levels from both experiments were well inter-mixed. Specifically, the standardized and batch normalized expression (Z-score) across the TME in DLBCL and Hodgkin lymphoma were plotted for each ROI, including the expression of each of CD4, CD8a, CD68, FOXP3, CD206, and DNA1. By visual inspection, we observed similar dynamic ranges with moderate skewness in CD206 and CD68. The PCA analysis in FIG. 4B was a patient level average of average markers (TIM-3, CD4, CD68, CD8, FOXP3, PD-1, PD-L1, CD206); whereas a PCA and UMAP at the single cell level visually indicated that the batch correction had minimum cohort/experimental bias using the same markers. FIG. 4G shows the joint immune phenotype clusters of the DLBCL and HL TME. This was used in supervised annotation of FIG. 4A. Positive “+” calls used 0.49-0.5 cut-offs at the cluster level. The standardized expression (Z-score) across the TME in DLBCL and Hodgkin's lymphoma were plotted for each ROI, including expression of each of PDL1, PD1, TIM3, CCR4, CXCR3, ICOS, LAG3 (excluded) and Ki67, as well as cell area. By visual inspection, we observed dis-similar dynamic ranges of LAG-3, which indicated they had different experimental optimizations, hence LAG-3 was excluded. Ki67 was marginal and was used in the analysis. In both experiments the cell areas of TME were very similar indicating that the morphological features in both experiments were similar.

We quantitatively tested the level of inter-mixing between the two experiments using the K-nearest neighbor batch effect test and confirmed that the joint panel expression intensities on the TME were homogeneous in their expression (CD4 p=0.31, CD8 p=0.49, MAC p=0.43, T_{REG} p=0.08) (FIG. 4E, 4F).

Comparing the TME phenotypes identified in DLBCL to HL, macrophages were more abundant in DLBCL compared to HL (FIG. 4C), but their global PD-L1 expression was lower in DLBCL (log FC=−0.26, BH q-value=7.35e-14) (FIG. 4D). These results confirm the previously reported importance of PD-L1 on macrophages in HL. The relative proportion of TIM-3⁺PD-L1⁺ macrophage phenotypes were similar in both HL and DLBCL (Log-odds=−0.2, p=0.55), however TIM-3⁻PD-L1⁺ macrophages had the highest enrichment in DLBCL compared to HL (Log-odds=5.1, p<0.001). CD4 cells were more abundant in HL compared to DLBCL, but the subset of TIM-3⁺ T cells was increased in DLBCL (log FC=0.46, BH q-value=2.3e-08) (FIGS. 4C and 4D). This corresponded with the TIM-3⁻PD-1⁺CD4 T cells significantly enriched in HL compared to DLBCL (Log-odds=0.38, p<0.001).

CD8 T cells were found in equal proportion in DLBCL and HL, however CD8 T cells had higher PD-1 [log FC=0.35, BH q-value=8.9e-09] and TIM-3 [log FC=0.44, BH q-value=2.3e-08] expression in DLBCL. Further, the TIM-3⁺PD-1⁺CD8 phenotype was significantly enriched in DLBCL compared to HL (Log-odds=1.05, p<0.001), whereas TIM-3⁺PD-1⁻CD8 were significantly associated with HL (Log-odds=−1.7, p<0.001). Finally, T_{REG} cells were less abundant in DLBCL compared to HL (Bonferroni adjusted p-value=2.3e-05), but the proportion of the PD-1⁺ T_{REG} subset was increased (log FC=0.23, BH q-value=7.3e-05) while the PD-1⁻Ki67⁺ T_{REG} was significantly enriched in HL (Log-odds=−3.7, p<0.001) (FIG. 4A, 4D). The data indicates that PD-1 antagonists in DLBCL could re-activate immuno-regulatory functions of T_{REG} and support the use of alternative checkpoint inhibitors such as anti-TIM-3 antibodies in DLBCL. Finally, there results demonstrate that comparisons of TME between ICI responsive and non-responsive sub-types of lymphoma can indicate potential mechanisms of ICI sensitivity and resistance.

### Spatial Analyses Reveal Differences in Tumor Topology Between GCB and Non-GCB.

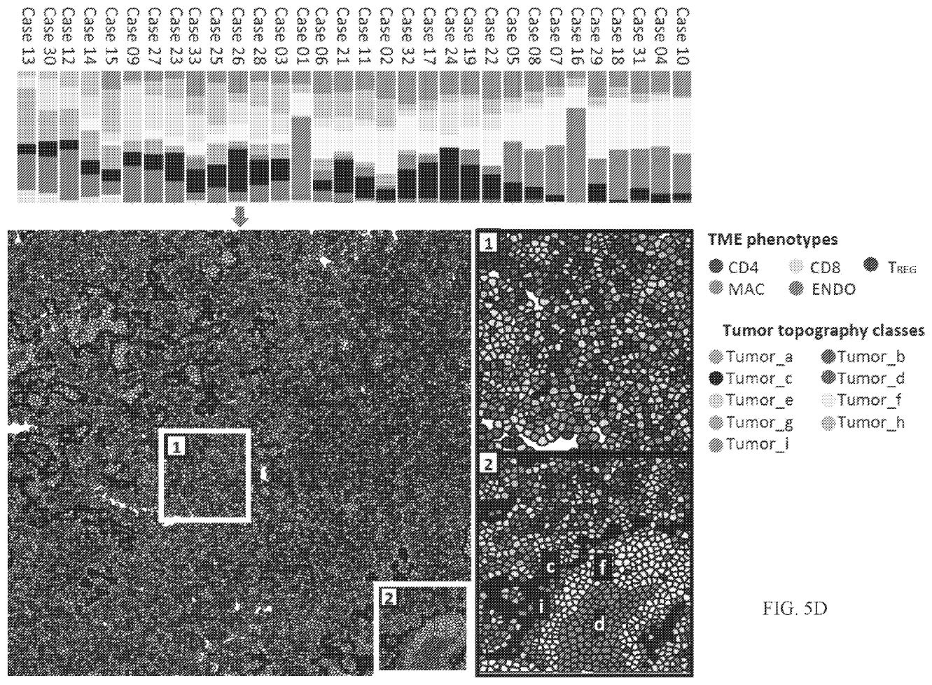

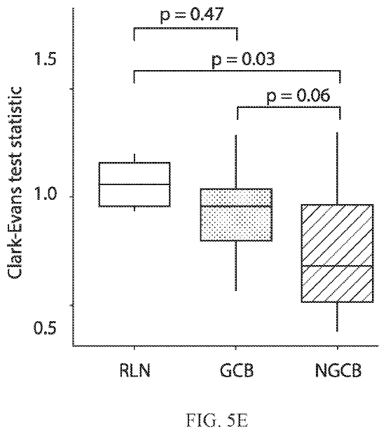

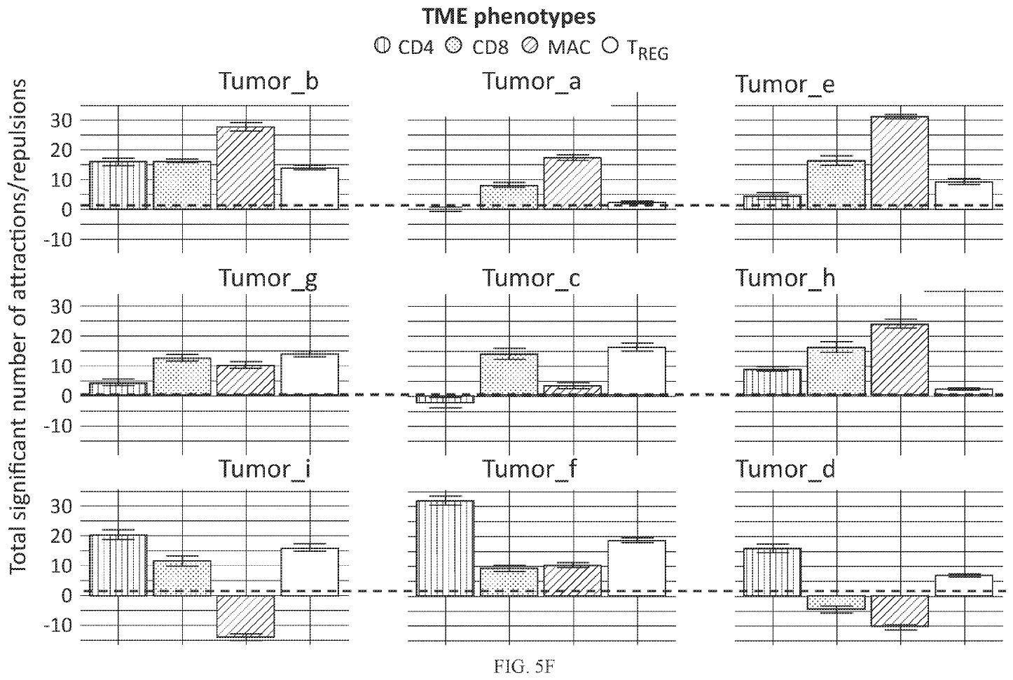

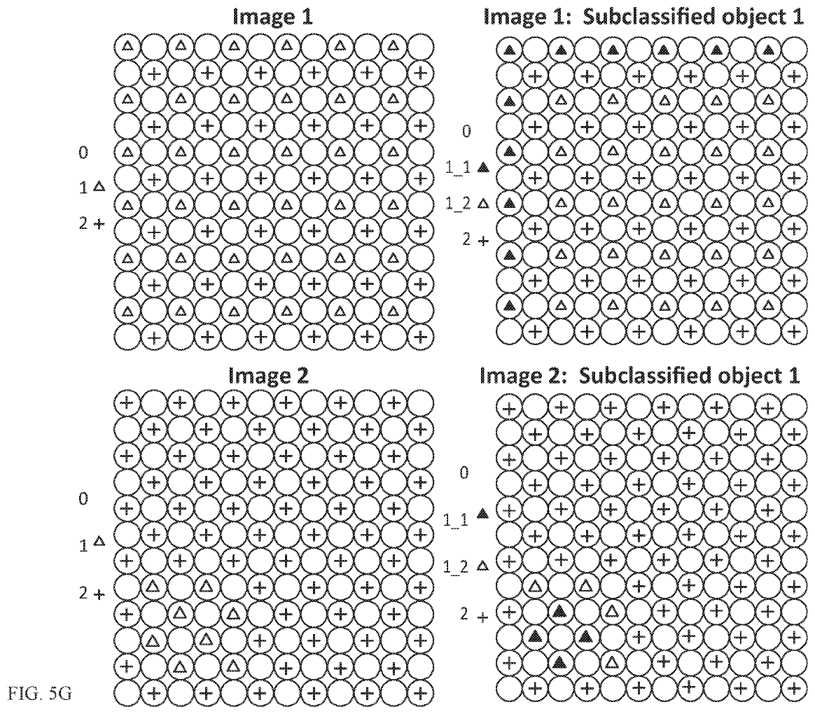

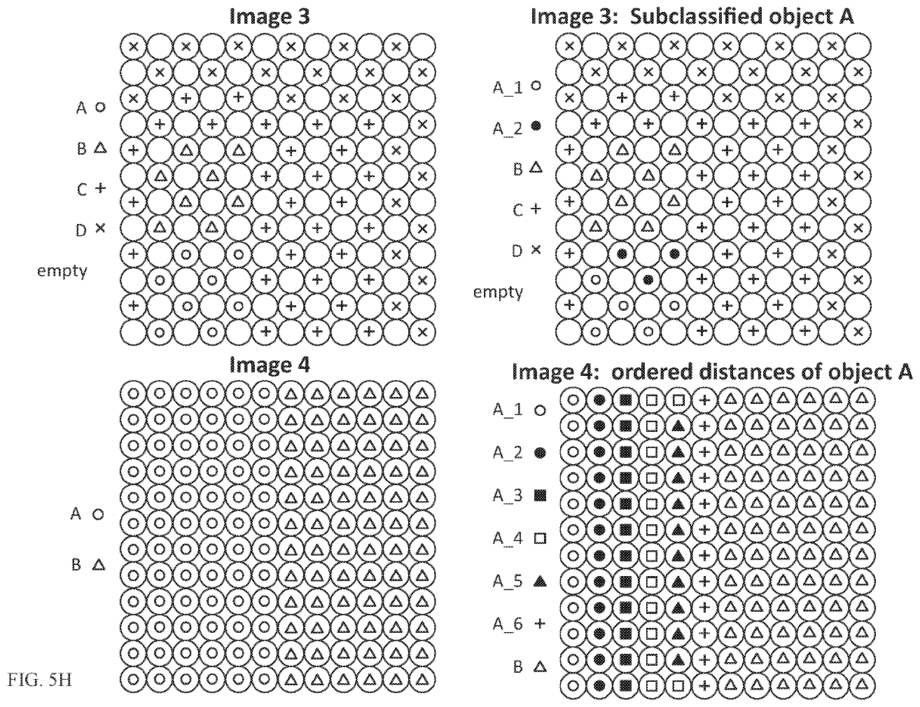

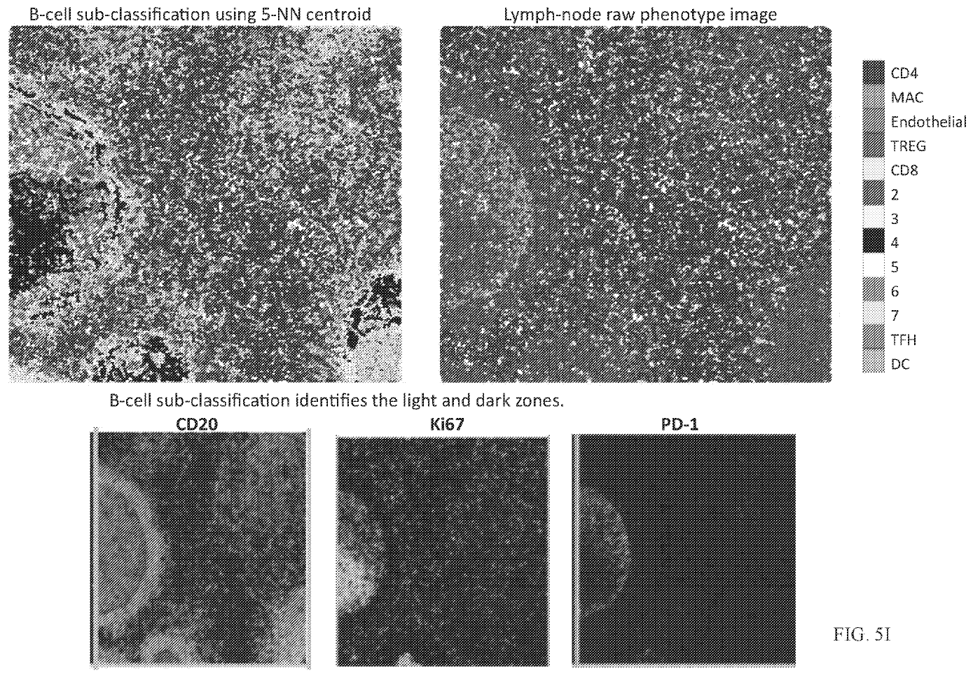

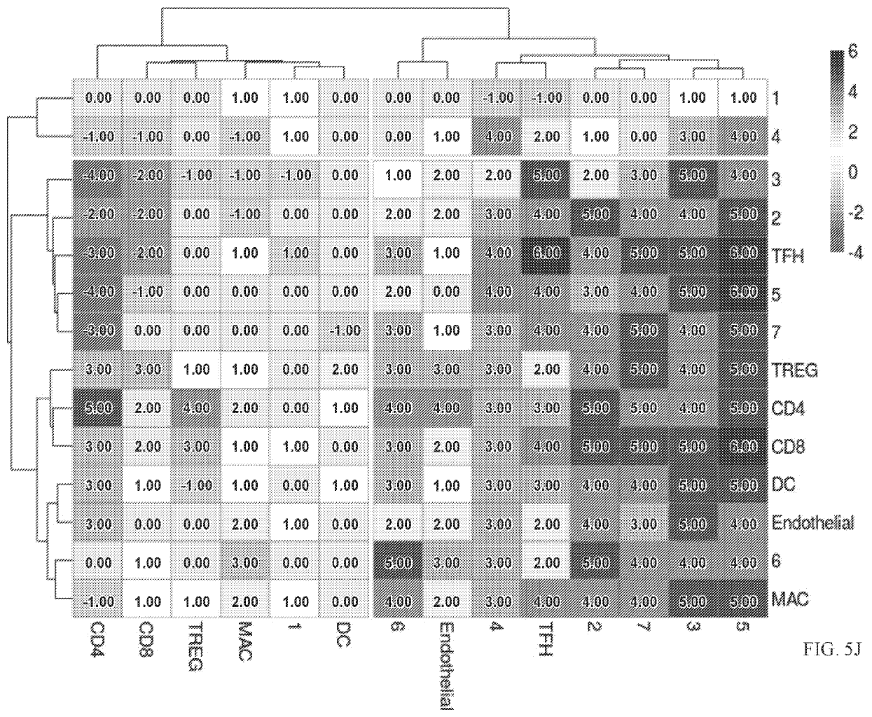

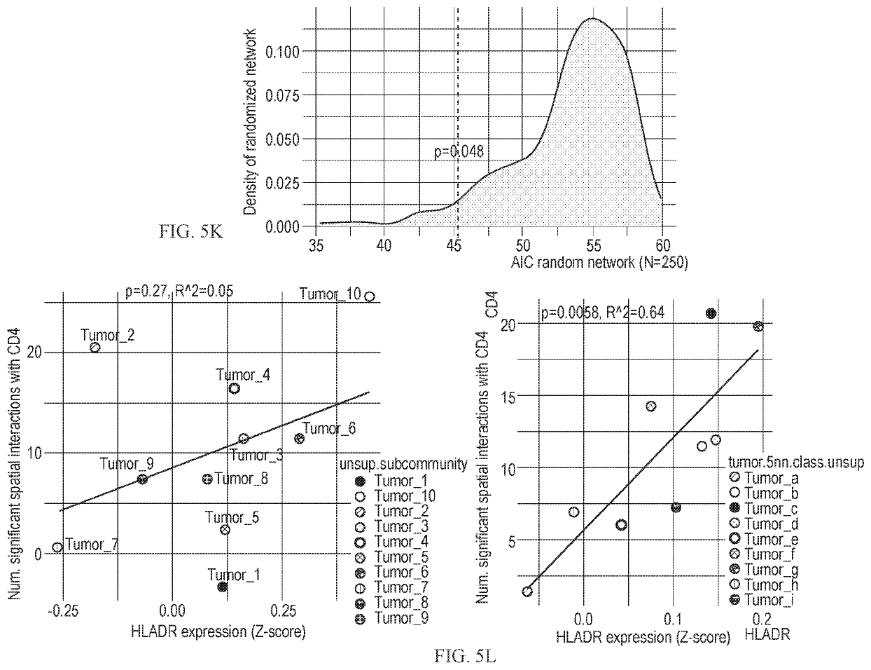

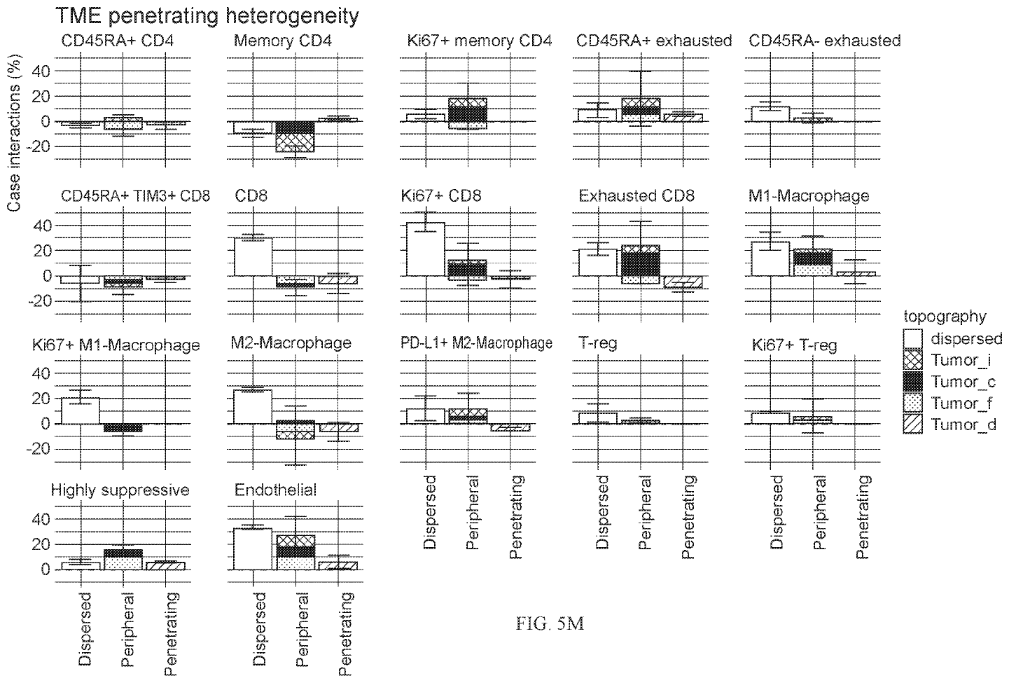

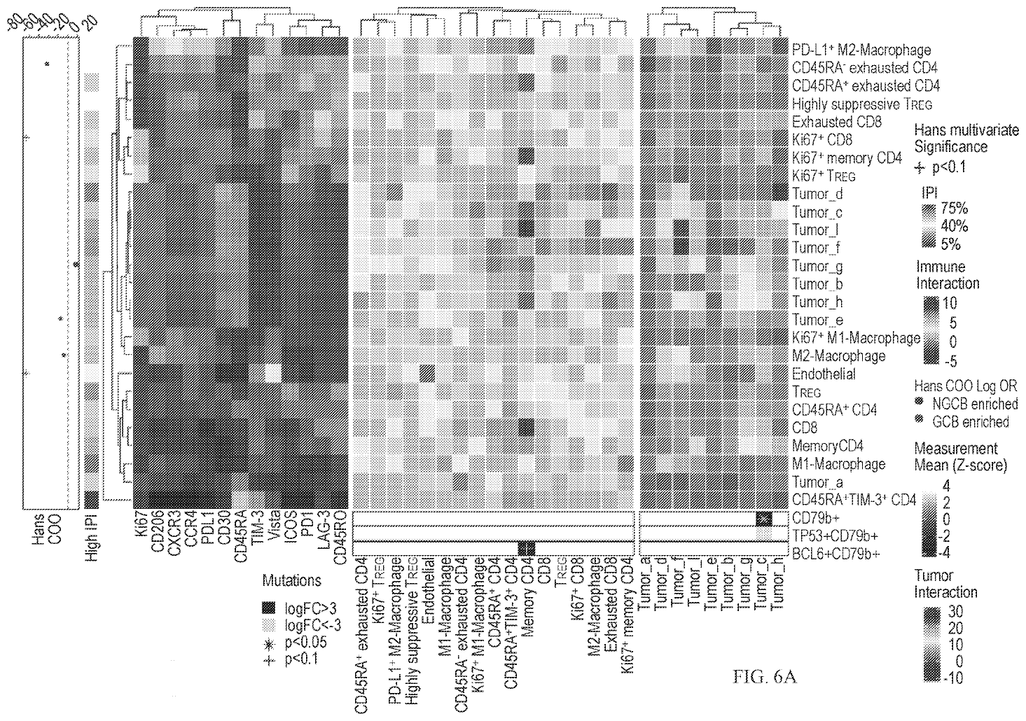

The histopathology of DLBCL is predominantly characterized by large sheets of tumor cells without a distinct structure, however closer inspection does reveal heterogeneity in the tumor cell and TME architecture. In order to characterize the single-cell topology we developed an algorithm for tumor cell-TME distance classification and tested it using synthetic images, and B cells in normal lymph nodes. The distance classification algorithm we developed classifies a selected population into sub-clusters based on distances to neighboring cell types (FIG. 5A). We constructed a demonstration set of four synthetic point patterns which simulated a lymph node (FIGS. 5G, 5H, 5I) and successfully classified a point pattern in terms of the nearest neighbor distance (NND) to the other synthetic types. Next, we classified B cells in a normal lymph node based on their distance relationship to other cells. The topology analysis identified B cell spatial clusters that corresponded to regions in the light and dark zone of germinal centers, the mantle zone of the follicle, and T cell rich paracortex regions (FIG. 5I).

Having validated the tumor-TME distance clustering approach on synthetic images and normal lymph node, we applied our distance classification algorithm to the tumor cells in DLBCL and identified nine spatial clusters, or tumor neighborhoods, based on tumor distance to the TME. The rarest topology was class “a”, or “tumor_a”, (0.93% prevalent), whereas tumor_f (21.1%), tumor_d (15.3%) and tumor_c (15.2%) were most common (FIG. 5D). The topology classes had significant heterogeneity across the immune proportions (p=0.01507; ANOVA) which is not unexpected since abundance of immune cells impacts their distance to tumor cells in a given tissue. From visual inspection it was clear that some tumor cells were dispersed among immune cells such tumor_b and tumor_e (FIG. 5D, inset 1). While other tumor cells formed tight layered clusters such as tumor_d, tumor_f, and tumor_i (FIG. 5D, inset 2).

Remembering that tumors are three-dimensional, we grouped the nine tumor spatial clusters into four groups inspired by the geologic structure of Earth: core, mantle, crust and dispersed. Tumor_d (average centroid distance to immune (dist)=47.9 μm) was defined as the tumor “core” because it was characterized by the furthest distance to the tumor-immune interface. Tumor_f (dist=30.9 μm) followed by tumor_i (dist=24.1 μm) were the next clusters in order and were defined as the tumor “mantle” because they were consistently adjacent to the core (FIG. 5D, inset 2). Tumor_c (dist=18.9 μm) and tumor_h (dist=18.9 μm) represented the boundary to the tumor-immune interface, or “crust” because it was adjacent to both the TME interface and mantle/core regions. Tumor_g (dist=13.8 μm), tumor_e (dist=13.7 μm) tumor_a (dist=13.2 μm), and tumor_b (dist=12.2 μm) had the shortest distances to the TME and formed unorganized tumor clusters (FIG. 5D, inset 1). Hence, we defined these scattered cells as “dispersed” regions of the tumor. Using this conceptual framework, we were able to spatially organize and interpret the tumor spatial clusters in terms of proximity to the TME.

The nine tumor spatial clusters were tested for association with COO using a multivariate logistic regression model. After adjusting for IPI, tumor_e (p=0.026), was significantly associated with non-GCB (FIGS. 5C, 5K). Tumor_e represented scattered tumor cells in regions rich with lymphocytes (FIG. 5D, inset 1). Tumor_c, found at the tumor immune interface was marginally associated (log-odds=0.46, p=0.08) with non-GCB (FIG. 5D, inset 2). These results are consistent with non-GCB tumors having increased interactions with immune cells.

To further explore if COO was associated with differences in tumor topology, we quantified the level of spatial clustering across each case, using the Clark-Evans aggregation index. The Clark-Evans aggregation index has been used in fluorescence microscopy studies and geographical research as a measure of spatial organization. Here, we used the standardized Clark-Evans index to compare the B cells in normal lymph node to the malignant B cells in GCB and non-GCB cases (FIG. 5E). The malignant B cells in GCB patients had similar spatial ordering to that of B cells in reactive lymph node (RLN) (Tukey adjusted p-value=0.47), whereas the malignant B cells in non-GCB had significantly different spatial organization (Tukey adjusted p-value=0.03). Comparing GCB to non-GCB, there was marginal difference in their spatial distribution (Tukey adjusted p-value=0.058). Further, as the spatial patterns of tumor cells become less clustered, the risk of dying decreased (Cox hazard estimate=−1.89, 95% CI: (−4.52, 0.75)). Increasing architectural disorganization is routinely recognized in carcinoma as a feature of tumor aggressiveness but is not routinely described in DLBCL histopathology. Our results quantify this feature and demonstrate that decreasing spatial regularity in DLBCL architecture correlates with both COO and survival.

We next explored whether different immune subsets had differential interactions with the tumor spatial clusters. To accomplish this, we determined for each of the major immune classes significant spatial attractions or repulsions, towards each tumor spatial cluster (FIG. 5F). We observed that CD4 T cells had significant associations within the tumor mantle/core compared to the crust/dispersed regions (OR=6.38, 95% CI: (2.91, 14.43), Fisher's exact p value=3.9e-06). This indicates that CD4 T cells were more likely to penetrate into tumor regions furthest from the TME interface. Whereas, the CD8 T cell had decreased association with mantle/core compared to the crust/dispersed (OR=0.16, 95% CI: (0.05, 0.41), Fisher's exact p value=5.6e-05), which indicates that CD8 are restricted to outer regions of the tumor cell-immune interface. Macrophages were able to partially penetrate into the mantle areas but were depleted from deeper core area (tumor_d) and had marginal decreased association at the tumor mantle/core compared to the crust/dispersed (OR=0.21, 95% CI: (8.5e-03, 1.1), Fisher's exact p value=0.12). Encouraged by these preliminary results, we next sought to comprehensively characterize the immune cells in each tumor zone.

### DLBCL Comprehensive Spatial Interactions Between TME and Tumor Topology, Functional State Profile, Mutations, and Patient Clinical Association.

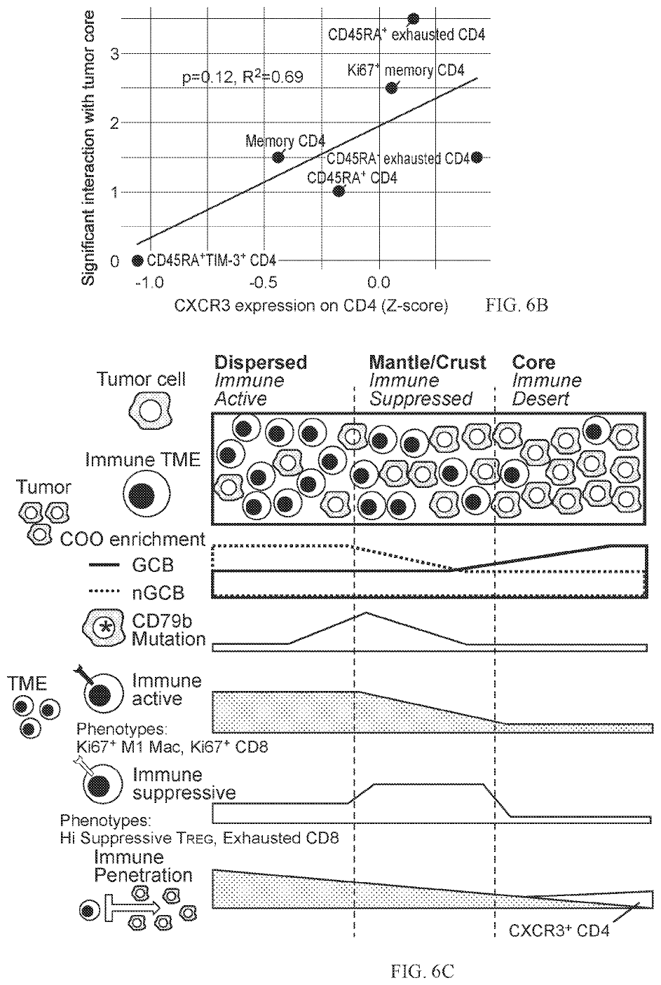

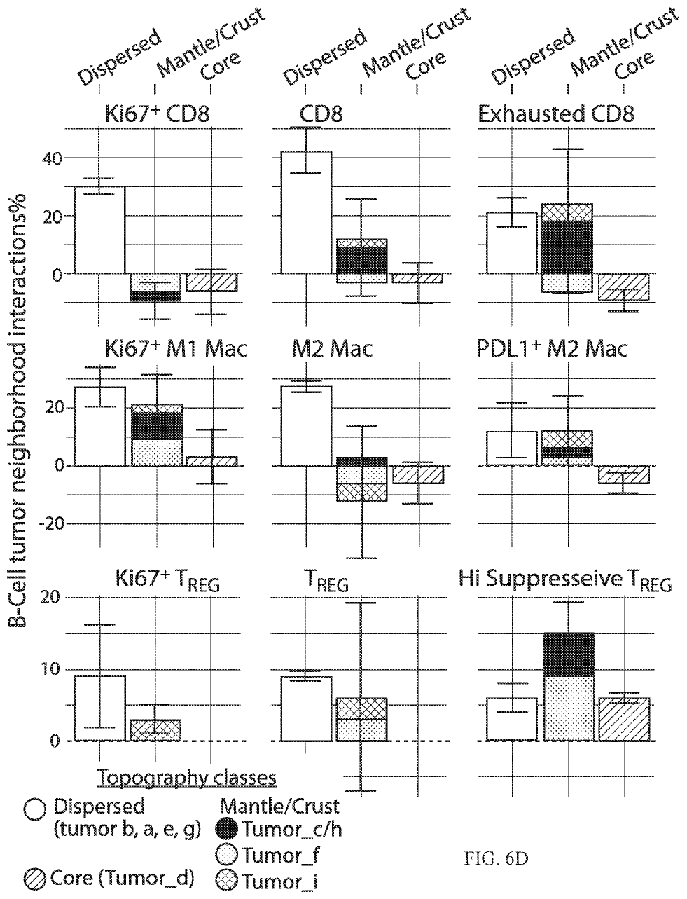

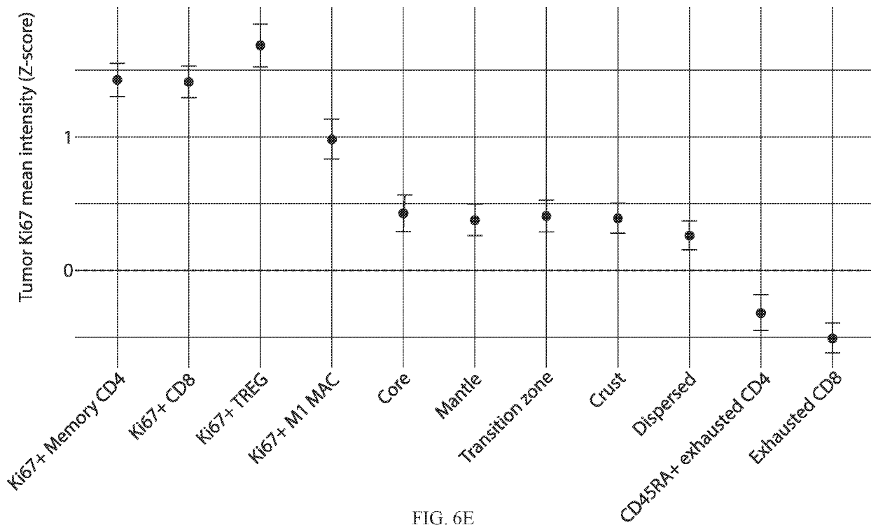

To comprehensively characterize the topology of tumor immune interaction, we adapted a previously described method for examining spatial interaction in IMC data to look for spatial interactions between tumor spatial clusters and immune phenotype sub-clusters within a 15 μm interaction zone. FIG. 6A summarizes the spatial interactions between immune phenotypes and tumor spatial clusters, the protein expressions on each cluster, as well as their multivariate association (IPI adjusted) with genetic mutations and Hans COO. For example, tumor_e, which was enriched in non-GCB subtype and was classified as a dispersed tumor type, had enriched interactions with activated and exhausted CD8 T cells, and exhausted CD4 T cells. While tumor_c, found at the tumor immune interface, showed significant association with CD79b mutation and TP53/CD79b co-mutation, and was enriched for interactions with highly suppressive T_{REG} and TIM-3+PD-1+LAG-3+ exhausted CD8 T cells.

Tumor_f, found in the inner mantle was enriched for endothelial cells, and depleted for CD8 subsets. This region was enriched for both highly suppressive T_{REG} as well as M1-macrophages and the heterogeneity may reflect the trafficking of immune cells from the peripheral blood. Tumor_d, which defined the core regions, contained a sparse TME neighborhood with depletion of most immune subsets with the exception of occasional CXCR3+CD45RA+CD4 and high suppressive T_{REG} cells. Tumor_d regions were not associated with refractoriness to chemotherapy however, the presence of these pockets of immune deserts may explain the lack of efficacy of checkpoint inhibitors in DLBCL and could have implications for cellular therapies such as CAR-T cells. While penetrating CD4 cells were overall rare, there was a moderate correspondence between CD4 and CXCR3 expression, and the total number of significant spatial interactions with the tumor core (p=0.12, R2=0.69, FIG. 6B). This indicates that CXCR3 plays a role in granting CD4 cells entry into otherwise restricted tumor sites and indicates a possible target for enhancing immune cell penetration into the tumor core regions.

To summarize our findings, we grouped the nine tumor spatial clusters into three zones: dispersed (tumor_b, tumor_a, tumor_e, and tumor_g), crust/mantle (tumor_c, tumor_h, tumor_f, and tumor_i) and core (tumor_d) and examined the immune cells surrounding tumor cells in each zone (FIG. 6D). The dispersed zone showed enrichment for proliferative subsets of CD8, macrophage and T_{REG}, surrounding tumor cells, which was consistent with high immune activity. The mantle/crust zone was most notable for enrichment of exhausted CD8 and highly suppressive T_{REG} around tumor cells indicating a zone of immune suppression. The core region was depleted for most phenotypes consistent with an immune desert. These three zones of immune activation, immune suppression and immune desert corresponded to the tumor spatial zones of dispersed, mantle/crust and core, respectively (FIG. 6C). On to this topology we overlaid COO and mutation status. Finally, by creating an ordered topology from high to low immune density we were able to identify CXCR3⁺CD4 cells as having the most tumor penetrating properties.

In this highly multiplexed single-cell study of the tumor immune architecture in DLBCL, we have quantified the intra-tumor heterogeneity of B cell lymphoma architecture and identified immune phenotypes that correlate with COO, tumor mutations, and response to therapy. This is the first such report in lymphoma where the high cellular density and the co-expression of markers on tumor and immune cells make this type of study particularly challenging.

Within DLBCL, a recent large-scale multiplexed study with a focus on the role of PD-L1 and PD-1 on CD20⁺ DLBCL cells was performed using immunofluorescence, but that study was limited to 13 markers. Xu-Monette et al. identified that PD-1⁺CD8 cells were associated with poorer overall survival. PD-1 expression on CD4 also showed adverse prognosis on univariate analysis at low cut offs, but a paradoxical better prognosis when a high level cutoff for PD-1 expression was used. These results indicate that there may be sub-populations within the PD-1⁺CD4 population that have differential effects on survival. Indeed, in our study we observed that CD4 and CD8 T cells with PD-1 expression could be further divided into activated Ki67⁺ T cells and terminally exhausted PD-1⁺ TIM-3⁺LAG-3⁺ triple positive T cells, highlighting the importance of highly multiplexed analysis. Furthermore, we demonstrated that subgroups enriched in refractory patients showed higher PD-L1 and PD-1 levels compared to CR, which is consistent with previous results.

Our multivariate analysis of tumor spatial clusters identified that exhausted CD8 T cells were excluded from tumor dense mantle and core regions while selected PD-1⁺CD4 T cells were better able to access the tumor core regions. This arrangement is somewhat reminiscent of the architecture of a germinal center with CD8 T cells relegated to the surrounding T cell zone and T follicular helper cells (PD-1⁺CD4) interacting with proliferating B cells in the center. In contrast to the normal GC, however, we also saw T_{REG} cells penetrating the tumor core regions which could provide an alternative explanation for the lack of infiltrating CD8 cells. Loss of MHC Class I expression has been previously reported in DLBCL (49%-61%) and could also contribute to a lack of CD8 T cell engagement with the tumor cells.

Our observation that tumor core areas, which exclude immune cells, are found in most DLBCL cases may have important implications for clinical failure of ICI therapies and resistance to cellular therapies such as chimeric antigen receptor (CAR) T cells. Among the CD4 cells, we observed that CXCR3-high subsets, albeit rare, had infiltrating potential in lymphoma. Importantly, Xu-Monette et al. reported that up-regulation of CXCL9, which is the ligand for CXCR3, was found in DLBCL with higher T cell infiltration, further supporting the role of CXCR3 in T cell homing to tumor core regions. We hypothesize that activating CXCR3 or enforcing expression of CXCR3 on CAR-T might improve immune cell penetration into lymphoma core regions. Applications of IMC analysis on treatment biopsies after CAR-T therapy could be particularly helpful to understanding treatment response and failure in this context.

Given the clinical failure of PD-1/PD-L1 inhibitors in DLBCL, identifying novel immuno-oncology targets for DLBCL remains important. DLBCL checkpoint therapy non-responsiveness can be further understood by investigating alternative check point molecules beyond PD-1/PD-L1 such as TIM-3, LAG3, and/or VISTA. Interestingly, DLBCL had higher levels of PD-1 on T cells compared to HL, an observation that is consistent with PD-1 being a poor biomarker of checkpoint response. Additionally, the comparison between DLBCL to HL identified TIM-3 as over-expressed primarily in CD4 and CD8 T cells which highlights it as a therapeutic target. Furthermore, PD-1 was enriched in T_{REG} in DLBCL, whereas TIM-3 on T_{REG} was not, which demonstrates the importance of identifying therapeutic targets that are differentially enriched on specific CD4/CD8 immune subsets. Given the high level of PD-1 on T_{REG} that we observed, anti-PD-1 antibodies may have led to increased activity of T_{REG} resulting in paradoxical suppression of immune response after check point therapy and clinical failure.

In order to perform our comparisons between DLBCL and Hodgkin lymphoma, we integrated IMC data from experiments performed using different antibody panels. The increased dynamic range of signal with IMC, compared to IHC, creates challenges with increased variability in signal intensity across experiments. Here, using approaches to normalize and standardize the data, we were able to demonstrate meaningful comparisons between different data sets.

In our study, within a relatively small part of the tumor area (approximately 1 mm²), we are able to discern a high degree of spatial and functional heterogeneity and describe a complex tissue architecture of immune response and suppression that corresponds to clinical feature. In our data, seven cases had at least one duplicate core on the TMA. Analysis by PCA indicated that most duplicates clustered together. This would indicate that small sample areas may be adequate to understand the complexity of tumor immune responses, at least in DLBCL. This is important because on-treatment and progression biopsies, which are critical to understand the kinetics of immune response and failure of immune-oncology agents, are often obtained as core needle biopsies, which are much smaller than diagnostic tissue samples.

In this study, diagnostic samples of patients prior to chemotherapy were analyzed, but not prior to treatment with immune-oncology agents. Since DLBCL responses to ICI are rare, we compared the TME in DLBCL to Hodgkin's lymphoma, where most patients achieve clinical responses.

Finally, the multiplex analysis provided by IMC could help guide the next generation of combination ICI therapies, including newer agents targeting CCR4, LAG-3, and TIM-3, or novel cellular therapies and bi-specific antibodies currently in development for lymphoma. Combining IMC with multi-omics profiling of pre-treatment and on-treatment biopsies will be powerful translational tools for understanding mechanisms of resistance and clinical failure for ICI and cellular therapies. In summary, IMC analysis of lymphoma reveals phenotypic and spatial structure in the TME that gives new insights to tumor immunology.

Tissue Microarray and Immunohistochemistry

Three of the six tissue microarray (TMA) blocks from the parent cohort with sufficient remaining tissues representative of viable tumor were obtained from the Pathology archive of Los Angeles County and USC Medical Centers. Immunohistochemistry (IHC) staining was performed on 4-μm tissue sections using DAKO ready-to-use antibodies with the EnVision FLEX and FLEX+ visualization systems (DAKO, Glostrup, Denmark) on an automated immunostainer (Autostainer Link 48, DAKO). Detailed IHC staining protocol and scoring methods have previously been published. TMA cores were 2 mm in diameter.

Imaging Mass Cytometry Staining

Three of the six TMAs with optimal quality of remaining tumor tissues from the larger cohort study were selected for this study. The TMAs contained 42 cores of FFPE DLBCL tissues from 33 patients and 2 cores from liver tissues. FFPE sections of 4-μm were baked at 60° C. for 90 minutes on a hot plate, de-waxed for 20 minutes in xylene and rehydrated in a graded series of alcohol (100%, 95%, 80% and 70%) for 5 minutes each. Heat-induced antigen retrieval was conducted on a hot plate at 95° C. in Tris-EDTA buffer at pH 9 for 30 minutes. After blocking with 3% BSA in PBS for 45 minutes, the sections were incubated overnight at 4° C. with a cocktail of 32 antibodies tagged with rare lanthanide isotopes obtained from Fluidigm (Table 2). Titration for PD-1 antibody was performed on tonsil tissue (follicular T helper cells in the germinal center). PD-L1 titration was done on a commercial slide containing formalin-fixed paraffin-embedded cell pallets of HDLM-2 (PD-L1+) and PC-3 (PD-L1−) cell lines from Cell Signaling Technology (Key resources table). Validation and titration of all other markers were done on control tonsil tissue. HLA-DR, pSTAT3, PD-L2 and Ephrin-B2 antibodies showed unspecific staining patterns in the control tonsil tissue and therefore were excluded from analyses.

Tissue Imaging and Ablation

All cores were evaluated by two pathologists to identify region of interest (ROI) on H&E. Slides were analyzed using the Fluidigm Hyperion Tissue Imager system that couples laser ablation with mass spectrometry. Laser beam of 1-μm² spot size was used to ablate tissue area of 1000-μm² per core at a frequency of 200 Hz. The metal isotopes were simultaneously measured and indexed against the location of each spot to generate intensities and digital spatial maps of the ablated tissues. Detailed descriptions of the ablation techniques have been previously described.

Image Analysis Pipeline

The ion counts for each metal-labeled antibody and slide location were compensated for the cross talk between channels then converted to OME-TIFF images. Images for each antibody were scaled using the 95 percentiles of the cumulative signal to remove hot spot pixels and normalized across acquisitions.

Image Segmentation

Channels representing distinct morphological features for cell nuclei (i.e. Ir193-DNA Intercalator, Histone H3, foxP3, Ki67) and membrane staining (i.e. CD8, CD68, CD45RA) were used for the Ilastik pixel classification training to predict nuclei, membrane/cytoplasm and background pixel class using cropped 2× scaled images. The probability maps were segmented using CellProfiler by subtracting the membrane probability map from the nuclei and then expanding the nuclei by 4 pixels.

Data Transformation and Normalization

The presented data used normalization such as the hyperbolic-arc-sine transformation, and Min/Max normalization at the 99^{th} percentile. The authors for Phenograph and t-SNE recommended using the 99^{th} percentile to clip the data to [0,1] scale and remove outliers, which we followed. For the cluster expression estimates, and spatial expression heterogeneity modeling, we used the mixed-effects linear model with the single-cell marker expression intensities scaled to a standard normal distribution across each ROI. Thus, for linear modeling we ensure that for all tissues, each marker expression was on the standardized Gaussian distribution. For Cox proportional hazards estimates, the relative proportions of each cluster were used as features, with survival times (N=30, events=7) in univariate models.

Analysis Workflow

The exploratory analysis used histoCAT, and downstream tSNE clustering using ‘Rtsne (v.0.15)’, ‘lme4 (v.1.1.21)’ R (v.3.6.3) packages for clustering and mixed-effects linear models.

Phenotypic Clustering

The images along with the masks were imported into histoCAT software for initial evaluation. Cell features were extracted and imported into R statistical software environment. We hierarchically performed meta-clustering to identify cell “phenotypes”. The first step under-clustered the data using lineage related markers (BCL2, BCL6, CD20, CD206, CD3, CD30, CD31, CD4, CD45RA/RO, CD68, CD8, EphrinB2, FOXP3 and HLADR) clustering each ROI (nearest neighbor, k₁=45) and then clustering on the centroids (nearest neighbor, k₂=15). The major cell classification per case identified 14 meta-clusters, and each ROI had on average 1,145.174 cells per meta-cluster. Quality control analysis performed Phenograph (k=50) on each major cell component separately to ensure that the major cell expression was homogeneous on the corresponding marker. Re-assignments of CD4 comprised of 1.54%, the initial tumor component reassigned 6.21% to CD8, CD4, MAC, and endothelial classes.

Following the re-assignments, we identified minor clusters for each major cell component by re-performing meta-clustering (k₁=45, k₂=15) on each TME component separately, with the inclusion of inducible state markers (cMYC, CCR4, CXCR3, Granzyme, ICOS, Ki67, LAG-3, PD-1, PD-L1, PD-L2, TBET, TIM-3, Vimentin, VISTA, and pSTAT3) and morphological features (Area, eccentricity, solidity, perimeter, percent touching, number of neighbors).

TMA and Replicate Divergence Analysis

The cohort comprised of 3 TMAs, and principal component analysis (PCA) was used to determine the presence of batch effects across TMAs. After annotating the 14 meta-clusters into major cell components, we performed PCA on the relative proportions per ROI and the PCA showed well-mixed visual representation of the variability of the data that did not have any TMA specific grouping. Case 17, 18, 21, 26, 27 and 31 had replicate ROIs taken and case 30 had triplicate ROIs. Using the R package ‘entropy (v.1.2.1)’, we computed the Kullback-Leibler (KL) divergence using a given replicate ROI relative frequencies per major cell component and compared a given replicate to the case relative average of the major components. The KL divergence scores per ROI were all below 0.10, however Case 26 had increased ROI heterogeneity (KL divergence of 0.26). The PCA analysis of the replicates showed minimal distance separations with the exception of Case 26. The replicates were mostly similar, with the exception of Case 26, and for the analysis of patient clinical variables we used the case averages across the ROIs.

Association Between Genetic Mutations, Cell of Origin and Proportions of Tumor-Immune Sub-Phenotypes

The cluster analysis followed the guidelines from the authors, which used the 99^{th} percentile normalization to remove outliers by scaling to relative 99^{th} percentile of each marker. The heatmap visualization standardized each ROI to a standard normal distribution. The heatmap depicts the mean normalized intensities, which ensures that the tissues were standardized. The clustering of all the phenotypes present used bootstrapping using ‘pvclust (v.2.2.0)’ and hierarchically clustered using sub-cluster means (Euclidean distance, Ward's method) dendsort (v.0.3.3)′ R package.

Clinical Association of Sub-Clusters to COO and Mutations

In order to test and measure the odds ratio between sub-clusters and HANS cell of origin classification of GCB and NGBC, we constructed a 2×2 contingency table at the patient/case level. The relative proportions of a given sub-cluster present/absent in each patient stratified on the HANS classification formed the 2×2 contingency table. The simple non-parametric odds ratio of association between the sub-clusters and HANS are depicted in FIG. 2A-2D and the p-values used the Pearson's Chi-square test of association. The HANS odds ratio depicted if the OR is greater than 1, or less than 0.90 with p<0.01. The odds ratio indicates the strength of association between a given cluster and GCB/NGBC classification.

The international prognostic index scores [0-5] were measured, and a median cutoff (>3) was used to identify patients with high IPI scores. For each sub-cluster the relative proportion of high IPI patients were computed providing a simple proportion of the relative proportion of high IPI subjects present within a sub-cluster.

BCL2 and MYC protein were measured by immune-histochemistry. BCL2 overexpression was measured using a 40% cutoff judged by IHC, and MYC over expression was determined as 70% threshold. Patients classified as double expressors were identified as BCL2 above the 40% and MYC above the 70% threshold. A logistic regression was used to regress the case relative frequency of each sub-cluster onto the patient clinical indicator variable for double expressor. Tumor_6 (OR=2.061), Tumor_4 (OR=3.01) and CD8_4 (OR=2.54) had odds ratio of association between the sub-cluster and double expressor greater than 2, with p<0.01.

We obtained the molecular variant/mutations of a limited gene panel (Cancer genetics Inc.) in 22 subjects in this cohort and focused the mutation model on influential genes previously reported in lymphoma. Subjects with a variant on a specific gene such as BCL2, BCL6, CD79b, TP53, SGK1 EZH2, NOTCH1/2, and MYD88 amino acid L265P were identified. Co-occurrence between mutations were studied using a Boolean set formation separating mutations that co-occurred or occurred independently. TP53 (n=8) occurred independently in 3 subjects, and co-occurred with CD79b in 4 separate subjects, and once with SGK1. Of the 4 TP53+CD79b+, one subject had an additional variant in MYD88^{L265P}. BCL2 (n=3) co-occurred once as an independent mutation, once with BCL6 and EZH2, and once with only EZH2.

CD79b variant (n=6) occurred once with BCL6, 3 co-occurred with TP53, 1 with TP53 and MYD88^{L265P} and 1 as an independent mutation. CD79b did not co-occur with EZH2.

To compute the log-odds (FIG. 2B), the estimates were derived using epitools' R package, to create a 2×2 contingency table of with the rows corresponding to a specific mutation present (+) or not present (−), and the columns corresponding to the counts of cells corresponding to NGBC or GCB. from only the 22 subjects that had mutation screening. For FIG. 2C) we fit an empirical Bayes linear model (eBayes) from limma (v3.40.2_′ R package using patient mutational features in a design matrix, and case relative proportions (%) for each sub-cluster from FIG. 2A. We then used Benjamini-Hochberg multiple test corrections, to identify clusters that were differential for the given mutation.

Estimation of Driver Proteins on Sub-Cluster

We avoided calling a sub-cluster immune subset “positive” or “negative” by arbitrary thresholding but used a linear mixed-effects model to derive effect estimates of markers across all sub-clusters. We constructed the linear model using the ROI standardized single-cell expression by mapping all cells per ROI to a standard normal distribution. Hence all ROIs had standardized marker intensity, and for generalizability all estimates excluded non-treated subjects or subjects lost-to-follow-up. We provided point estimates using complete data only. The point estimates and 95% confidence intervals on key markers were derived using (‘lme4 (v.1.1.21)’, and ‘multcomp (v.1.4.12)’ R packages). The point estimates for “driver”, or protein enrichments, on sub-clusters were performed using mixed-effects linear model which treated the case as a random effect, and the sub-cluster mean expression per ROI as the features.

We created custom contrasts for all sub-cluster average expression and tested for enrichment by comparing to the global mean for that protein in a single model. The significance of a particular sub-cluster is interpreted as significantly higher/lower to the grand average of that protein (p<0.05), which determined “+/−” for that cluster. The confidence intervals were generated using general linear hypothesis test (‘glht’) function from the R package multcomp (v.1.4.12)′ and derived from ROI standardized values which we hope increases reproducibility in this field.

Sub-Cluster Association with Chemotherapy Response and Refractoriness.

The Barnes-Hut t-stochastic neighborhood embedding (tSNE) was constructed using (‘Rtsne (v.0.15)’ Package), and UMAP (umap v0.2.2.1) were used. After performing the UMAP using all single-cells, manual annotations of the sub-clusters were performed by visual inspection. The tSNE parameters were initial dimensions: 50, perplexity: 30, theta: 15. In order to compare enriched/depleted immune subsets, the case proportions for each sub-cluster were derived (%), as well as the standard error of the mean 95% confidence interval (N=33) by averaging ROIs at the case level. The p-values for proportion enrichment were computed using simple linear regression (FIG. 3E, 3F). In order to test enrichment of LAG-3 comparing REF to CR, we fit a One-Way-ANOVA using a mixed effects model and tested pairwise contrasts for each immune subset.

A univariate Cox proportional hazards model computed the Cox hazard estimates and their 95% confidence interval (FIG. 4A, and related to FIGS. 5A-5F). The response included the survival time-to-event (events=7), and the features were the case level proportions of all sub-clusters, and the additional feature “Tumor Spatial Regularity” corresponding to the Clark-Evans aggregation index discussed in FIGS. 5A-5F.

For the neighborhood enrichment comparing refractory to response (FIGS. 3D and 6A), we performed neighborhood analysis on the full cohort and identified significant interactions using 1,000 permutations which generated the null distribution and a significance threshold of 0.025. For FIG. 3D, from the reference of tumor we computed the total significant interactions of all immune phenotypes as a total proportion. The response status relative proportion of significant interactions were depicted along with the 95% confidence intervals. The similar tumor neighborhood summary case proportions were computed similarly for FIG. 6B.

Cross-Cohort Analysis of DLBCL and Hodgkin Lymphoma (HL)

To compare the TME in both diseases, we subset both experiments to include only CD4, CD8, Macrophages, and TREGs, and omitted all other phenotypes present in either experiment. The TME expression of integrated data including both DLBCL and HL identified primary phenotypes in the TME in both diseases. The HL data was obtained from our recent publication in Hodgkin's lymphoma that included 5 lymph nodes, 22 cases of HL (1 lymphocyte rich, 9 mixed cellularity, and 12 nodular sclerosis subtypes). This experiment was performed with a 36 panel list, of which 21 overlapped with the DLBCL experiment (CCR4, CD206, CD20, CD30, CD3, CD45RA, CD45RO, CD4, CD68, CD8, CXCR3, EphrinB2, FOXP3, HLADR, ICOS, Ki67, LAG3, PD-1, PD-L1, TIM-3, and TBET). The meta-clustering (k₁=45, k₂=15) process identified 17 HL meta-clusters using Min/Max normalization. To compare the TME expression in the 75 ROIs from both diseases, we subset both experiments to include only CD4, CD8, macrophages, and T_{REG}s. Prior data integration, we first selected markers that had minimal differences per ROI across by visual inspection of the ROI interquartile ranges across both diseases. Markers such as CD4, CD8, CD68, FOXP3, CD206, DNA, PD-L1, PD-1, TIM-3, CCR4, CXCR3, ICOS, Ki67, and cell area were selected because they indicated similar inter-quartile ranges and most constant means across experiments. To integrate the single-cell data, we first standardized the expression to-standard normal, and then used ‘limma (v.3.40.2)’ to fit an empirical Bayes linear model and regress out the batch effects related to the 2 experiments at the single-cell level (‘sva’ (v.3.32.1) package). To evaluate the batch effects, we used DNA, and the area of the cell objects as a positive control because we should expect that DNA content, and immune cell areas should be very similar across experiments. After data integration, we observed consistent area sizes across experiments and DNA expression. In contrast, LAG-3 failed to integrate and had dissimilar dynamic range. and was excluded. PCA, at both the case level and single cell level, along with uniform manifold mapping (uMAP) at the single cell level were performed to visually inspect the presence of batch across diseases.

After selecting markers and performing integration, we used the k-nearest neighbor batch-effect correction test (‘kBET’ R package (v.0.99.6)) to quantitatively measure the batch effect. This test is a metric to measure the quality of the batch correction and performs a Pearson's χ² test comparing a randomly sampled subset to measure the association to the levels of batch covariates. The Pearson's χ² assumes that the data are inter-mixed, and the kBET returns a metric of the average rejection rate of the null hypothesis across the iterations (number of iterations=100) of sampling. Hence a low kBET score, corresponds with a low rejection of the null hypothesis in the χ² test which assumes that the data are well inter-mixed. Therefore low kBET scores corresponds with low batch effects. The kBET scores across all phenotypes yielded 0.2422 score, and CD4 had the lowest kBET score (kBET=0.1484, p-value=0.31), whereas TREGs had the highest batch effect score (kBET=0.37, p=0.08). Note that we tested for batch on each TME component separately and did not adjust the p-values for multiple test corrections. The input for the kBET metric where the 75 unique ROIs across both experiments (rows) and the columns were the Z-transformed selected protein measures previously described.

We examined the explained variance of the selected integrated expression markers by the phenotype levels (CD4, CD8, MAC, TREG), or the disease type (HL or DLBCL). For a well-integrated experiment, the integrated expression should have a low explained variance associated with disease type. We performed a Two-Way ANOVA regressing the protein expression onto categorical features such as phenotypes (4 levels) or disease type (2 levels).

For PD-1, we observed that the phenotype categorical variable was a significant explanatory variable (F-statistic=15.38, p-value=0.025), whereas the experimental factor was not (F-statistic=1.32, p-value=0.33). Similarly, PD-L1 the phenotype categorical variable was a marginal explanatory variable (F-statistic=4.56, p-value=0.12), whereas the experiment feature was not (F-statistic=0.02, p-value=0.88). For TIM-3, the phenotype feature explained more of the variability of TIM-3 (F-statistic=2.73, p-value=0.22) compared to the experimental factor (F-statistic=0.06, p-value=0.82). The ANOVA indicated that the average expression had more association with the variability across phenotypes levels as opposed to experimental/disease type.

In order to compare proportions of the TME across disease types, the case relative proportions (%) were tested using simple linear regression, and the p-values were multiplied by 4 using Bonferroni multiple test corrections, using significance threshold of 0.05 as the type-1 error rate. We compared the integrated expression of 3 (TIM-3, PD-1, PD-L1) proteins at the patient level average and used a Student's t-test to compare case average mean differences.

The joint TME unsupervised clusters were annotated (FIG. 4G) using+ threshold calls of 0.49-0.50 for calling positivity, and cross-checking using the ANOVA model. The annotated heatmap of the labels are depicted in FIG. 4A. The cluster proportion enrichment (DLBCL/HL) were measured using logistic regression of the disease type regressed onto the cluster proportions using univariate model, and p<0.01 alpha level threshold.

Spatial Classification and Topology of DLBCL

Infiltrating lymphocytes could be defined by using the centroid (the average of 5 nearest neighbors (NN) Euclidean distance to cancer cell) which identified a lymphocyte that resided inside a convex hull (domain) of 5 neighboring tumor cells. The 5-NN centroid provided additional information compared to the first nearest neighbor because if a T-cell had shorter distance to the centroid, compared to the 1-NN, this implied infiltration because the lymphocyte was in closer inside the tumor domain. We sought to develop an algorithm which would classify the tumors by their proximity (5-NN centroids) to nearest immune cells and create a linear ordering by distance. Importantly, the tumor ordering in the context to TME proximity represented a contour immunographic map, where furthest distance to the nearest immune cell was analogous to low immune infiltration potential (“steep valley”), whereas tumors that were immediately co-localized to immune cells would have increased immune potential (“top-of-hill”).

In order to develop the algorithm, we generated 4 synthetic point patterns using spatstat (v.1.59.0)′ R package (FIGS. 5G, 5H). The synthetic image 1 and 2, had three pattern types, which simulated a germinal center (pattern 1), and the T-cell zone (pattern 2), we included a ‘null’ type (pattern 0) which was included in the distance algorithm, but a shape was left unassigned. The synthetic image 3 had four pattern types. Whereas synthetic image 4 had only two pattern types which demonstrated how the algorithm would order a pattern in terms of distance to the interface (pattern B). We further tested the distance classification algorithm on 6 reactive lymph nodes (FIGS. 5I, 5J). From the raw ablation images (FIG. 5I) we observed that the CD20 follicle, which captured light/dark zones, was subset (FIG. 5I) into sub-types corresponding with nearest distance to PD-1+TFHs and other T-cells in the paracortex region.

The algorithm simply computed the average distance to the 5-NN (centroids) from each B-cell toward the other immune phenotype, and then used Phenograph to meta-cluster (k₁=45, k₂=15), the distances into classes which were then ordered. The distance centroids for each tumor cell were used with Phenograph algorithm to classify the tumor cells into clusters dependent on their centroid distance to the immune cells, which provided an immune contour. We observed that the centroid distances were linear to 1-NN neighbors.

Multivariate Logistic Regression of Topology Class Abundances in DLBCL

The multivariate logistic regression was performed on the topological relative case tumor proportions (%) that included the IPI scores (FIGS. 5C, 5K). We compared the Akaike Information Criterion (AIC) from the multivariate regression model to the AIC from the randomized model which permuted the topology labels 250 times to generate a null distribution for the AIC for comparison. Note for the tumor topological multivariate regression tested the multivariate model of all the topology case proportions after adjusting for IPI. We reported the log-odds of the coefficient estimates along with the coefficient p-values.

Spatial Organization of Tumor Topology in DLBCL Comparing COO and Reactive Lymph Node

The Clark-Evans aggregation index is used to measure spatial organization of a point pattern and was performed using the spatstat (v.1.59.0)′ R package. We compared the Clark-Evans standardized indices at the ROI level in DLBCL and 6 lymph nodes and compared them using a Student's t-test (Tukey test for multiple comparisons). We did not include tumor_h nor tumor_e into the spatial organization model, because their abundances were highly associated with GCB (p=0.057) and NGBC (p=0.026). Tumor classes that were 10% prevalent were selected, and rare topography classes (tumor_a (0.93%), tumor_b (6.9%)) were excluded. By visual inspection, classes: “c” (15.2%), “d” (15.4%), “f” (21.1%), “g” (11%), and “i” (14.9%) were co-localized, however statistical significance was achieved after dropping tumor_d from the spatial organization model, however the trends were still observed.

Neighborhood Analysis

The neighborhood analysis depicted in FIG. 6A used the algorithm deposited by Bodenmiller group, and significance was determined using 1,000 permutations and interaction distance of 15 μm. Significant interactions used p<0.025 to determine a significant signed interaction. The neighborhood pairwise interactions were performed for all pairs, and the significant signed interactions were depicted as a heatmap along with the corresponding mean normalized intensity, association with clinical features, and mutations.

The associations to GCB/NGBC were measured using one multivariate logistic model which used the NGCB/GCB (I/O) as the response and the case level abundances of immune subsets and tumor topological class as the features and included IPI adjusting for IPI. The immune subsets were selected using LASSO regression (using lambda 1s^{t} standard error penalty) (FIG. 6A left dot plot) and the log-odds of the multivariate model using only the selected features are depicted.

Multivariate Clinical Associations Between Sub-Clusters, Topological Clusters Using Multivariate Logistic Regression

The first figure (FIG. 2A) applied a simplistic non-parametric test of association as a univariate measure of strength of association between sub-cluster proportions, GCB/NGBC and double-expressors. The FIG. 6A, however included a multivariate logistic regression for each TME component (CD4, CD8, MAC, T_{REG}, tumor topology).

The FIG. 6A depicts significant clinical associations identified from the multivariate model of all selected immune subsets from CD4, CD8, macrophages, and TREGs, with the inclusion of IPI and tumor topology classes selected from the package glmnet (v2.0.18). For mutational association, we fit the tumor topology case proportions to an empirical Bayesian linear model using patient mutational features in the design matrix. Statistically significance was determined after using Benjamini-Hochberg multiple test corrections (q<0.05). We also fit a global mixed-effects linear model to identify if any tumor topologies significantly co-expressed cMYC/BCL2/BCL6 but did not identify cMYC as significantly expressed.

Spatial Communities and TME Quantification of Penetration

The neighborhood analysis identified all pairwise interactions (p<0.025) and note that not all spatial interactions are symmetric. In order to describe the tumor topology neighborhoods, we summarized the TMEs from the topology classes, and toward the immune phenotypes. The total sum of the signed interactions in the outer/dispersed (topologies: b, e, g, and h), the tumor periphery (topologies: c, I, f) and tumor core (topology: d) were computed. We defined semi-penetrating as TMEs significantly attracted to the periphery zones and penetrating as TMEs significantly attracted to the tumor core. The 95% confidence intervals were computed using the signed interactions, and the Student t-distribution (d.f.=7).

### Example 2

Provisional patent application No. 62/905,980 is hereby incorporated by reference. Detailed examples are shown below, including figures from FIG. 7A to FIG. 14G.

We ablated 41 tumor cores from 33 DLBCL cases, which included 33 primary cores. In order to standardize the core selection, only the first cores from the duplicates were included in the main analyses. Single-cell segmentation of IMC data yielded an average of 16,542 cells per ablated core, which was sufficient for downstream analyses and comparable to that reported in other mass cytometry studies. Markers that define cell types or lineages were classified as “phenotypic” markers, whereas those that define cell states or are inducible were classified as “inducible” markers (Table 2). Phenograph was used to cluster cells based on similarities of their phenotypic marker expression as described by Levine et al. The unsupervised clustering algorithm identified 14 clusters which were categorized as tumor (6 clusters), CD4 (3 clusters), CD8 (2 clusters), T_{REG} (1 cluster), macrophage (1 cluster), and endothelial cells (1 cluster) based on phenotypic marker expression. Then meta-clusters were generated by re-phenographing the centroids of each subpopulation (FIG. 1A). The TME across the whole cohort was composed predominantly of CD4 (36%), CD8 (30%), and macrophages (26%). T_{REG} (8%) were generally rare (FIG. 1B). Immune cell infiltration in individual tumor samples ranged from 7% to 75% (FIG. 1D, and analysis of the TME composition revealed marked heterogeneity in the distribution of immune subsets across cases (FIG. 1E). Interestingly, we observed a significant positive correlation between the proportion of immune cells and CD4-to-immune ratio (R=0.53, p=0.001, FIG. 1F), whereas the ratio of MAC-to-immune decreased with increasing proportion of immune cells (R=−0.38, p=0.03, FIG. 1G).

TABLE 2
Antibody panel. Related to STAR Methods.

| | | | Target/ | |
|---|---|---|---|---|
| N | Isotopes | Categories | Antibody target | Clones |
| 1 | 161Gd | B cells | *CD20 | H1 |
| 2 | 170Er | Pan T cells | *CD3 | poly-C-term |
| 3 | 156Gd | T Helper | *CD4 | EPR6855 |
| 4 | 145Nd | Th1 | *T-bet | D6N8B |
| 5 | 162Dy | Cytotoxic T cells | *CD8a | C8/144B |
| 6 | 163Dy | Regulatory T cells | *Foxp3 | 236A/E7 |
| 7 | 155Gd | Naïve T cells | *CD45RA | HI100 |
| 8 | 173Yb | Memory T cells | *CD45RO | UCHL1 |
| 9 | 167Er | Cytotoxic granule | ⁺Granzyme B | EPR20129-217 |
| 10 | 169Tm | M2 Macrophage/APC | ⁺CD206 | 5C11 |
| 11 | 159Tb | Macrophage/APC | *CD68 | KP1 |
| 12 | 174Yb | MHC-II, Dendritic cells | *HLA-DR | YE2/36 HLK |
| 13 | 148Nd | Immunoregulatory | ⁺ICOS | D1K2T |
| 14 | 150Nd | Immunoregulatory | ⁺CD274 (PD-L1) | 28-8 |
| 15 | 151Eu | Immunoregulatory | ⁺CD134/OX40 | Poly |
| 16 | 153Eu | Immunoregulatory | ⁺LAG-3 | D2G40 |
| 17 | 154Sm | Immunoregulatory | ⁺TIM-3 | D5D5R |
| 18 | 160Gd | Immunoregulatory | ⁺Vista | D1L2G |
| 19 | 172Yb | Immunoregulatory | ⁺PD-L2 | 176611 |
| 20 | 175Lu | Immunoregulatory | ⁺CD279 (PD-1) | NAT 105 |
| 21 | 142Nd | Immunoregulatory, Th1 | ⁺CD183 (CXCR3) | G025H7 |
| 22 | 149Sm | Immunoregulatory, Th2 | ⁺CD194 (CCR4) | 205410 |
| 23 | 146ND | Oncoprotein | ⁺BCL2 | EPR17509 |
| 24 | 147Sm | Oncoprotein | ⁺BCL6 | K112-91 |
| 25 | 164Dy | Oncoprotein | ⁺C-MYC-P67 | 9E10 |
| 26 | 158Gd | Transcription factor | ⁺pStat3 [Y705] | 4/P-Stat3 |
| 27 | 168Er | Proliferation | ⁺Ki-67 | B56 |
| 28 | 143Nd | Stroma | *Vimentin | RV202 |
| 29 | 144Nd | Vascular | *CD31 | C31.3 |
| 30 | 152Sm | Vascular | *CD34 | QBEnd/10 |
| 31 | 166Er | Vascular, immunoregulatory | ⁺EphrinB2 | EPR10072 (B) |
| 32 | 176Yb | Nuclei | Histone 3 | D1H2 |

*Phenotypic markers
⁺Inducible markers

CD4 and CD8 T cell densities have been shown to be associated with favorable outcomes in DLBCL. We calculated CD4, CD8, T_{REG}, and macrophage abundance relative to total number of immune cells and studied the prognostic impact of these immune subpopulations on OS using Cox proportional hazards model and Harrell's Concordance statistic or C-index, which measures the general predictive power of the regression model. A model with C-index of ≥0.7 is generally considered to have good predictive power. Survival analyses showed no correlation between OS and the abundance of CD4 (p=0.52, C=0.34), CD8 (p=0.59, C=0.45), T_{REG} (p=0.82, C=0.32) and macrophages (p=0.88, C=0.33). However, we hypothesized that sub-groups of immune cells within each major compartment might have prognostic impact.

### Survival Analysis Using M-Score Shows Complex Tumor Phenotype Co-Expressing PD-L1/TIM-3/CCR4 to be an Independent Predictor of Poor OS in DLBCL.

To study the prognostic impact of complex tumor and immune phenotypes in DLBCL, we examined the expression of all inducible immuno-regulatory proteins on the previously identified phenotypes (FIG. 1A) and correlated them with OS. A common approach to studying the significance of biomarker expression is to define positive and negative thresholds. Carey et al used pathologist-defined subjective threshold for calling a marker positive. Keren and colleagues arcsine transformed intensity values to define positive and negative cells. Using a similar procedure and cut off of 0.5, we examined the expression distribution of all markers and found that normalized intensity for phenotypic markers followed bimodal distribution allowing separation between negative and positive calls at the chosen cut point (FIG. 7A and FIG. 13A. Moreover, using this cut point, we showed reasonable distribution of phenotypic markers' positivity on the tumor and immune populations previously identified by meta-clustering (FIG. 7B and FIG. 13B. However, for certain inducible markers such as PD-1 and CXCR3, it was more challenging to determine the optimal cut point because their expression was distributed either below or above 0.5 (FIG. 7A and FIG. 13A).

To tune the PD-1 cut-point, we assumed the abundance of PD-1+ T cells on the exhausted T cell phenotypes (TIM-3+/LAG-3+CD4, CD8 and T_{REG}) should be higher than on non-T cells such as macrophages and tumor cells. The optimal cut point of 0.35 gave the highest PD-1 abundance ratio on exhausted T cells compared to macrophages and tumor cells.

Most previous studies on PD-L1 in DLBCL defined PD-L1 positivity threshold using immunohistochemistry (IHC) and reported discordant results both in the proportion of positive cases and the prognostic impact of PD-L1 positivity. Depending on positivity threshold, reported PD-L1 positivity on tumor cells ranges from 5% to 54% of the cases. Some investigators found PD-L1+ tumors to correlate with poor clinical outcomes, while others reported no association between tumor PD-L1 positivity and survival.

Since there is a great deal of discrepancy in the literature about the levels of PD-L1 expression found in DLBCL and its prognostic impact, we paid particular attention to establishing the optimal thresholds for PD-L1. The cases in this cohort had been scored by expert pathologists for PD-L1 expression using 30% positivity threshold as previously described. Based on scoring results from that work, 24% of the tumors in the sub cohort were PD-L1 positive by IHC. Using 0.5 cut point for PD-L1 intensity and 30% as threshold for case-level positivity, however, only 3% of the tumors were positive for PD-L1. We, therefore, used the PD-L1 IHC and RNAscope results to guide PD-L1 tuning of the PD-L1 threshold. At 0.34, the linear model which regressed the PD-L1+ relative abundance onto IHC (F=25.63, adjusted R²=0.12, p<0.0001), and RNAscope scores (F=8.02, adjusted R²=0.04, p=0.006) had the highest average F-values (FIG. 7C). Using the optimal 0.34 cut point for PD-L1 intensity and 30% threshold for classifying positive cases, we showed PD-L1+ tumor in 82% of our DLBCL cohort, which is higher than PD-L1 positivity previously reported.

To identify relevant immuno-regulatory markers for the assessment of prognostic impact of complex tumor and immune phenotypes, we performed exploratory analyses looking at the association between OS and the abundance of each inducible marker. Markers were considered having significant influence on OS if likelihood ratio test (LRT) p-value is <0.05 or Harrell's C-index is ≥0.7. The analyses identified PD-L1 (p=0.3, C=0.74), TIM-3 (p=0.06, C=0.76) and CCR4 (0.007, C=0.66) as predictors of OS in univariate model. We then looked at the distribution of complex immune and tumor phenotypes, which co-expressed at least 2 of these markers, in our cohort. PD-L1/TIM-3/CCR4 triple positive CD8 and CD4 represented the two most dominant T cell subsets in the TME. They accounted for 21.33% and 16.57% of all CD8 and CD4 T cells, respectively (FIG. 7D). Survival analyses showed a trend for association between high abundance of PD-L1/TIM-3/CCR4 triple positive CD8 T cells and poor OS (p=0.08, C=0.77). Complex phenotypic analysis also showed that 76.06% of PD-L1/TIM-3/CCR4 triple positive tumor cells expressed BCL2 (FIG. 7E). The BCL2+PD-L-1/TIM-3/CCR4 triple positive tumor cells made up 10.02% of all tumor cells and were significantly associated with refractory disease compared to the BCL2+PD-L1/TIM-3/CCR4 triple negative tumor cells (χ²=5.62, df=1, p=0.01). More importantly, survival analysis revealed higher abundance of PD-L1/TIM-3/CCR4 triple positive tumor cells to predict inferior OS in DLBCL (p=0.003, C=0.80, FIG. 7F).

To evaluate how the choice of cut point affected our ability to predict clinical outcome in DLBCL, we performed survival analyses on tumor and TME cell phenotypes expressing PD-L1, TIM-3 and/or CCR4 uniformly at 0.3, 0.4, 0.5 and 0.6 cut points. Except for the lowest cut point of 0.3, the predictive power of various CD4 and CD8 phenotypes was largely preserved across a range of cut-points demonstrating that these finding are robust and affected by the choice of cut-point (FIG. 14A-FIG. 14F).

A challenge in studying biomarker expression in the TME using IHC is that the technique is only semi-quantitative, while IMC data are highly quantitative with dynamic range of over 4 logs of expression Inspired by H-score, which considers both the staining intensity and the proportion of stained cells, we calculated an intensity-weighted abundance of each inducible marker, denoted as “M-score.” We hypothesized that the M-score would allow us to make better use of the quantitative nature of IMC data, while eliminating the use of arbitrary thresholds of marker positivity. Survival analyses using M-scores showed similar association trends for all phenotypes as in cut-point-based analyses (FIG. 14A-FIG. 14G). Re-assessment of the prognostic impact of complex tumor phenotype using M-score showed negative association between OS and M-score for tumor cells co-expressing PD-L1/TIM-3/CCR4 (p=0.003), the same trend as observed in the analysis using the optimal 0.5 cut point (0.34 cut point for PD-L1). The C-index improved from 0.80 in cut-point-based model to 0.85 in the model using M-score in univariate model (FIG. 7F and FIG. 7G). These results indicated that M-score performed at least as well as the optimal cut points and could serve as an independent non-parametric alternative for studying the prognostic impact of both complex and non-complex phenotypes in DLBCL and remained highly significant after adjusting for International Prognostic Index (IPI) scores (p=0.003, C=0.83, FIG. 7H).

Incorporation of CD8 Spatial Neighborhood Feature with Phenotypic Profiling Improves Survival Prediction in DLBCL.

A key advantage of IMC over other multiplex methods such as suspension CyTOF or scRNAseq is the preservation of information about the tissue architecture within the tumor microenvironment. We hypothesized that studying immune cells within their local neighborhoods could give us insight into the complexity of immune and tumor cell interaction. Furthermore, we hypothesized that spatial “filtering” would allow us to focus on immune/tumor interactions that were more likely to be biologically relevant.

Previous studies, including one on the larger parent cohort of this study, have shown high PD-L1 expression on malignant B cells to be associated with poor survival in DLBCL. Univariate survival analysis using M-score showed similar association trend though the significance level was not reached (p=0.27, C=0.73, FIG. 8A), likely due to lost statistical power in our sub-cohort. Since PD-L1 on tumor cells plays an important role in preventing cell-mediated killing, we revisited the prognostic impact of tumor's PD-L1 expression in the context of their proximity to CD8 T cells. We applied a “spatial-filter” by limiting analysis to the five nearest (5NN) tumor cells for each given CD8 T cells. When considering only tumor cells that neighbored CD8, a stronger association was observed between higher M-score of tumor cells expressing PD-L1 and worse OS (p=0.06, C=0.79, FIG. 8B).

Rodig and colleagues showed that endothelial cell expression of PD ligands could inhibit CD8− mediated anti-tumor immune response. Therefore, we studied the expression of PD-L1 on endothelial cells and assessed their impact on clinical outcome in DLBCL. We found an association between higher M-score for endothelial cells expressing PD-L1 and worse OS (p=0.06, C=0.76, FIG. 8C). When proximity to CD8 was accounted for, however, the strength of this correlation between OS and PD-L1-expressing endothelium increased significantly (p=0.001, C=0.80, FIG. 8D).

CD8 Spatial Analysis Yields Insights into the Functional Statuses of Immune Cells in the TME and Reveals Distinct Types of CD8 Neighborhoods Associated with Clinical Outcome.

Encouraged by these results, we sought to create a global understanding of cellular interaction between CD8 and tumor cells as well as other cell subsets in the TME. We developed an unsupervised multivariate model to construct spatial meta-clusters based on average distances from CD8 to the centroids of 5 nearest endothelial cells, T_{REG}, CD4 T cells, macrophages, and tumor cells (FIG. 9A). Spatial analysis revealed 11 meta-clusters for CD8 spatial network (FIG. 9B) and 1 ‘unclassified’ CD8 spatial cluster (STAR methods). Each CD8 spatial interaction pattern is distinctive, reflected by different average distances from CD8 to the centroids of each phenotype (FIG. 9C). The distribution of CD8 spatial neighborhoods showed heterogeneity across cases (FIG. 9D). Risk assessment analyses showed that these spatial neighborhoods were associated with response to therapy (FIG. 9E). Neighborhoods were classified as “hazardous” when the odds ratio of refractory/complete response was higher than 1.5 and as “protective” when the odds ratio was less than 0.5. The unclassified CD8 clusters and clusters 1, 2, 4 and 7 (hazardous neighborhoods) had almost 3 times higher odds of being identified in refractory cases compared to clusters 6, 9, 10 and 11 (protective neighborhoods). In the hazardous neighborhoods, CD8 were generally interacting with macrophages, whereas in the protective neighborhoods, CD8 tended to interact more with CD4 T cells (FIG. 9F and FIG. 9G).

To better understand the functional status of CD8 and other immune cells in each of these spatial neighborhoods, we first ordered the clusters from most protective to most hazardous as determined by the increasing Odds Ratio for REF/CR. Our assumption was that immune cells passed through a trajectory of initial activation followed by suppression/exhaustion, and that immune activation would be observed in the protective neighborhoods while suppression/exhaustion would be observed in the hazardous neighborhoods. We first focused on markers that would reveal the functional state of CD8 T cells. When ordered by hazards ratio, Ki-67 and granzyme-B expressions on CD8 tended to covary demonstrating a coherent pattern of initial activation followed by suppression (FIG. 10A). PD-1 seemed to decrease from protective to hazardous neighborhoods, while TIM-3 expression had the opposite pattern (FIG. 10B). These patterns indicate that CD8 functional heterogeneity is at least partially determined by the composition of cells in the immediate neighborhood and these neighborhoods determine clinical responses to chemotherapy.

We then turned our attention to the immune cells within each CD8 neighborhood and examined the expression of inducible markers to gain functional insights into these cells. To determine if a particular marker was up or down regulated in a specific spatial cluster, we constructed a null model to test whether immune phenotype protein expression in the CD8 neighborhood was enriched with respect to proximity to CD8 T cells, or reflective of the general protein expression patterns (FIG. 11A). We compared the observed expression of proteins on the 5 nearest cells to the corresponding average intensity of any 5 randomly selected cells within the same phenotype from the null model. In the “protective” neighborhoods, we observed the presence of Th1-like CD4, characterized by high CXCR3*Tbet joint expression on CD4, M1-like macrophages characterized by low CD206, and less suppressive CCR4^{low} T_{REG} (FIG. 11B, FIG. 11C). The opposite was observed in the “hazardous” neighborhoods where Ki-67 and Granzyme-B expressions were low on CD8, CXCR3*Tbet joint expressions were low on CD4, CD206 was high on macrophages, and CCR4 was high on T_{REG}. Finally, PD-L1/TIM-3/CCR4 joint average expression on tumor cells was higher in the hazardous neighborhoods consistent with our previous observation that this phenotype was associated with poor survival (FIG. 7G, FIG. 11B, FIG. 11C).

Tumor PD-L1/TIM-3/CCR4 Co-Expression Predicts Overall Survival Better than IPI.

To understand how our new biomarkers performed compared to established prognostic markers, we performed Cox proportional hazard regression on the CD8 spatial network summarized at the case level, PD-L1/TIM-3/CCR4 triple positive tumor phenotype, and on classic clinical predictive parameters and compared their survival predictive powers using C-index (Table 3). We found that PD-L1/TIM-3/CCR4 triple positive tumor phenotype was the best predictor of overall survival in DLBCL (C=0.85). We also showed that CD8 spatial network (C=0.75) predicted survival almost as well as the gold standard clinical prognostic predictor IPI (C=0.79) and better than age (C=0.37), EBV status (C=0.44), Hans cell of origin (C=0.57), gender, stage (C=0.71) or LDH level (C=0.72). In joint Cox models, the CD8 spatial feature and triple positive tumor phenotype improved survival predictive power of IPI (C=0.79 to C=0.83).

TABLE 3
Tumor PD-L1/TIM-3/CCR4 co-expression
predicts overall survival better than IPI

| PARAMETERS | C-INDEX |
|---|---|
| Age | 0.37 |
| EBV positive | 0.44 |
| HANS Cell of Origin | 0.57 |
| Gender | 0.61 |
| Stage | 0.71 |
| Elevated LDH | 0.72 |
| CD 8 spatial network | 0.75 |
| IPI | 0.79 |
| IPI*PD-L1/TIM-3/CCR4 + tumor joint model | 0.83 |
| IPI*CD8 spatial feature joint model | 0.83 |
| Tumor PD-L1/TIM-3/CCR4 co-expression | 0.85 |

EBV: Epstein Barr Virus;
LDH: Lactic Acid Dehycrongenase;
IPI: International Prognostic Index

TABLE 4
Resource

| | | |
|---|---|---|
| REAGENT or RESOURCE | SOURCE | IDENTIFIER |

Antibodies

| | | |
|---|---|---|
| BCL-2 (clone EPR17509) | Fluidigm | Cat #3146019D; RRID: AB_2811012 |
| BCL-6 (clone K112-91) | Fluidigm | Cat #3147020D; RRID: AB_2811013 |
| CD134 (polyclonal) | Fluidigm | Cat #3151024D; RRID: AB_2811014 |
| CD183 (clone G025H7) | Biolegend | Cat #353733; RRID: AB_2563724 |
| CD194 (clone 205410) | Fluidigm | Cat #3149003A; RRID: AB_2811015 |
| CD20 (clone H1) | Fluidigm | Cat #3161029D; RRID: AB_2811016 |
| CD206 (clone 5C11) | Fluidigm | Cat #3999999-2 |
| CD3 (Polyclonal) | Fluidigm | Cat #3170019D; RRID: AB_2811048 |
| CD31 (clone C31.3) | Abcam | Cat #ab212709; RRID: AB_2811049 |
| CD34 (clone QBEnd/10) | Abcam | Cat #ab213054; RRID: AB_2811050 |
| CD4 (clone EPR6855) | Fluidigm | Cat #3156033D; RRID: AB_2811051 |
| CD45RA (clone HI100) | Fluidigm | Cat #3155011B; RRID: AB_2810246 |
| CD45RO (clone UCHL1) | Fluidigm | Cat #3173016D; RRID: AB_2811052 |
| CD68 (clone KPI) | Fluidigm | Cat #3159035D; RRID: AB_2810859 |
| CD8α | Fluidigm | Cat #3162034D; RRID: AB_2811053 |
| c-Myc p67 (clone 9E10) | Fluidigm | Cat #3164025D; RRID: AB_2811054 |
| Ephrin B2 | Abcam | Cat #ab233246; RRID: AB_2811055 |
| FoxP3 (clone 236A/E7) | Thermo Fischer | Cat #14-4777-82; RRID: AB_467556 |
| Granzyme B (clone EPR20129-217) | Fluidigm | Cat #3167021D; RRID: AB_2811057 |
| Histone 3 (clone D1H2) | Fluidigm | Cat #3176023D; RRID: AB_2811058 |
| HLA-DR (clone YE2/36 HLK) | Fluidigm | Cat #3174023D; RRID: AB_2811059 |
| ICOS (clone D1K2T) | Fluidigm | Cat #314802ID; RRID: AB_2811060 |
| Ki-67 (clone B56) | Fluidigm | Cat #3168022D; RRID: AB_2811061 |
| LAG-3 (D2G40) | Fluidigm | Cat #3153028D; RRID: AB_2811062 |
| PD-1 (clone NAT105) | Biolegend | Cat #367402; RRID: AB_2565782 |
| PD-L1 (clone 28-8) | Abcam | Cat #ab209889; RRID: AB_2811063 |
| PD-L2 (clone 176611) | Fluidigm | Cat #3172028D; RRID: AB_2811066 |
| pStat3 Y7051 (clone 4/P-Stat3) | Fluidigm | Cat #3158005A; RRID: AB_2661827 |
| Tbet (clone D6N8B) | Fluidigm | Cat #3145015D; RRID: AB_2811067 |
| TIM-3 (clone D5D5R) | Fluidigm | Cat #3154024D; RRID: AB_2811068 |
| Vimentin (clone RV202) | Fluidigm | Cat #3143029D; RRID: AB_2811069 |
| Vista (clone D1L2G) | Fluidigm | Cat #3160025D; RRID: AB_2811070 |

Metal Isotopes

| | | |
|---|---|---|
| 142^{Nd} | Fluidigm | Cat #201142A |
| 143Nd | Fluidigm | Cat #201143A |
| 144Nd | Fluidigm | Cat #201144A |
| 145Nd | Fluidigm | Cat #201145A |
| 146Nd | Fluidigm | Cat #201146A |
| 147Sm | Fluidigm | Cat #201147A |
| 148Nd | Fluidigm | Cat #201148A |
| 149Sm | Fluidigm | Cat #201149A |
| 150Nd | Fluidigm | Cat #201150A |
| 151Eu | Fluidigm | Cat #201151A |
| 152Sm | Fluidigm | Cat #201152A |
| 153Eu | Fluidigm | Cat #201153A |
| 154Sm | Fluidigm | Cat #201154A |
| 155Gd | Fluidigm | Cat #201155A |
| 156Gd | Fluidigm | Cat #201156A |
| 158Gd | Fluidigm | Cat #201158A |
| 159Tb | Fluidigm | Cat #201159A |
| 160Gd | Fluidigm | Cat #201160A |
| 161Gd | Fluidigm | Cat #201161A |
| 162Dy | Fluidigm | Cat #201162A |
| 163Dy | Fluidigm | Cat #201163A |
| 164Dy | Fluidigm | Cat #201164A |
| 166Er | Fluidigm | Cat #201166A |
| 167Er | Fluidigm | Cat #201167A |
| 168Er | Fluidigm | Cat #201168A |
| 169Tm | Fluidigm | Cat #201169A |
| 170Er | Fluidigm | Cat #201170A |
| 172Yb | Fluidigm | Cat #201172A |
| 172Yb | Fluidigm | Cat #201142A |
| 173Yb | Fluidigm | Cat #201173A |
| 174Yb | Fluidigm | Cat #201174A |
| 175Lu | Fluidigm | Cat #201175A |
| 176Yb | Fluidigm | Cat #201176A |

Biological Samples

| | | |
|---|---|---|
| SIGNALSLIDE ® | Cell Signaling | Cat #13747 |
| PD-L1 control | Technology | |

Chemicals, Peptides and Recombinant proteins

| | | |
|---|---|---|
| m-Xylene anhydrous, >99% | Sigma Aldrich | Cat #296325-2L |
| Ethanol | Sigma Aldrich | Cat #459836-2L |
| PBS (Calcium & | Genesee | Cat #25-507 |
| Magnesium free, pH7.4) | Scientific | |
| Trizma base | Sigma Aldrich | Cat #T1503-250G |
| EDTA | Sigma Aldrich | Cat #E1644-250G |
| Tween 20 | Fischer | Cat #BP337-100 |
| | Scientific | |
| BSA (DNase and | Fischer | Cat #BP9706100 |
| Protease-free Powder) | Scientific | |
| Triton X-100 | Sigma Aldrich | Cat #T8787-100ML |
| Cell ID Intercalator (Iridium) | Fluidigm | Cat #201192A |

This retrospective study included a subset of 33 patients from a previously studied cohort of 85 patients diagnosed with de novo DLBCL at Los Angeles County and University of Southern California (USC) Medical Centers between 2002 and 2012. The sub cohort was representative of the primary cohort and was not selected other than looking for samples with adequate remaining tissues for further analyses. This study was approved by the USC Health Sciences Institutional Review Board.

Tissue Microarray and Immunohistochemistry

Two of the six tissue microarray (TMA) blocks from the parent cohort with sufficient remaining tissues representative of viable tumor were obtained from the Pathology archive of Los Angeles County and USC Medical Centers. Immunohistochemistry (IHC) staining was performed on 4-μm tissue sections using DAKO ready-to-use antibodies with the EnVision FLEX and FLEX+ visualization systems (DAKO, Glostrup, Denmark) on an automated immunostainer (Autostainer Link 48, DAKO). Detailed IHC staining protocol and scoring methods have previously been published.

Imaging Mass Cytometry Staining

Three of the six TMAs with optimal quality of remaining tumor tissues from the larger cohort study were selected for this study. The TMAs contained 42 cores of FFPE DLBCL tissues from 33 patients and 2 cores from liver tissues. FFPE sections of 4-μm were baked at 60° C. for 90 minutes on a hot plate, de-waxed for 20 minutes in xylene and rehydrated in a graded series of alcohol (100%, 95%, 80% and 70%) for 5 minutes each. Heat-induced antigen retrieval was conducted on a hot plate at 95° C. in Tris-EDTA buffer at pH 9 for 30 minutes. After blocking with 3% BSA in PBS for 45 minutes, the sections were incubated overnight at 4° C. with a cocktail of 32 antibodies tagged with rare lanthanide isotopes obtained from Fluidigm (Table 2). Titration for PD-1 antibody was performed on tonsil tissue (follicular T helper cells in the germinal center). PD-L1 titration was done on a commercial slide containing formalin-fixed paraffin-embedded cell pallets of HDLM-2 (PD-L1+) and PC-3 (PD-L1-) cell lines from Cell Signaling Technology (Key Resources Table).

Tissue Imaging and Ablation

All cores were evaluated by two pathologists (I.S. and M.H.) to identify region of interest (ROI) on H&E. Slides were analyzed using the Fluidigm Hyperion Tissue Imager system that couples laser ablation with mass spectrometry. Laser beam of 1-μm² spot size was used to ablate tissue area of 1000-μm² per core at a frequency of 200 Hz. The metal isotopes were simultaneously measured and indexed against the location of each spot to generate intensities and digital spatial maps of the ablated tissues. Detailed descriptions of the ablation techniques have been previously described.

Image Analysis Pipeline

The ion counts for each metal-labeled antibody and slide location were compensated for the cross talk between channels then converted to OME-TIFF images. Images for each antibody were scaled using the 95 percentile of the cumulative signal to remove hot spot pixels and normalized across acquisitions.

Image Segmentation

Channels representing distinct morphological features for cell nuclei (i.e. Ir193-DNA Intercalator, Histone H3, foxP3, Ki-67) and membrane staining (i.e. CD8, CD68, CD45RA) were used for the Ilastik pixel classification training to predict nuclei, membrane/cytoplasm and background pixel class using cropped 2× scaled images. The probability maps were segmented using CellProfiler by subtracting the membrane probability map from the nuclei and then expanding the nuclei by 4 pixels.

Quantification and Statistical Analyses

Phenotypic Clustering

The images along with the masks were imported into histoCAT software for initial evaluation. Cell features were extracted and imported into R statistical software environment. Cells with low average intensities across all markers were excluded from the analysis. For each patient, cells were clustered using phenograph clustering algorithm (k=45) based on the intensities for CD3, CD4, CD8, FoxP3, CD68, CD20, CD31, CD45RA and CD45RO. The resulting clusters for all samples were again clustered (k=15) into 14 meta-clusters using the centroids of each cluster that were visualized on tSNE maps and classified as either T_{HELPER}, T_{CYTO}, T_{REG}, macrophages (MAC), tumor cells and endothelial cells.

Expression Assignment for BCL2 and PD-L1

Data driven tuning parameters were computed using linear models that fit BCL2+ and PD-L1+ tumor abundance onto BCL2 and PD-L1 IHC features. For a given cut point, BCL2+ and PD-L1+ tumor abundances were computed and regressed onto the independent BCL2 and PD-L1 IHC features which identified the optimal cut point with the best fit to patient BCL2 and PD-L1 IHC features. The best fit was measured by the linear model with the highest F-statistic.

Expression Assignment for PD-1

PD-1 intensity was tuned so that for a given cut point, the abundance of PD-1+ T cells on the exhausted T cell phenotypes (TIM-3+/LAG-3+CD4, CD8 and T_{REG}) should be higher than on macrophages and tumor cells. For a range of cut points [0.05, 0.55], we compared the abundances of PD-1+TIM-3+CD8, CD4 and T_{REG} to those of PD-1+TIM-3+ macrophages and tumor cells. Additionally, we compared the abundances of PD-1+LAG-3+CD8, CD4 and T_{REG} to those of PD-1+LAG-3+ macrophages and tumor cells. Lastly, the abundances of PD-1+TIM-3+LAG-3+CD8, CD4, and T_{REG} were compared to those of PD-1+TIM-3+LAG-3+ macrophages and tumor cells. For each cut point (range 0.05-0.55), a total relative ratio of PD-1 abundance on each exhausted T cell phenotype was computed, summed and averaged across cases. These ratios were then compared across the different cut points. The optimal cut point was identified as one which gave the highest average PD-1+ relative abundances on exhausted T-cells compared to macrophages and tumor cells.

Expression Assignment for Other Inducible Markers

Intensities of inducible markers on each cell were normalized using inverse hyperbolic sine transformation and by performing min/max normalization scaling across each protein marker feature. Hence, marker intensities were standardized to [0,1] and were considered positive on a cell if the scaled expression level on the cell was >0.5, as described by Keren et al.

Analyses of Complex Phenotypes

Complex Tumor Phenotypes

The inverse hyperbolic sine transformed intensity values were min/max normalized across all markers to scale the data onto [0,1] scale. For each cell, the marker intensity was set to a Boolean cutoff (1: if marker intensity >optimal cut point, 0: otherwise). Annotated tumor phenotypes that were identified as CD20+ were selected for marker combinations in the context of BCL2+/−, CCR4+/−, TIM-3+/−, and PD-L1+/−. We used a Boolean strategy by ordering the phenotype layering using prevalence of positive cells for each marker. The Boolean strategy determined that each cell can only be positive or negative for each marker. The treatment response category for each cell was inherited from its corresponding case's response category (CR versus REF). Chi-square tests were used to compare the association between CD20+BCL2+CCR4+TIM-3+PD-L1+ and CD20+BCL2+CCR4−TIM-3−PD-L1− with respect to treatment response categories. The association was reported if p-value was <0.05.

Complex T-Cell Phenotypes

First, we calculated the abundance of T cell phenotypes using the optimal cut points for CD3, CD4, CD8, CCR4, TIM-3, and FoxP3. Boolean strategy as used to assign combinations of phenotypes in order of their abundance. The total CD8+CCR4+TIM-3+PD-L1+ population was gated on the Boolean set CD3+CD4-CD8+CCR4+TIM-3+FoxP3−PD-L1+ which had N=6095. The total CD8+ cells were gated on FoxP3- and CD4-, (N=24,452). The total CD8+ Boolean sets included CD3+CD4−CD8+CCR4−TIM-3−FoxP3−PD-L1−, CD3+CD4−CD8+CCR4−TIM-3−FoxP3−PD-L1+, CD3+CD4-CD8+CCR4−TIM-3−FoxP3+PD-L1−, CD3+CD4−CD8+CCR4−TIM-3−FoxP3+PD-L1+, CD3+CD4−CD8+CCR4−TIM-3+FoxP3−PD-L1−, CD3+CD4−CD8+CCR4−TIM-3+FoxP3−PD-L1+, CD3+CD4−CD8+CCR4−TIM-3+FoxP3+PD-L1−, CD3+CD4−CD8+CCR4−TIM-3+FoxP3+PD-L1+, CD3+CD4−CD8+CCR4+TIM-3−FoxP3−PD-L1−, CD3+CD4−CD8+CCR4+TIM-3−FoxP3−PD-L1+, CD3+CD4−CD8+CCR4+TIM-3−FoxP3+PD-L1−, CD3+CD4−CD8+CCR4+TIM-3−FoxP3+PD-L1+, CD3+CD4−CD8+CCR4+TIM-3+FoxP3−PD-L1−, CD3+CD4−CD8+CCR4+TIM-3+FoxP3−PD-L1+, CD3+CD4−CD8+CCR4+TIM-3+FoxP3+PD-L1−, CD3+CD4−CD8+CCR4+TIM-3+FoxP3+PD-L1+, CD3+CD4+CD8+CCR4−TIM-3−FoxP3−PD-L1−, CD3+CD4+CD8+CCR4−TIM-3−FoxP3−PD-L1+, CD3+CD4+CD8+CCR4−TIM-3−FoxP3+PD-L1−, CD3+CD4+CD8+CCR4−TIM-3−FoxP3+PD-L1+, CD3+CD4+CD8+CCR4−TIM-3+FoxP3−PD-L1−, CD3+CD4+CD8+CCR4−TIM-3+FoxP3−PD-L1+, CD3+CD4+CD8+CCR4−Tim3+FOXP3+PDL1−, CD3+CD4+CD8+CCR4−TIM-3+FoxP3+PD-L1+, CD3+CD4+CD8+CCR4+TIM-3−FoxP3−PD-L1−″ ″CD3+CD4+CD8+CCR4+TIM-3−FoxP3−PD-L1+, CD3+CD4+CD8+CCR4+TIM-3−FoxP3+PD-L1−, CD3+CD4+CD8+CCR4+TIM-3−FoxP3+PD-L1+, CD3+CD4+CD8+CCR4+TIM-3+FoxP3−PD-L1−, CD3+CD4+CD8+CCR4+TIM-3+FoxP3−PD-L1+, CD3+CD4+CD8+CCR4+TIM-3+FoxP3+PD-L1−, CD3+CD4+CD8+CCR4+TIM-3+FoxP3+PD-L1+(N=28573). The total CD3+CD4+CCR4+TIM-3+PD-L1+ was gated on CD3+CD4+CD8−CCR4+TIM-3+FoxP3−PD-L1+(N=5507). The total CD3+CD4+ was gated on CD3+CD4+CD8−CCR4−TIM-3−FoxP3−PD-L1−, CD3+CD4+CD8−CCR4−TIM-3−FoxP3−PD-L1+, CD3+CD4+CD8−CCR4−TIM-3−FoxP3+PD-L1−, CD3+CD4+CD8−CCR4−TIM-3−FoxP3+PD-L1+, CD3+CD4+CD8−CCR4−TIM-3+FoxP3−PD-L1−, CD3+CD4+CD8−CCR4−TIM-3+FoxP3−PD-L1+, CD3+CD4+CD8−CCR4−TIM-3+FoxP3+PD-L1−, CD3+CD4+CD8−CCR4−TIM-3+FoxP3+PD-L1+, CD3+CD4+CD8−CCR4+TIM-3−FoxP3−PD-L1−, CD3+CD4+CD8−CCR4+TIM3-FoxP3−PD-L1+, CD3+CD4+CD8−CCR4+TIM-3−FoxP3+PD-L1−, CD3+CD4+CD8−CCR4+TIM-3−FoxP3+PD-L1+, CD3+CD4+CD8−CCR4+TIM-3+FoxP3−PD-L1−, CD3+CD4+CD8−CCR4+TIM-3+FoxP3−PD-L1+, CD3+CD4+CD8−CCR4+TIM-3+FoxP3+PD-L1−, CD3+CD4+CD8−CCR4+TIM-3+FoxP3+PD-L1+, CD3+CD4+CD8+CCR4−TIM-3−FoxP3−PD-L1−, CD3+CD4+CD8+CCR4−TIM-3−FoxP3−PD-L1+, CD3+CD4+CD8+CCR4−TIM-3−FoxP3+PD-L1−, CD3+CD4+CD8+CCR4−TIM-3−FoxP3+PD-L1+, CD3+CD4+CD8+CCR4−TIM-3+FoxP3−PD-L1−, CD3+CD4+CD8+CCR4−TIM-3+FoxP3−PD-L1+, CD3+CD4+CD8+CCR4−TIM-3+FoxP3+PD-L1−, CD3+CD4+CD8+CCR4−TIM-3+FoxP3+PD-L1+, CD3+CD4+CD8+CCR4+TIM-3−FoxP3−PD-L1−, CD3+CD4+CD8+CCR4+TIM-3−FoxP3−PD-L1+, CD3+CD4+CD8+CCR4+TIM-3−FoxP3+PD-L1−, CD3+CD4+CD8+CCR4+TIM-3−FoxP3+PD-L1+, CD3+CD4+CD8+CCR4+TIM-3+FoxP3−PD-L1−, CD3+CD4+CD8+CCR4+TIM-3+FoxP3−PD-L1+, CD3+CD4+CD8+CCR4+TIM-3+FoxP3+PD-L1−, CD3+CD4+CD8+CCR4+TIM-3+FoxP3+PD-L1+(N=33,227).

Development of M-Score

To summarize protein expression at the case level we defined an M-score, a non-parametric measure which did not involve identifying a threshold value to determine cell positivity. The normalized marker intensity across all cells was cut into 10 blocks based on the quantiles of the total dynamic range. Each block was assigned the average intensity of the cells that held membership to the corresponding block. For each case, and for each block, the number of cells were counted. The M-score was measured for each case, as a linear combination of the number of cells in a given block and the average intensity of the block. The M-score per patient was compared to the case positive proportions and Pearson's correlation test was used, as well as linear regression to evaluate their relationship.

$${M}_{j} = {\sum }_{i = 1}^{10} {μ}_{ij} {c}_{ij}$$

Where Mⱼ denotes the M-score of selected protein intensity for the j^{th} patient, μᵢ denotes the average protein intensity of the i^{th} quantile block, and ci denotes the number of cells in the i^{th} quantile block

Survival Analyses

Exploratory Univariate Survival Model

Univariate survival models were computed for case abundance proportions of positive cell counts relative to the total cells per case. The cell positivity of a given protein was determined by the protein intensity which exceeded the threshold. For univariate exploratory analyses, we did not examine case positive abundances in a given phenotypic context, but general relative abundances across all cells. The proteins tested were BCL2, BCL6, CD20, CD206, CD3, CD4, CD30, CD31, CD4, CD45RA, CD45RO, CD68, CD8, EphrinB2, FoxP3, HLA-DR, C-MYC, CCR4, CD134, CXCR3, Granzyme-B, ICOS, Ki-67, LAG-3, PD-1, TIM-3, Vimentin, Vista, and pSTAT. Survival follow up data was obtained as overall survival (days), and death during the observation window was considered as ‘events’, otherwise the event was censored. There were 33 subjects, with 9 events, the mean survival time was 1159 days. The Harrell's concordance index (C-index) was calculated using 3-fold cross validation. We used a variant of the Cox proportional hazards model to estimate the survival models that included a Gaussian prior on the coefficients, and the variance prior was estimated by empirical Bayes. These shared priors included a ridge penalty to help provide improved model coefficient estimations. For a given univariate feature previously described, a Cox proportional hazards risk estimate was compounded as a linear combination with the corresponding patient case relative abundances to form compounded risk measures, which were then cut into 2 risk groups (high/low) by a median-based cut. The risk groups were then used for a Kaplan-Meier patient survival model. Significant differences in survival times of the 2 risk groups was assessed using the Likelihood-Ratio-Test (LRT) coupled with p-value alpha level of 0.05.

Survival Analysis Using Cut Points

For a given phenotype, the markers' normalized and scaled intensity values that exceeded a defined cut point (default 0.5, PD-1: 0.35, PD-L1: 0.34) were counted relative to the total number of cells belonging to a phenotype context per case. If a phenotype context had no cells represented in a case, the abundance was modeled as 0. For complex phenotype combinations, the cells were counted positive if all the phenotypic protein intensities exceeded the default/tuned thresholds.

Survival Analysis Using M-Score.

The derivation of M-score for co-expression of PD-L1, TIM-3 and CCR4 required that the proteins on the cells were co-expressed. The normalized protein intensity values were on [0,1] scale, and co-expression was defined as the multiplicative product of the protein markers which preserved the [0,1] scale. For complex phenotypes, the M-score was computed on the co-expression intensity product of PD-L1, TIM-3 and CCR4 on CD4, CD8 and tumor cells.

Spatial Analysis

Spatial Networking

Spatial neighborhood analyses were performed in the context of CD8 interaction (or the interaction of the index cell) with its first 5 nearest CD4, T_{REG}, macrophages, endothelial and tumor cells. The centroid location of the first 5 nearest neighbors to CD8 was determined by averaging the X,Y coordinates of the 5 nearest cells belonging to each phenotype previously described. For each CD8 cell (or index cell), the distances to the centroids of its 5 nearest CD4, T_{REG}, macrophages, endothelial and tumor cells were standardized by dividing the observed centroid distance by the total number of cells of the corresponding phenotype per case. For all cases, the standardized distances were then scaled by the cohort average abundance of the corresponding phenotype. For the j^{th} CD8 cell (or index cell) and i^{th} phenotype (pi), the centroids were standardized using the following formula:

$${( CD 8 dist . to {p}_{i} centroid )}_{j} = ( cohort avg . abundance {p}_{i} ) \times \frac{{( CD 8 dist . to {p}_{i} centroid )}_{j}}{{( total {p}_{i} abundance )}_{j}}$$

The standardized distances were then mean centered and scaled by their standard deviation to derive Z-scores. The Z-scores of the distances were clustered using Phenograph (k=45) for each case independently, and then meta-clustered (k=10) using the centroid distances per individual case phenograph clusters. The meta-clusters were plotted using ‘UMAP’

Filtering Out Distant Interactions

The spatial network model required CD8 distances to all centroids to be less than 200 μm, which identified 41,205 CD8 cells. This criterion filtered 23.44% of CD8 cells from the clustering spatial network. Before filtering, the 75^{th} quartile of CD8 distances to endothelial, CD4, tumor, T_{REG}, and macrophage centroids was 59.55 μm, 21.19 μm, 9.53 μm, 33.51 μm and 23.42 μm, respectively.

Protein Quantification for Spatial Clusters

To infer the functional statuses of CD8 T cells and the 5 nearest neighbors, we averaged the normalized signal intensity values [0,1] of selected proteins of interest on the following phenotypes: Ki-67 and Granzyme-B on CD8; TIM-3, PD-1, CCR4, CXCR3, and Tbet on CD4; FoxP3, TIM-3, PD-L1, and CCR4 on T_{REG}; CD206 and PD-L1 on macrophages; and PD-L1, TIM-3, and CCR4 on tumor cells.

In order to understand the stochasticity of protein expression in the observed CD8 neighborhoods, we constructed a null model to represent the general population. The null model was constructed for each phenotype by random sampling any 5 phenotypic cells, then computing the average protein intensity. This process was repeated 39 times to generate the null distribution. Z-scores of cluster deviation from the null were computed by comparing the observed average cluster neighborhood protein intensity to the average cluster null intensity, then divided by the average null standard deviation.

CD8 Spatial Network Survival Model

The CD8 spatial network features used in the survival model were computed by the spatial cluster size (FIG. 9D) relative to the total cells per case, and then scaled by the number of CD8 cells per corresponding case. Due to limited sample size, we constructed a summary model by averaging the spatial clusters abundances based on the sign of the Cox proportional hazards multivariate model. The spatial clusters case relative abundance with Cox hazards estimates >0 were averaged. Similarly, spatial clusters abundances with Cox hazards estimates <0 were averaged. Hence, we reduced the spatial features used in the Cox survival model from 12 features to 2. Using the 2 summary features, we re-performed the Cox proportional hazards summary model to compute the C-index score.

### Example 3

Cancer immunotherapy, and checkpoint inhibition in particular, is poised to radically transform our approach to treating cancer. Recent clinical successes with checkpoint inhibitors and immuno-oncology agents have demonstrated the importance of the immune system in controlling and treating cancers. Early indications that tumor cells can generate an antitumor immune response were evidenced by the varying degrees of immune cell infiltration found in the tumor immune microenvironment (TME). Despite the presence of this immune infiltrate, is has long been suspected that immune function is defective or inhibited by the TME. Lymphomas are divided into Hodgkin and non-Hodgkin lymphoma, with diffuse large B cell lymphoma (DLBCL) being the most subtype of non-Hodgkin lymphoma. Both types of lymphoma demonstrate expression of PDL-1 on tumor cells but they have very different responses to treatment with PD1/PD-L1 targeting agents. For example, two recent clinical trials with nivolumab demonstrated response rates in Hodgkin lymphoma of 87%, while responses in DLBCL were less than 10%.

While not wishing to be bound by any particular theory, we believe that a better characterization of spatial architecture of the tumour microenvironment (TME) in lymphoma will help explain differences in responses to PD1/PDL-1 inhibitors and guide future targeted immunotherapies for these patients. Similar studies in this area have been limited by technical challenges. Single cell techniques such as CyTOF or single cell RNA-seq, rely on tissue disruptions and all spatial information is lost. Current multiplex tissue techniques such as multiplex IHC/IF are limited to 6-8 markers, while spatial transcriptomics lack single cell resolution. In the present study, we characterized TME components, including immunopheno-types, frequency and spatial interaction, in DLBCL using imaging mass cytometry (IMC), which allows high-dimensional, single-cell and spatial analysis of FFPE tissues at sub-cellular resolution. Using a panel of 32 antibodies, IMC was performed 41 tissue microarray cores from 33 DLBCL cases. Using both supervised gating and unsupervised clustering approaches, IMC data were analyzed for relevant immunophenotypes and compared across clinical outcome groups. The TME was primarily composed of CD4+T-helper cells (13.1%±1.9%), CD8+ cytotoxic T cells (10.8%±1.1%), CD68+ macrophages (6.3%±0.9%), FoxP3+ regulatory T cells (2.7%±0.5%), while the bulk of samples were tumor cells 58.1%±3.4%. We identify a population of CD8 T cell phenotype co-expressing PD-L1/TIM-3/CCR4 to be enriched in refractory DLBCL. More importantly, we demonstrated a negative association between OS and CCR4/TIM-3− expressing T_{REG}, T_{HELPER} and tumour cells, indicating that DLBCL patients could potentially benefit from TIM-3 and CCR4 inhibitors presently under clinical investigation. To describe the spatial architecture of the tumor microenvironment, we developed a novel clustering approach to group CD8 cells by their distance to tumor and other components of the microenvironment. Using this approach we identified 3 dominant CD8 clusters characterized by their distance to tumor, T_{REG} or macrophages. We were able to demonstrate phenotypic differences in activation (ki-67, granzyme B) or exhaustion (PD-1, TIM3, LAG3) in these different spatial clusters and associate these with response to chemotherapy. Finally, we show that sub-setting our analysis of CD8 phenotypes based on their spatial location to other cells improved our ability to predict overall survival in the cohort.

Together, these results show that deep profiling of the immune architecture of the tumor microenvironment is associated with clinical outcomes in DLBCL, and that spatial analysis of immune cells should be explored as a potential biomarker for patients treated with immunotherapies.

### Example 4

Background Diffuse large b cell lymphoma (DLBCL) being the most subtype of non-Hodgkin lymphoma. Despite evidence of expression of PDL-1 on lymphoma cells, less than 10% of DLBCL patients respond to PD1 therapy. We hypothesize that a better characterization of spatial architecture of the tumour microenvironment (TME) in lymphoma will help explain differences in responses to PD1/PDL-1 inhibitors and guide future targeted immunotherapies for these patients.

Methods. Here we characterized the TME in DLBCL using imaging mass cytometry (IMC), which allows high-dimensional, single-cell and spatial analysis of FFPE tissues at sub-cellular resolution. Using a panel of 32 antibodies, IMC was performed 41 tissue microarray cores from 33 DLBCL cases. IMC images were analyzed for relevant immunophenotypes, the spatial architecture of those phenotypes and compared to clinical outcomes to identify immune contexture based biomarkers.

Results. Phenograph was used to cluster tumor and immune cells based on phenotype (FIG. 1A). Immune cell represented 33% of the cells represented by CD4 (36%), CD8 (30%), macrophages (26%) and T_{REG} (8%) (FIG. 1B). Immune cell infiltration in individual tumor samples ranged from 7% to 75% with marked heterogeneity. (FIG. 1D-FIG. 1E). Analysis of immune marker expression on tumor cells identified co-expression of PD-L1/CCR4/TIM3 to be highly prognostic for overall survival (p=0.003, FIG. 1G).

To characterize the patterns of spatial interaction in the TME, we developed an unsupervised multivariate model to construct spatial meta-clusters based on average distances from CD8 to the centroids of 5 nearest endothelial cells, T_{REG}, CD4 T cells, macrophages, and tumor cells (FIG. 9A). Spatial analysis revealed 11 meta-clusters for CD8 T cell interactions (FIG. 9B). Meta-clusters 2, 6, 8 and 11 were the 4 most dominant patterns of CD8 spatial interaction in the TME. Each CD8 spatial interaction pattern is distinctive with case to case heterogeneity (FIG. 9C-FIG. 9D). Risk assessment analyses of spatial clusters 1, 2 and 4 (“hazardous”) had almost 3 times higher odds of being identified in refractory cases compared to clusters 3, 5 and 6 (“protective”) (FIG. 9E). In the “protective” spatial neighborhoods, we observed the presence of activated CD8, Th1-like CD4, and less suppressive T_{REG} phenotypes, with opposite in “hazardous” areas (FIG. 12A-FIG. 12B). TIM-3 expression was high both on T cells and tumor cells in the “hazardous” neighborhoods.

Our new approach to spatial analysis of the immune architecture reveals clinically relevant insights into the TME.

Many variations and alternative elements have been disclosed in embodiments of the present invention. Still further variations and alternate elements will be apparent to one of skill in the art. Various embodiments of the invention can specifically include or exclude any of these variations or elements.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Various embodiments of this application are described herein, including the best mode known to the inventors for carrying out the application. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A method of performing complex spatial analysis of a tissue sample, comprising:
identifying one or more phenotypic clusters of cells relative to an index cell in a tissue sample or an image of the tissue sample, wherein each phenotypic cluster is identified based on two or more neighboring cells of a same phenotype; and
measuring a marker intensity of each phenotypic cluster of cells.

2. The method of claim 1, wherein the tissue sample is from a subject diagnosed with a cancer, suspected of having a cancer, or having a refractory or relapsed cancer; and the method identifies one or more phenotypic clusters of cancer cells and one or more types of immune cells, macrophages and endothelial cells in the tissue sample.

3. The method of claim 1, wherein the tissue sample comprises a tumor tissue selected from the group consisting of diffuse large B cell lymphoma (DLBCL), Hodgkin's lymphoma, other non-Hodgkin lymphoma, breast cancer, ovarian cancer, prostate cancer, melanoma, and a combination thereof.

4. The method of claim 1, wherein the index cell is a tumor cell, and the marker comprises one or more immune cell markers or macrophage markers; or wherein the index cell is an immune cell, and the marker comprises one or more tumor cell markers.

5. (canceled)

6. (canceled)

7. The method of claim 1, further comprising calculating a centroid location of each phenotypic cluster of cells by averaging the X,Y coordinates of the two or more neighboring cells of the same phenotype, and calculating a centroid distance between the centroid location of each phenotypic cluster to the index cell; optionally further comprising standardizing the distances between the index cell and each centroid location by dividing each centroid distance by the total number of cells of the corresponding phenotype.

8. The method of claim 7, wherein the index cell is an immune cell and each cluster is a tumor phenotype, and the method further comprising:
identifying a first, tumor-core-immune-desert zone which comprises a phenotypic cluster whose centroid distance to the index cell is no less than a first threshold or the farthest in all clusters, or within which immune activity is lowest in all clusters as characterized by substantially absent of proliferative CD8+ T cells, macrophages or T_{REG} cells;
identifying a second, tumor-dispersed-immune-activation zone which comprises a phenotypic cluster whose centroid distance to the index cell is no greater than a second threshold or the shortest in all clusters, or within which immune activity is activated as characterized by substantial presence of proliferative CD8+ T cells, macrophages and T_{REG} cells; and/or
identifying a third, tumor-immune-interface zone which comprises a phenotypic cluster whose centroid distance to the index cell is between the first threshold and the second threshold or between the first zone and the second zone, or within which immune activity is suppressed as characterized by substantial presence of exhausted CD8+ T cells and suppressive T_{REG} cells.

9. The method of claim 8, wherein the first threshold is 40 μm, 41 μm, 42 μm, 43 μm, 44 μm, 45 μm, 46 μm or 47 μm, and the second threshold is 12 μm, 13 μm, 14 μm, 15 μm, 16 μm, 17 μm, 18 μm, 19 μm, or 20 μm.

10. (canceled)

11. The method of claim 8, further comprising performing a mutation screen of a plurality of genes on the tissue sample, wherein gene mutations of cells within at least one of the zones are identified; and/or further comprising performing cell-of-origin analysis on the tissue sample by profiling gene expressions in the cells, wherein B cells within at least one of the zones are identified as one of germinal center B-cell (GCB), activated B-cell (ABC), double-expressor, and double-hit or triple-hit lymphoma.

12. The method of claim 11, wherein the plurality of genes comprises BCL2, BCL6, EZH2, CD79, MYD88^{L265P}, TP53, SGK1, 9p24 Gain, NOTCH1, NOTCH2, or a combination of any two, three, four, five, six, seven, eight, nine or ten genes thereof.

13. (canceled)

14. The method of claim 1, wherein the two or more neighboring cells comprise 5-10, 11-15, 16-20, 21-25, or 26-30 neighboring cells, each being adjacent to at least one other cell in the cluster, and the method identifies 2-4, 5-10, 11-15, 16-20, 21-25, 26-30, 31-35, 36-40, 41-45, or 46-50 different phenotypic clusters of cells; and wherein the two or more neighboring cells of the same phenotype are the closest in distance of that phenotype to the index cell.

15. (canceled)

16. (canceled)

17. The method of claim 1, further comprising first performing an imaging mass cytometry (IMC) to generate an image of the tissue sample, or wherein the tissue sample is first subjected to mass spectrometry imaging to generate an image of the tissue sample, wherein measuring a marker intensity includes measuring a plurality of markers comprising any one, partial combination, or all 32 antigens or markers according to Table 2, and optionally the cells of the tissue sample comprise a label selected from the group consisting of an antibody label, an isotope label, a fluorescent label, a fluorochrome label, a fluorophore label, and combinations thereof.

18. (canceled)

19. (canceled)

20. A method for profiling a tumor microenvironment, comprising:
performing imaging mass cytometry (IMC) on a tumor sample from a subject in need thereof, thereby obtaining imaging mass cytometry data of the tumor sample; and
performing a method of complex spatial analysis according to claim 8 on the tumor sample.

21. The method of claim 20, further comprising providing a prognosis for survival of the subject, and/or selecting or administering a treatment for the subject, based on findings in the complex spatial analysis; wherein the subject has a cancer selected from the group consisting of diffuse large B cell lymphoma (DLBCL), Hodgkin's lymphoma, breast cancer, ovarian cancer, prostate cancer, melanoma, and combinations thereof.

22. (canceled)

23. (canceled)

24. A method of identifying and treating a subject with diffuse large B cell lymphoma (DLBCL), comprising:
detecting an increased amount of TIM-3+ T cells in a lymphoma sample of the subject relative to a control, said control comprising a subject with Hodgkin's lymphoma or with no lymphoma,
administering to the subject an effective amount of an anti-TIM-3 antibody.

25. (canceled)

26. The method of claim 24, further comprising administering to the subject an effective amount of engineered immune cells which expresses chemokine receptor CXCR3; and wherein the method does not include administering a PD-1 antagonist to the subject.

27. (canceled)

28. A method of predicting response to a therapy in a subject having a cancer, comprising performing complex spatial analysis on a tissue sample of the subject according to claim 8, wherein the marker intensity of at least one of the phenotypic cluster is indicative of response to the therapy; and
wherein if the therapy comprises a chemotherapy, then the subject's response to the chemotherapy is indicated as ineffective if:
the cancer cells express a combination of PD-L1, TIM-3 and CCR4,
suppressive TREG or PD-L1+M2 macrophages are present in the cancer cells' microenvironment,
a largest tumor cell cluster expresses PD-L1, or
a largest tumor cell cluster expresses Ki67 and CCR;
or
wherein if the therapy comprises a drug that targets PD-1, then the subject's response to the drug that targets PD-1 is indicated as ineffective if there is an increased presence of TIM-3−PD-L1+ macrophages and/or TIM-3+ T cells within microenvironment of malignant B-cells.

29. The method of claim 28, wherein the complex spatial analysis is performed from a tissue sample collected from the subject after initiation of the therapy.

30. (canceled)

31. (canceled)

32. A method for calculating an intensity-weighted abundance score (M-score) of a marker in a tissue sample, comprising:
measuring a normalized marker intensity across all cells in the tissue sample or an image of the tissue sample;
dividing the tissue sample or the image of the tissue sample into a plurality of blocks based on quantiles of a total dynamic range of the tissue sample or the image of the tissue sample;
assigning an average intensity of cells in each block of the plurality of blocks; and
counting a number of cells in each block of the plurality of blocks;
wherein the intensity-weighted abundance score of the marker is obtained by a linear combination of the number of cells in each block and the average intensity of the respective block.

33. The method of claim 32, further comprising first using imaging mass cytometry (IMC) to generate an image of the tissue sample, or wherein the tissue sample is first subjected to mass spectrometry imaging to generate an image of the tissue sample; wherein optionally the cells are labeled with a label selected from the group consisting of an antibody label, an isotope label, a fluorescent label, a fluorochrome label, a fluorophore label, and combinations thereof.

34. The method of claim 32, wherein the plurality of blocks is 2-5, 6-10, 10-15, or 16-20 blocks, and wherein the intensity-weighted abundance score is calculated for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36-40, 41-45, 46-50, 51-55, 56-60, 61-65, 66-70, or 71-75 markers in the tissue sample.

35. (canceled)

36. (canceled)
